SEQUENCE LISTING (17 sequences)

SEQ ID NO: 1 - protein, 682 aa, Bos taurus
MPRAPRCRAVRALLRASYRQVLPLAAFVRRLRPQGHRLVRRGDPAAFRALVAQCLVCVPWDAQPPPAAPSFRQVSCLKELVARVVQRLCERGARNVLAFGFTLLAGARGGPPVAFTTSVRSYLPNTVTDTLRGSGAWGLLLHRVGDDVLTHLLSRCALYLLVPPTCAYQVCGPPLYDLRAAAAAARRPTRQVGGTRAGFGLPRPASSNGGHGEAEGLLEARAQGARRRRSSARGRLPPAKRPRRGLEPGRDLEGQVARSPPRVVTPTRDAAEAKSRKGDVPGPCRLFPGGERGVGSASWRLSPSEGEPGAGACAETKRFLYCSGGGEQLRRSFLLCSLPPSLAGARTLVETIFLDSKPGPPGAPRRPRRLPARYWQMRPLFRKLLGNHARSPYGALLRAHCPLPASAPRAGPDHQKCPGVGGCPSERPAAAPEGEANSGRLVQLLRQHSSPWQVYGLLRACLRRLVPAGLWGSRHNERRFLRNVKKLLSLGKHGRLSQQELTWKMKVQDCAWLRASPGARCVPAAEHRQREAVLGRFLHWLMGAYVVELLRSFFYVTETTFQKNRLFFFRKRIWSQLQRLGVRQHLDRVRLRELSEAEVRQHQEARPALLTSRLRFVPKPGGLRPIVNVGCVEGAPAPPRDKKVQHLSSRVKTLFAVLNYERARRPGLLGASVLGMDDIHRA

SEQ ID NO: 2 - DNA, 2048 bp, synthetic construct
  source 1..2048: mol_type = other DNA
atgccgcgcgcgcccaggtgccgggccgtgcgcgcccttctgcgggccagctaccggcaggtgctgcccctggccgccttcgtacggcgcctgcggccccagggccaccggcttgtgcggcgcggggacccggcggccttccgcgcgctggtggctcagtgcttggtgtgcgtgccctgggacgcgcagccgccccctgccgccccgtccttccgccaggtgtcctgcctgaaggagctggtggccagagtcgtgcagaggctctgcgagcgcggcgcgaggaacgtgctggccttcggcttcacgctgctggccggggcccgcggcgggccgcccgtggccttcacgaccagcgtacgcagctacctgcccaacacggtaaccgacacgctgcgcggcagcggcgcctgggggctgctgctgcaccgcgtgggcgacgacgtgctcacccacctgctgtcgcgctgcgcgctctacctgctggtgcccccgacctgcgcctaccaggtgtgtgggccgccgctctatgacctccgcgccgccgccgccgccgctcgtcggcccacgcggcaagtgggcgggacccgggcgggcttcggactcccgcgcccggcctcgtcgaacggcggccacggggaggccgaaggactcctggaggcgcgggcccagggcgcgaggcggcgtcgcagtagcgcgcggggacgactgcctccagccaagaggcccaggcgcggcctggagcccgggcgggatctcgaagggcaggtggcccgcagcccgccccgcgtggtgacacctacccgagacgctgcggaagccaagtctcggaagggcgacgtgcccgggccctgccgcctcttcccgggcggcgagcggggtgtcggctccgcgtcctggcggctgtcaccctcggagggcgagccgggtgccggagcttgcgctgagaccaagaggttcctttactgctccggcggtggcgaacagctgcgccgctccttcctgctctgctccctgcctcccagcctggccggggcgcggacactcgtggaaaccatctttctggactcgaagcccgggccgccaggggctccccgccggccgcgccgcctgcccgcgcgctactggcagatgcggcccctgttccggaaactgcttgggaaccacgcgcggagcccctatggcgcgctgctcagggcgcactgcccgctgccggcctctgcgccccgggcggggccagaccatcagaagtgccctggtgttgggggctgcccctctgagaggccggccgctgcccccgagggcgaggcgaactcagggcgcctggtccagctgctccgccagcacagcagcccctggcaggtgtacgggctcctgcgggcctgtcttcgccgcctggtgcccgccggcctctggggctcccggcacaacgagcggcgcttcctgcggaacgtgaagaagctcctctccctggggaagcacggcaggctctcgcagcaggagctcacgtggaagatgaaggtgcaggactgcgcctggctgcgcgcgagcccaggggctcgctgcgtgcccgccgcggagcaccgccagcgcgaggccgtcctgggtcgcttcctgcactggctgatgggcgcctacgtggtggagctgctcaggagcttcttctacgtcacagagaccacgttccagaagaaccggctcttcttcttccggaagcgcatctggagccagctgcagcgcctgggcgtcagacaacacttagaccgtgtgcggcttcgagaactgtcagaagcagaggtcaggcagcaccaggaggccaggccggctctgctgacatccaggctccgtttcgtccccaagcccggcgggctgcggcccatcgtgaacgtgggctgtgttgagggcgccccggcaccgcccagagacaagaaggtgcagcatctcagctcacgggtcaagacgctgttcgcggtgctgaactacgagcgagctcggcggcctggcctcctgggggcctcggtgctgggcatggacgacatccacagggcctg

SEQ ID NO: 3 - protein, 326 aa, Bos taurus
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKINKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNTGELESDSGSDKANSPAGGIPSTSSCLPSPSTPVQSPHPQFPHISSTMNGTSSSPSGNHQSSFANRPRKSSVNGSSATSSG

SEQ ID NO: 4 - DNA, 981 bp, synthetic construct
  source 1..981: mol_type = other DNA
atgcacaggactacacgcattaaaatcactgagctcaatccccacttgatgtgcgtcctctgcggaggttacttcatcgacgccactacaatcattgagtgtcttcatagtttttgcaagacttgcattgtgagataccttgaaacgtcaaagtattgtcctatttgtgatgtccaggtgcacaagacccgacctttgcttaacatcaggtcagataaaacccttcaagacatagtctataagctggtgccgggcctcttcaagaacgaaatgaagaggcggcgggacttctatgcagcccacccaagtgctgatgctgcgaatgggtcaaatgaagataggggtgaagtggctgatgaagacaaaagaatcatcacggacgatgagataattagtctttccattgaattcttcgatcaaaatagattggaccgcaaaatcaacaaggacaaagaaaaaagtaaagaggaggtcaacgacaagcggtatttgcgctgtccggcagcgatgacagtgatgcacctcaggaagttccttagatctaagatggacataccgaacacctttcaaattgatgttatgtatgaagaggaacccctcaaagattactacacgcttatggatattgcctacatatatacatggcgaaggaacggtccacttccactgaaatatagggtgcgacctacgtgcaagcgcatgaagataagccaccagcgggatggacttacaaatacgggtgaactggaaagcgatagcgggtctgataaagccaatagcccagctggcggaataccatctacatccagttgtctgcccagtccctccacgccagtgcaaagtccacatccacaatttcctcatataagctccacgatgaacggaacctctagcagtccctctggaaaccatcagagttctttcgctaaccgaccgcggaagtcttcagttaatggtagctctgcaacaagctccggctga

SEQ ID NO: 5 - protein, 303 aa, Bos taurus
MATSRYEPVAEIGVGAYGTVYKACDPHSGHFVALKSVRVPNGGGAGGGLPISTVREVALLRRLEAFEHPNVVRLMDVCATARTDRETKVTLVFEHVDQDLRTYLDKAPPPGLPVETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFSPRGPRPVQSVVPELEESGAQLLLEMLTFNPHKRISAFRALQHSYLHKAEGDAE

SEQ ID NO: 6 - DNA, 912 bp, synthetic construct
  source 1..912: mol_type = other DNA
atggccacaagccgatacgaaccagtggcagagataggtgtcggggcttacggaactgtttataaggcttgtgatccgcattccggacattttgtcgctctgaaatccgtgcgcgtgccaaatgggggaggagcaggtggaggactcccaatttctactgttagagaggttgcattgctgagacgcctggaggcctttgagcatcccaacgtggttaggcttatggatgtttgtgccacggcccggaccgatcgggagacgaaagtcacactcgtctttgagcacgtggaccaggatttgaggacctatctggacaaagccccaccacccgggctgccagttgagactatcaaggaccttatgagacagtttcttcgaggcctcgatttcctccacgctaactgcatcgtccatagagacctgaagcccgagaatattttggttacctctggcggcaccgttaaattggcggatttcgggttggcacgaatttattcatatcagatggccctgaccccagtcgtcgttacgctttggtaccgcgcccctgaggttctcctccaaagtacttacgctactcctgttgacatgtggtcagttggatgtatttttgctgaaatgtttcggagaaaaccgctgttttgcgggaactctgaagccgatcagttgggaaaaatatttgacttgatcggactcccgccggaagatgactggccccgggatgtttctcttccgcgcggggcattctctcccagaggtccacggccagttcagagcgtcgtgcctgaacttgaggaatcaggggctcagcttctgctcgagatgcttacttttaatccacacaaacggatcagcgcgttccgcgcactgcaacactcctatcttcataaggcagaaggggacgcggagtga

SEQ ID NO: 7 - protein, 295 aa, Bos taurus
MAHQLLCCEMETIRRAYPDANLLNDRVLRAMLKAEETCAPSVSYFKCVQKEILPSMRKIVATWMLEVCEEQKCEEEVFPLAMNYLDRFLSLEPVKKSRLQLLGATCMFVASKMKETIPLTAEKLCIYTDNSIRPDELLHMELVLVNKLKWNLAAMTPHDFIEHFLSKMPVAEENKQIIRKHAQTFVALCATDVKFISNPPSMVAAGSVAAAAQGLHLGSANGFLSYHRLTRFLSKVIRCDPDCLRACQEQIEALLESSLRQAQQQNLDPKAAEEEEEEEEVDLACTPTDVRDVNI

SEQ ID NO: 8 - DNA, 888 bp, synthetic construct
  source 1..888: mol_type = other DNA
atggcacatcagctgctgtgttgtgaaatggagaccattcggcgcgcctatcccgatgctaatcttcttaatgatcgagtgctgcgggccatgctgaaggctgaagaaacgtgcgctccgtcagtctcttatttcaaatgcgtgcaaaaggagattctccctagtatgcgaaaaattgtggccacatggatgctggaggtgtgtgaagagcagaagtgtgaagaagaggtcttcccacttgcaatgaactatcttgatagatttcttagtctggagccggtgaaaaagtcacggctccagttgctcggagctacttgcatgtttgttgcttccaaaatgaaagaaacaatcccactcacagcagagaagttgtgcatctacactgataactctattcgacctgacgagctgcttcacatggagctggttctcgtcaataagctgaaatggaacctcgcagcgatgacaccccatgacttcatagagcattttctttcaaaaatgcctgtcgccgaggagaataagcagattatacgcaaacacgcccaaacatttgtggcgctttgtgcgaccgacgtcaaatttatctctaacccaccatcaatggttgccgctggcagcgttgctgccgcggctcaaggactgcatctgggctcagcaaacggatttttgtcctatcatcgcctgacaagattcctgtctaaagtcatcagatgcgatcccgactgcttgcgagcatgtcaggaacaaatagaggccctgttggagtcaagccttagacaggcgcaacagcagaatctcgacccaaaagcagctgaggaggaggaagaggaagaagaagttgacttggcttgtacacccacggatgtcagggacgttaacatttga

SEQ ID NO: 9 - protein, 681 aa, Bos taurus
MDEGYFCAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTENEASPSSMPDGTPPPQEAEEPSLLKKLLLAPANTQLSYNECSGLSTQNHANHNHRIRTNPAVVKTENSWSNKAKSICQQQKPQRRPCSELLKYLTTNDDPPHTKPTENRNSSRDKCTSKKKAHTQSQTQHLQAKPTTLSLPLTPESPNDPKGSPFENKTIERTLSVELSGTAGLTPPTTPPHKANQDNPFRASPKLKPSCKTVVPPPSKKARYSESSCTQGSNSTKKGPEQSELYAQLSKTSVLTSGHEERKAKRPSLRLFGDHDYCQSINSKTEILVSTSQELHDSRQLENKDAPSSNGPGQIHSSTDSDPCYLRETAEVSRQVSPGSTRKQLQDQEIRAELNKHFGHPSQAVFDDKADKTSELRDSDFSNEQFSKLPMFINSGLAMDGLFDDSEDESDKLNSPWDGTQSYSLFDVSPSCSSFNSPCRDSVSPPKSLFSQRPQRMRSRSRSFSRHRSCSRSPYSRSRSRSPGSRSSSRSCYYYESGHCRHRTHRNSPLCASRSRSPHSRRPRYDSYEEYQHERLKREEYRREYEKRESERAKQRERQRQKAINQQVSSHFPLQEERRVIYVG

SEQ ID NO: 10 - DNA, 2043 bp, synthetic construct
  source 1..2043: mol_type = other DNA
atggatgaggggtacttttgtgctgcactcgtcggcgaggaccaacccctttgcccggacctccccgagttggacctcagtgagcttgacgtcaacgatttggacacggatagctttctgggtgggcttaaatggtgttctgaccaatccgagatcatatccaatcagtataataacgaaccgagtaacattttcgagaaaatagatgaggagaatgaagcaaaccttctggctgtgctcacggagaccttggattccctccctgtggacgaggacggtcttcctagctttgacgcgctcacagatggagatgttacaacagagaatgaagcctccccttcaagtatgccagatggaactccacccccgcaagaagctgaggaaccttccctgcttaagaaactgctgttggctccggcaaacacacaactctcatataatgaatgctctgggctgtcaacccaaaaccatgcgaatcataaccacagaattaggacaaatccagcggtcgtgaagacggaaaattcctggtctaataaggcaaagtctatatgtcaacaacagaagccgcagagaaggccctgttctgaactgcttaaatatcttaccacgaacgacgaccccccacacacgaagcccactgaaaaccgaaattcttcaagggataaatgtacctctaaaaaaaaggcccatacacaatcccagactcaacatttgcaagctaaaccgactacgctttctcttcccctcactccggaaagccccaacgaccccaaaggttccccatttgagaataaaacgatagaacgcaccctgtccgtcgagttgtcaggtacggccggccttactccgcctactactccacctcataaggcgaaccaagataatcccttcagagcttcaccaaagcttaagccaagttgcaaaactgtcgtcccccctccgtctaagaaagcccgctattccgaaagttcatgcacccaaggctctaactctaccaaaaaggggcctgaacagtcagaattgtatgcacagttgagtaaaacgagcgttctcacctctggacatgaggaacgaaaggctaagcggccctcccttaggttgttcggggaccatgactactgccagtccataaattccaaaacagaaatacttgtgtccacttcacaagagcttcatgactctagacaacttgaaaacaaggatgctccttcaagcaatggtcccgggcagattcacagttctaccgacagcgacccctgttatcttagagagacagcggaggttagtaggcaggtgtcccccggatcaacacgaaaacagctccaagaccaggaaatacgagcggagctgaataaacactttggccacccgagccaagctgtcttcgacgacaaagccgataaaactagcgagctgagggatagcgacttctctaatgaacaattctctaaacttccaatgttcataaatagcggcctcgctatggacggcctcttcgatgattccgaggacgaatctgacaagttgaatagtccctgggacgggacccaaagttactccctgttcgatgtgtcaccctcatgttcctcattcaactccccgtgtagagactccgtctcccctccaaaaagcctttttagtcagcggcctcaaaggatgcgatctcgcagtcgatcctttagtaggcacagaagttgttcaagatctccatattctcggtcacggtctagaagtcctggttccagaagttcatccagatcatgttactactacgaaagcggacattgtaggcacaggacccaccggaattctccactgtgcgctagtcgaagtcgatctcctcatagcagacggccacgatatgactcctacgaagagtaccagcacgagcggttgaagcgggaagagtaccggcgcgagtacgaaaaaagggaaagcgaaagagccaaacagcgcgagaggcagagacaaaaagcgattaatcaacaggtcagtagccatttccctctgcaagaggaacgcagggtgatttatgtcgga

SEQ ID NO: 11 - protein, 300 aa, Bos taurus
MSVGPACPQSLLGPEASNSRESSPMPEESYVSLQTSSADTLDTDTVSPLPSSMDLLIQDSPDSSTSPRVKPLSPSVEESTEKEETVPVKKQKIRTVFSQTQLCVLNDRFQRQKYLSLQQMQELSNILNLSYKQVKTWFQNQRMKCKKWQKNNWPRNSNGMPQGPAMAEYPGFYSYHQGCLVNSPGNLPMWGNQTWNNPTWSNQSWNSQSWSNHSWNSQAWCPQAWNNQPWNNQFNNYMEEFLQPGIQLQQNSPVCDLEATLGTAGENYNVIQQTVKYFNSQQQITDLFPNYPLNIQPEDL

SEQ ID NO: 12 - DNA, 903 bp, synthetic construct
  source 1..903: mol_type = other DNA
atgagcgtggggccggcttgccctcaatctctgcttggcccggaggcctctaatagccgtgagagcagtcctatgcctgaggaaagttatgtatccctgcaaacctccagtgccgatacgttggacaccgataccgtgtcacccctgccatcttcaatggaccttctgattcaagactcacccgatagctccaccagtccccgcgtgaagcccctgtccccaagcgttgaagagagcaccgagaaggaagagacagtccctgtgaaaaagcagaagatccggactgttttcagccagacgcaattgtgcgtgttgaacgaccgcttccagcgtcagaagtacctgagcctgcaacagatgcaggagctgagtaacatcctcaacctgtcctacaaacaggtcaagacctggttccagaatcaacggatgaagtgtaagaaatggcaaaagaataactggccccgtaactccaatgggatgccccaaggtcccgctatggcagaatacccagggttctactcctaccaccagggctgcctggtaaacagccctggcaacctccctatgtggggcaaccagacttggaataaccctacatggtctaatcagagttggaactctcagagctggtctaatcactcttggaactcacaagcctggtgccctcaagcttggaataaccagccatggaataaccagttcaataactacatggaagagttcctgcagccaggtattcagctgcaacagaacagccccgtgtgtgacctggaggccaccctgggaaccgccggcgagaactataatgtgatccaacagaccgtcaagtatttcaattctcagcaacagatcacggacctgttcccgaactaccctctgaacatccagcctgaggacttgtaa

SEQ ID NO: 13 - protein, 506 aa, Bos taurus
MADAEVLILPKKHKKKKERKSLPEEAVAEIQHAEEFLIKPESRVAQLDTSQWPLLLKNFDKLNVRTTHYTPLPCGSNPLKREIGDYIRTGFINLDKPSNPSSHEVVAWIRRILRVEKTGHSGTLDPKVTGCLIVCIERATRLVKSQQSAGKEYVGIVRLHNAIEGGTQLSRALETLTGALFQRPPLIAAVKRQLRVRTIYESKMIEYDPERRLGIFWVSCEAGTYIRTLCVHLGLLLGVGGQMQELRRVRSGVMSEKDHMVTMHDVLDAQWLYDNHKDESYLRRVVYPLEKLLTSHKRLVMKDSAVNAICYGAKIMLPGVLRYEDGIEVNQEIVVITTKGEAICMAIALMTTAVISTCDHGIVAKIKRVIMERDTYPRKWGLGPKASQKKLMIKQGLLDKHGKPTDSTPATWMQEYVDYSNSAKKDVPPKAVKATPVVAEVVKTPKRKRESESEDTSPAAPQVVKKEKKKKKKEKAKAAAEKPGAGDSDSTKKKKKKKIKAEMVSE

SEQ ID NO: 14 - DNA, 1521 bp, synthetic construct
  source 1..1521: mol_type = other DNA
atggcagatgctgaagttcttattctccctaagaaacacaaaaagaaaaaagaacgcaaatcactcccagaggaggccgttgcagagatacaacatgcggaagagttcctgatcaaaccagaatctagagttgctcaactggatacatctcaatggcctttgctgctgaaaaatttcgataaactcaacgtcagaacaacccactataccccgctcccgtgtgggtctaatccgcttaaacgagaaatcggtgactacattcggacaggttttattaatctcgataagcctagcaacccctctagtcacgaggttgtcgcttggatccgaaggatattgagggtcgagaagacagggcactcaggtacccttgatcccaaagtcaccggctgcctcatagtttgcatcgaaagagccactcgactggttaaatcccaacaatctgcaggaaaagagtatgttggcatagtcaggcttcataatgcgattgagggtggcacccaactctccagggcactcgaaacacttacgggggcacttttccagcgaccacctcttatagcggcagtcaaacggcaactccgagttaggacgatatacgagagcaagatgatcgagtatgatcctgagagacggcttggtatcttttgggttagttgtgaggccggcacgtatatccgaacgctttgtgtgcacttggggttgctgcttggggtcgggggccaaatgcaggaattgcggcgggtgcggtcaggagtgatgtcagagaaggaccatatggtgacgatgcatgacgtcctcgatgcccagtggctttacgacaatcacaaagatgagtcataccttaggcgagttgtctacccactcgaaaagttgctcacttcccacaaacgacttgtgatgaaagatagcgccgtgaacgcgatctgctatggcgctaagataatgctccccggtgtcctcagatacgaagacgggattgaagttaatcaagaaatcgttgtcattacgactaaaggagaggccatttgcatggcgatcgccctgatgacgactgccgtcataagtacatgcgatcatggaatagtggcaaaaatcaagagggtcatcatggaaagggacacatacccccggaaatggggtctgggaccgaaagcctctcaaaagaaactgatgattaaacagggacttcttgacaaacatggtaaaccgactgatagcactcccgctacatggatgcaagaatatgtggactattctaattccgctaaaaaggatgtgccgcctaaagcagtgaaagcgacccccgtcgttgctgaagtggttaaaacgccaaaacgcaaaagagagtctgaatcagaagacacttctcccgcagcgccgcaggtcgtcaaaaaggagaagaaaaaaaagaagaaagagaaggcgaaagctgccgctgagaagccgggtgccggtgacagcgatagtacaaagaagaaaaaaaaaaagaaaatcaaggctgaaatggtttctgagtag

SEQ ID NO: 15 - protein, 507 aa, Bos taurus
MDPGPPPPQPAPQGQGPPPAQAPQGQGPPSAPGQPAPPGPPAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAAAPAAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVLGGGSSNQQQQMRLQQLQMEKERLRLKQQELLRQVRPQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 16 - DNA, 1524 bp, synthetic construct
  source 1..1524: mol_type = other DNA
atggatcctggaccgcctcccccgcagcctgcgccccagggacaggggccacccccagcgcaggccccacaaggccagggtcccccaagcgctccaggccagcctgcaccacccggccctcccgctgcaccccaggccccacctgccggtcaccagatcgtccacgtccgcggggactccgagacagatctggaagctctctttaacgcggtgatgaacccaaagacggcaaatgtgcctcagaccgtccctatgcgcctgaggaagctgcccgactcctttttcaaacctccggagccaaaatcccattctcgccaggcctccacggatgccggcaccgcgggcgcactgacgccccagcacgtcagggctcattctagccctgccagccttcagctcggcgctgtgagccccgggaccctgacccctacgggagttgtgtccggccccgccgcagcccctgccgcacagcacctgcgccaatcctcttttgagattcccgatgacgtgcctctgcccgccggttgggagatggctaagacctctagcggccagaggtacttcctcaatcacattgaccagactaccacttggcaggacccccggaaggcgatgctttctcagatgaacgtgacagcgccgacttcccctcccgtccaacagaacatgatgaactccgccagcggcccactccccgatgggtgggagcaggctatgacccaggacggtgagatttattacatcaaccacaagaacaagacgacctcttggctggatccaagattggatccgcgcttcgccatgaaccagagaatctcacagtccgcccctgtgaagcagccgcccccgttggcgccgcaaagccctcagggcggagtgctgggcggtggatcttccaaccagcaacagcaaatgcgcttgcagcaattgcagatggaaaaagagaggctgcgcctcaaacaacaggaacttctgaggcaggtgcgcccccaggccatgaggaacatcaacccgtccaccgctaattctcctaagtgccaggaactggctctgcgctctcagctgcccacgctggagcaggacggcgggacccagaatcctgttagttctccgggaatgagccaagaactgcgtactatgactacaaacagctccgacccattcttgaacagcggcacctaccactctcgtgatgagagcaccgactcaggattgtccatgagctcctatagcgtgcctcgtacgcccgatgacttcctcaacagcgtggacgaaatggataccggtgacaccatcaaccaaagcaccctcccaagccagcaaaaccgtttcccagactacttggaggcaatcccggggacaaacgtggatcttggaactctggagggcgacgggatgaacatcgagggcgaagagctcatgccttccctccaggaggcgctgagctccgacatcctgaacgatatggaatccgtgttggctgcaaccaagctggacaaggagtccttcttgacatggttgtga

SEQ ID NO: 17 - DNA, 1051 bp, synthetic construct
  source 1..1051: mol_type = other DNA
gatgctgcagttcctcattagcacggtcctcacgcagcaccgcacggcctgctgggccgggcccggccgggagcctcagagcggggccgttgggcggccgtgcaccccggtatcgcggtgcggggacggatatgggacccagcgctccccagcagctcggggctgcgcggccaacacgcgcaagggctgaggacggggagcgggcaggctgcagcctcgcatcgcgctctttgtcccgcccgcggccagacgccgttctctgggtacccgcaggagcgcaatccgcacccactgtaccgcatcgcgtgatgcagccgcatcgccccggcctcgtccgaggacgccacggccccacgtggggctttgtctgcacgcagctccgcgccccccggccccggggcacccctcgccgcccccaccccgcacgggcaccgtggtacggcagcaaccccctaagcccaccctgagcccaacggccgccctttgttgcgaggggggagggggaaggagaggagagggggggccgtttcgcccgcaccgtgcgcgcacgtagccccgagcccgcccccgcgctgccattcggcgctggcccggcggggcggggcgcggggcgctgcctccgtgtctataaagggcggcggcgcggcactgcgccaccttctcactgcgcgctggggccgttgacgtgcagcaggtaacgggcggggggacgtgggggcggagaagcgtaccccccacccacccgcctgtctgcctgcgcccccctgtaggggttggggctgtaggaccgcgaggagtgcggcgggggaaggggggagggggtctccgcgcaggaccgcgtggtgcccggggcacaaaggcgggggcgggcggcgcgagatgaagggggtgggggttgggggagagagggagaactgcgggggagagagctcgatggggatgaaggaagggatggcgggtcggggggcggtgcccagagctccgcagcgcgcgcggtggggggacgttactgggccggcggtctgcgtgcggcaccgcacgtctcactgcc

(19) United States
(12) Patent Application Publication
(10) Pub. No.: US 2024/0200033 A1
(43) Pub. Date: Jun. 20, 2024

(54) COMPOSITIONS AND METHODS FOR EXTENDING THE REPLICATIVE CAPACITY OF BOVINE CELLS

(71) Applicant: Upside Foods, Inc., Berkeley, CA (US)

(72) Inventors: Carmen Banks, San Francisco, CA (US); Daphne Dambournet, Paris (FR)

(21) Appl. No.: 18/544,063

(22) Filed: Dec. 18, 2023

Related U.S. Application Data
(60) Provisional application No. 63/387,854, filed on Dec. 16, 2022.

(51) Int. Cl. C12N 5/077; A23L 13/40; C12N 9/12; C12N 15/85

(52) U.S. Cl. CPC C12N 5/0658; A23L 13/48; C12N 9/1276; C12N 15/85; C12N 2501/40

(57) ABSTRACT

Provided herein are methods of method for extending the replicative capacity of a bovine cell. For example, a method for extending the replicative capacity of a bovine cell includes (a) isolating a population of cells from bovine tissue; (b) introducing into a cell of the population of cells a polynucleotide encoding telomere reverse transcriptase (TERT); (c) introducing into the cell of step (b) at least a first polynucleotide encoding a first protein that is capable of modulating cell proliferation; and (d) culturing the population of bovine cells in a cultivation infrastructure is provided herein.

### 0. SEQUENCE LISTING

A Sequence Listing XML is submitted herewith as an xml file named 38279_0013U2_SEQLIST.xml, created on Dec. 18, 2023, having a size of 31,144 bytes, and is hereby incorporated by reference.

### 1. BACKGROUND OF THE INVENTION

The mass production of cells remains limited by several factors, thus limiting final yields. Mass production of cells finds several downstream applications. For example, foods formulated from metazoan cells, cultured in vitro, have prospective advantages over their corporal-derived animal counterparts, including improved nutrition and safety. Production of these products have been projected to require fewer resources, convert biomass at a higher caloric efficiency and result in reduced environmental impacts relative to conventional in vivo methods. Together, metazoan cells, and their extracellular products, constitute a biomass that can potentially be harvested from a cultivation infrastructure for formulation of cell-based food products, such as cultured meat.

However, mass production of cells originating from cultured metazoan cells remains limited by several factors, for example, by senescence of the cells prior to reaching a density or number needed for a cell-based meat product, thus driving up the cost and resources needed to produce cell-based meat products. Provided herein are compositions and methods that address this and other related needs.

### 2. SUMMARY OF THE INVENTION

This disclosure is based in part on the finding that despite being sufficient to increase replicative capacity in in vitro cultures of other species (e.g., chicken), introducing TERT alone into bovine fibroblast cells fails to increase replicative capacity. Applicant found that introducing into bovine cells a polynucleotide encoding telomere reverse transcriptase (TERT) and at least one polynucleotide encoding a protein that is capable of modulating cell proliferation increases a bovine cells replicative capacity, thereby increasing population doubling levels and reducing the number of starting cells needed in order to produce cell-based meat products. Additionally, Applicant found that using non-integrating methods for introducing of one or both of the polynucleotides encoding TERT and the at least one polynucleotide encoding a protein that is capable of modulating cell proliferation was sufficient to increase replicative capacity of the bovine cells and, in embodiments, reduce the doubling time of the bovine cells. The methods described herein further maintain the increased replicative capacity of the bovine cells after multiple doublings (including at least 50, 60, or 70) after the expression of the TERT and at least one polynucleotide encoding a protein that is capable of modulating cell proliferation

Overall, this work demonstrated the ability to increase (i.e., expand) replicative capacity of bovine cells such that adequate numbers can be generated and thereby allow production of cell-based meats suitable for consumption. These findings are important because manufacturing cells suitable for consumption requires cell line adaption into particular culture formats (i.e., suspension) and cell culture media that require vast amounts of bovine cells, which is not feasible given the methods available in the art. The engineered cells provided enable PDL greater than 100-thereby increasing the supply of bovine cells that can be used to produce the cell based meat products described herein.

### 2.1 EXEMPLARY EMBODIMENTS

Embodiment 1. A method for extending the replicative capacity of a bovine cell, comprising (a) isolating a population of cells from bovine tissue, (b) introducing into a cell of the population of cells a polynucleotide encoding telomere reverse transcriptase (TERT) (c) introducing into the cell of step (b) at least a first polynucleotide encoding a first protein that is capable of modulating cell proliferation, wherein both the TERT and first protein are overexpressed in said cell, and (d) culturing the population of bovine cells in a cultivation infrastructure.

Embodiment 2. A method for extending the replicative capacity of a bovine cell, comprising (a) isolating a population of cells from bovine tissue, (b) incorporating into the genome of a cell of the population of cells a polynucleotide encoding telomere reverse transcriptase (TERT), (c) introducing into the cell of step (b) at least a first polynucleotide encoding a first protein that is capable of modulating cell proliferation, wherein both the TERT and first protein are overexpressed in said cell, and (d) culturing the monoculture of bovine cells in a cultivation infrastructure.

Embodiment 3. A method for extending the replicative capacity of a bovine cell, comprising (a) isolating a population of cells from bovine tissue, (b) introducing into a cell of the population of cells a polynucleotide encoding telomere reverse transcriptase (TERT), (c) incorporating into the genome of the cell of step (b) at least a first polynucleotide encoding a first protein that is capable of modulating cell proliferation, wherein both the TERT and first protein are overexpressed in said cell, and (d) culturing the monoculture of bovine cells in a cultivation infrastructure.

Embodiment 4. A method for extending the replicative capacity of a bovine cell, comprising (a) isolating a population of cells from bovine tissue, (b) incorporating into the genome of a cell of the population of cells a polynucleotide encoding telomere reverse transcriptase (TERT), (c) incorporating into the genome the cell of step (b) at least a first polynucleotide encoding a first protein that is capable of modulating cell proliferation, wherein both the TERT and first protein are overexpressed in said cell, and (d) culturing the monoculture of bovine cells in a cultivation infrastructure.

Embodiment 5. A method for improving differentiation capacity of a bovine cell line, comprising (a) isolating a population of cells from bovine tissue, (b) introducing into, or incorporating into the genome of, a cell of the population of cells a polynucleotide encoding telomere reverse transcriptase (TERT), (c) introducing into, or incorporating into the gnome of, the cell of step (b) at least a first polynucleotide encoding a first protein that is capable of modulating cell proliferation, wherein both the TERT and first protein are overexpressed in said cell, (d) culturing the monoculture of bovine cell in a cultivation infrastructure, and (e) inducing myogenic-specific differentiation.

Embodiment 6. A method for making cell-based meat suitable for consumption, comprising: (a) isolating a population of cells from bovine tissue, (b) introducing into, or incorporating into the genome of, a cell of the population of cells a polynucleotide encoding telomere reverse transcriptase (TERT), (c) introducing into, or incorporating into the genome of, the cell of step (b) at least a first polynucleotide encoding a first protein that is capable of modulating cell proliferation, (d) inducing myogenic specific differentiation, wherein the differentiated cells form myocytes and multinucleated myotubes, and (e) culturing the myocytes and myotubes to generate skeletal muscle fibers, thereby producing a cultured edible product.

Embodiment 7. The method of any one of embodiments 2-6, further comprising integrating at least a first integrase recognition site into the genome of the bovine cell prior to step (b) or step (c) or concurrent with step (b) or step (c).

Embodiment 8. The method of any one of embodiments 2 or 4-7, wherein incorporating into the genome the polynucleotide encoding TERT is effected by introducing into the cell: (i) the polynucleotide encoding TERT and (ii) an integrase or a polynucleotide encoding an integrase, whereby the polynucleotide encoding TERT is incorporated into the genome by the integrase at the first integrase recognition site integrated into the genome.

Embodiment 9. The method of any one of embodiments 3-7, wherein incorporating into the genome the first polynucleotide encoding the first protein that is capable of modulating cell proliferation is effected by introducing into the cell: (i) the first polynucleotide encoding the first protein that is capable of modulating cell proliferation and (ii) an integrase or a polynucleotide encoding an integrase, whereby the first polynucleotide encoding the first protein is incorporated into the genome by the integrase at the first integrase recognition site integrated into the genome.

Embodiment 10. The method of any one of embodiments 2-6, wherein incorporating into the genome the polynucleotide encoding TERT is effected by a method selected from: viral vector-mediated integration, non-viral vector mediated integration, CRISPR/Cas9-mediated integration, zinc-finger nuclease-mediated integration, and TALEN-mediate integration.

Embodiment 11. The method of any one of embodiments 2-6, wherein incorporating into the genome the first polynucleotide encoding the first protein that is capable of modulating cell proliferation is effected by a method selected from: viral vector-mediated integration, non-viral vector mediated integration, CRISPR/Cas9-mediated integration, zinc-finger nuclease-mediated integration, and TALEN-mediate integration.

Embodiment 12. The method of any one of embodiments 1-3 or 5-7, wherein introducing into the cell the polynucleotide encoding TERT, the first polynucleotide encoding the first protein capable of modulating cell proliferation, or both, is effected such that the introduced polynucleotide(s) do not integrate into the genome of the cell.

Embodiment 13. The method of any one of embodiments 1-6, wherein steps (b) and (c) are performed concurrently.

Embodiment 14. The method of any one of embodiments 1-13, wherein the polynucleotide encoding TERT and the first polynucleotide encoding the first protein that is capable of modulating cell proliferation are on the same contiguous polynucleotide.

Embodiment 15. The method of any one of embodiments 1-14, wherein the population of bovine cells comprising the polynucleotide encoding TERT and the first polynucleotide encoding the first protein that is capable of modulating cell proliferation have higher proliferation rates as compared to a bovine cell not containing the polynucleotide encoding TERT and the at least one polynucleotide encoding the first protein that is capable of modulating cell proliferation.

Embodiment 16. The method of one of embodiments 1-14, wherein the population of bovine cells comprising the polynucleotide encoding TERT and the first polynucleotide encoding the first protein that is capable of modulating cell proliferation has a population doubling level (PDL) at least 1.5-fold greater than a bovine cell not containing the polynucleotide encoding TERT and the at least one polynucleotide encoding the first protein that is capable of modulating cell proliferation.

Embodiment 17. The method of any one of embodiments 1-14, wherein the population of bovine cells comprising the polynucleotide encoding TERT and the first polynucleotide encoding the first protein that is capable of modulating cell proliferation has a population doubling level (PDL) at least 2-fold greater than a bovine cell not containing the polynucleotide encoding TERT and the at least one polynucleotide encoding the first protein that is capable of modulating cell proliferation.

Embodiment 18. The method of any one of embodiments 1-14, wherein, after introducing (or incorporating) the polynucleotide encoding TERT and the first polynucleotide encoding the first protein that is capable of modulating cell proliferation, the population of bovine cells have a population doubling level (PDL) of at least 60.

Embodiment 19. The method of any one of embodiments 1-14, wherein, after introducing (or incorporating) the polynucleotide encoding TERT and the first polynucleotide encoding the first protein that is capable of modulating cell proliferation, the population of bovine cells have a population doubling level (PDL) of at least 70.

Embodiment 20. The method of any one of embodiments 1-14, wherein, after introducing (or incorporating) the polynucleotide encoding TERT and the first polynucleotide encoding the first protein that is capable of modulating cell proliferation, the population of bovine cells have a population doubling level (PDL) of at least 80.

Embodiment 21. The method of any one of embodiments 1-14, wherein, after introducing (or incorporating) the polynucleotide encoding TERT and the first polynucleotide encoding the first protein that is capable of modulating cell proliferation, the population of bovine cells have a population doubling level (PDL) of at least 90.

Embodiment 22. The method of any one of embodiments 1-14, wherein after introducing (or incorporating) the polynucleotide encoding TERT and the first polynucleotide encoding the first protein that is capable of modulating cell proliferation, the population of bovine cells have a population doubling level (PDL) of at least 100.

Embodiment 23. The method of any one embodiments 1-22, wherein the protein that is capable of modulating cell proliferation is selected from BMI-1, CDK4, Cyclin D1, PCG1α, Nanog, DCK1, and YAP.

Embodiment 24. The method of embodiment 23, wherein the protein that is capable of modulating cell proliferation is a BMI-1 protein.

Embodiment 25. The method of embodiment 24, wherein the BMI-1 protein has at least 80% sequence identity to an amino acid sequence of SEQ ID NO: 3.

Embodiment 26. The method of embodiment 23, wherein the protein that is capable of modulating cell proliferation is a CDK4 protein.

Embodiment 27. The method of embodiment 26, wherein the CDK4 protein has at least 80% sequence identity to an amino acid sequence of SEQ ID NO: 5.

Embodiment 28. The method of embodiment 23, wherein the protein that is capable of modulating cell proliferation is a Cyclin D1 protein.

Embodiment 29. The method of embodiment 28, wherein the Cyclin D1 protein has at least 80% sequence identity to an amino acid sequence of SEQ ID NO: 7.

Embodiment 30. The method of embodiment 23, wherein the protein that is capable of modulating cell proliferation is a PGC1α protein.

Embodiment 31. The method of embodiment 30, wherein the PGC1α protein has at least 80% sequence identity to an amino acid sequence of SEQ ID NO: 9.

Embodiment 32. The method of embodiment 23, wherein the protein that is capable of modulating cell proliferation is a Nanog protein.

Embodiment 33. The method of embodiment 32, wherein the Nanog protein has at least 80% sequence identity to an amino acid sequence of SEQ ID NO: 11.

Embodiment 34. The method of embodiment 23, wherein the protein that is capable of modulating cell proliferation is a YAP protein.

Embodiment 35. The method of embodiment 34, wherein the YAP protein has at least 80% sequence identity to an amino acid sequence of SEQ ID NO: 15.

Embodiment 36. The method of embodiment 23, wherein the protein that is capable of modulating cell proliferation is a DKC1.

Embodiment 37. The method of embodiment 36, wherein the DKC1 protein has at least 80% sequence identity to an amino acid sequence of SEQ ID NO: 13.

Embodiment 38. The method of any one of embodiments 1-37, further comprising selecting the cells following step (b), step (c), or both.

Embodiment 39. The method of any one of embodiments 1-38, wherein the selecting comprises physical, mechanical, or chemical selection.

Embodiment 40. The method of embodiment 39, wherein selecting comprises physical separation.

Embodiment 41. The method of embodiment 39, wherein selecting comprises mechanical separation.

Embodiment 42. The method of embodiment 39, wherein selecting comprises chemical selection.

Embodiment 43. The method of any one of embodiments 1-42, further comprising introducing into, or incorporating into the genome of, the cell a polynucleotide encoding a selectable marker.

Embodiment 44. The method of embodiment 43, wherein the polynucleotide encoding the selectable marker is on the same polynucleotide containing the polynucleotide encoding TERT.

Embodiment 45. The method of embodiment 43, wherein the polynucleotide encoding the selectable marker is on the same polynucleotide containing the at least first polynucleotide encoding the first protein that is capable of modulating cell proliferation.

Embodiment 46. The method of any one of embodiments 43-45, wherein the selectable marker is an antibiotic resistance protein that confers antibiotic resistance when expressed.

Embodiment 47. The method of embodiment 46, wherein the antibiotic resistance protein is a puromycin resistance protein.

Embodiment 48. The method of embodiment 47, wherein chemical selection comprises contacting the cells with an antibiotic.

Embodiment 49. The method of any one of embodiments 43-45, wherein the selectable marker is a fluorophore.

Embodiment 50. The method of embodiment 49, wherein selecting comprises performing fluorescence activated cell sorting (FACS).

Embodiment 51. The method of any one of embodiments 2-50, further comprising reducing or eliminating expression of TERT, expression of the at least first protein that is capable of modulating cell proliferation, or both.

Embodiment 52. The method of embodiment 51, wherein reducing or eliminating expression is effected by introducing into the cell an effector protein.

Embodiment 53. The method of embodiment 51, wherein the effector protein disrupts expression of the polynucleotide encoding TERT.

Embodiment 54. The method of embodiment 52 or 53, wherein the effector protein disrupts expression of the polynucleotide encoding TERT at the genomic location at which the polynucleotide encoding TERT is incorporated.

Embodiment 55. The method of any one of embodiments 52-54, wherein the effector protein disrupts expression of the at least first polynucleotide encoding the first protein that is capable of modulating cell proliferation.

Embodiment 56. The method of any one of embodiments 52-54, wherein the effector protein disrupts expression of the at least first polynucleotide encoding the first protein that is capable of modulating cell proliferation at the genomic location at which the polynucleotide encoding a protein that is capable of modulating cell proliferation is incorporated.

Embodiment 57. The method of any one of embodiments 52-56, wherein the effector protein is selected from a nuclease, recombinase, and integrase.

Embodiment 58. The method of any one of embodiments 52-57, wherein the effector protein is a nuclease.

Embodiment 59. The method of embodiment 58, wherein the nuclease induces a genetic perturbation in the integrated polynucleotide, whereby the genetic perturbation disrupts expression of the protein encoded by the polynucleotide.

Embodiment 60. The method of embodiment 58 or 59, wherein the nuclease is Cas9 endonuclease.

Embodiment 61. The method of any one of embodiments 52-57, wherein the effector protein is a recombinase.

Embodiment 62. The method of embodiment 61, wherein the recombinase excises, inverts, or translocates, or a combination thereof, the integrated polynucleotide, whereby excising, inverting, or translocating disrupts expression of the protein encoded by the polynucleotide.

Embodiment 63. The method of embodiments 61 or 62, wherein the recombinase is a CRE protein.

Embodiment 64. The method of any one of embodiments 52-57, wherein the effector protein is an integrase.

Embodiment 65. The method of embodiment 64, wherein the integrase excises, inverts, or translocates, or a combination thereof, the integrated polynucleotide, whereby excising, inverting, or translocating disrupts expression of the protein encoded by the polynucleotide.

Embodiment 66. The method of embodiment 64 or 65, wherein the integrase is PhiC31.

Embodiment 67. The method of any one of embodiment 51-66, wherein the polynucleotide encoding TERT comprises recognition sites flanking the polynucleotide sequence encoding TERT.

Embodiment 68. The method of any one of embodiments 51-66, wherein the at least first polynucleotide encoding the first protein that is capable of modulating cell proliferation comprises recognition sites flanking the polynucleotide sequenced encoding the protein that is capable of modulating cell proliferation.

Embodiment 69. The method of embodiment 67 or 68, wherein the recognition sites are selected from lox66, lox71, attB, attP, attL, attR, and FRT.

Embodiment 70. The method of embodiment 69, wherein the recognition site is a lox site.

Embodiment 71. The method of embodiment 70, wherein the lox site is selected from lox66 and lox71.

Embodiment 72. The method of embodiment 71, wherein the lox site is a lox66.

Embodiment 73. The method of embodiment 71, wherein the lox site is a lox71.

Embodiment 74. The method of any one of embodiments 51-73, wherein the effector protein is CRE, thereby disrupting expression by excising, inverting, or translocating a polynucleotide sequence flanked by the loxP site.

Embodiment 75. The method of any one of embodiments 51-74, wherein disrupting expression at the site of genome incorporation comprises disruption of a portion of the incorporated polynucleotide.

Embodiment 76. The method of any one of embodiments 1-75, wherein the bovine tissue comprises skin, muscle, or a combination thereof.

Embodiment 77. The method of any one of embodiments 1-75, wherein the bovine cells are non-myogenic cells.

Embodiment 78. The method of embodiment 76, wherein the non-myogenic cells are selected from: fibroblasts, mesenchymal cells, and chondrocytes.

Embodiment 79. The method of any one of embodiments 1-75, wherein the bovine cells are myogenic cells.

Embodiment 80. The method of embodiment 79, wherein the myogenic cells are myoblasts, myocytes, satellite cells, side population cells, muscle derived stem cells, myofibroblasts, mesenchymal stem cells, mesenchymal progeny, myogenic pericytes, mesoangioblasts, or endoderm or ectoderm lineage cells having been transdifferentiated into the myogenic cells.

Embodiment 81. A population of bovine cells suitable for consumption produced by any of the methods of embodiments 1-80.

Embodiment 82. A population of immortalized bovine cells suitable for consumption produced by any of the methods of embodiments 1-80.

Embodiment 83. A non-myogenic cell, a myogenic cell, or a population thereof suitable for consumption comprising (a) a polynucleotide encoding TERT and (b) a polynucleotide encoding a protein that is capable of modulating cell proliferation.

Embodiment 84. The non-myogenic cell, myogenic cell, or population thereof of embodiment 83, wherein the protein that is capable of modulating cell proliferation is selected from BMI-1, CDK4, Cyclin D1, PCG1α, Nanog, DKC1, and YAP.

Embodiment 85. The non-myogenic cell, myogenic cell, or population thereof of embodiment 84, wherein the protein that is capable of modulating cell proliferation is a BMI-1 protein.

Embodiment 86. The non-myogenic cell, myogenic cell, or population thereof of embodiment 85, wherein the BMI-1 protein has at least 80% sequence identity to an amino acid sequence of SEQ ID NO: 3.

Embodiment 87. The non-myogenic cell, myogenic cell, or population thereof of embodiment 84, wherein the protein that is capable of modulating cell proliferation is a CDK4 protein.

Embodiment 88. The non-myogenic cell, myogenic cell, or population thereof of embodiment 87, wherein the CDK4 protein has at least 80% sequence identity to an amino acid sequence of SEQ ID NO: 5.

Embodiment 89. The non-myogenic cell, myogenic cell, or population thereof of embodiment 84, wherein the protein that is capable of modulating cell proliferation is a Cyclin D1 protein.

Embodiment 90. The non-myogenic cell, myogenic cell, or population thereof of embodiment 89, wherein the Cyclin D1 protein has at least 80% sequence identity to an amino acid sequence of SEQ ID NO: 7.

Embodiment 91. The non-myogenic cell, myogenic cell, or population thereof of embodiment 84, wherein the protein that is capable of modulating cell proliferation is a PGC1α protein.

Embodiment 92. The non-myogenic cell, myogenic cell, or population thereof of embodiment 91, wherein the PGC1α protein has at least 80% sequence identity to an amino acid sequence of SEQ ID NO: 9.

Embodiment 93. The non-myogenic cell, myogenic cell, or population thereof embodiment 84, wherein the protein that is capable of modulating cell proliferation is a Nanog protein.

Embodiment 94. The non-myogenic cell, myogenic cell, or population thereof of embodiment 93, wherein the Nanog protein has at least 80% sequence identity to an amino acid sequence of SEQ ID NO: 11.

Embodiment 95. The non-myogenic cell, myogenic cell, or population thereof of embodiment 84, wherein the protein that is capable of modulating cell proliferation is a YAP protein.

Embodiment 96. The non-myogenic cell, myogenic cell, or population thereof of embodiment 95, wherein the YAP protein has at least 80% sequence identity to an amino acid sequence of SEQ ID NO: 15.

Embodiment 97. The non-myogenic cell, myogenic cell, or population thereof of embodiment 84, wherein the protein that is capable of modulating cell proliferation is a DKC1.

Embodiment 98. The non-myogenic cell, myogenic cell, or population thereof of embodiment 97, wherein the DKC1 protein has at least 80% sequence identity to an amino acid sequence of SEQ ID NO: 13.

Embodiment 99. The non-myogenic cell, myogenic cell, or population thereof of any one of embodiments 83-98, wherein the polynucleotide encoding TERT comprises recognition sites flanking the polynucleotide sequence encoding TERT.

Embodiment 100. The non-myogenic cell, myogenic cell, or population thereof of any one of embodiments 83-98, wherein the at least first polynucleotide encoding the first protein that is capable of modulating cell proliferation comprises recognition sites flanking the polynucleotide sequenced encoding the protein that is capable of modulating cell proliferation.

Embodiment 101. The non-myogenic cell, myogenic cell, or population thereof of embodiment 99 or 100, wherein the recognition sites are selected from lox66, lox71, attB, attP, attL, attR, and FRT.

Embodiment 102. The non-myogenic cell, myogenic cell, or population thereof of embodiment 101, wherein the recognition site is a lox site.

Embodiment 103. The non-myogenic cell, myogenic cell, or population thereof of claim 102, wherein the lox site is selected from lox66 and lox71.

Embodiment 104. The non-myogenic cell, myogenic cell, or population thereof of embodiment 103, wherein the lox site is a lox66.

Embodiment 105. The non-myogenic cell, myogenic cell, or population thereof of embodiment 103, wherein the lox site is a lox71.

### 3. BRIEF DESCRIPTION OF THE DRAWINGS

These and other features, aspects, and advantages of the present invention will become better understood with regard to the following description, and accompanying drawings, where:

FIG. 1A shows a bright field image of wild type (“WT”) bovine fibroblasts (4C).

FIG. 1B shows a bright field image of bovine fibroblasts following (4C) transduced with TERT. Arrows indicate senescing cells.

FIG. 2 illustrates one non-limiting example workflow of the methods described herein.

FIG. 3 shows a PhiC31 integrase expression plasmid with a ggGAPDH promoter driving expression of a Bos taurus (bt) BMI1.

FIG. 4A shows a PhiC31 integrase expression plasmid with a ggGAPDH promoter driving expression of a bt mutant CDK4.

FIG. 4B shows a PhiC31 integrase expression plasmid with a ggGAPDH promoter driving expression of a bt mutant CDK4 and Cyclin D1, where CDK4 and Cyclin D1 are linked by a T2A.

FIG. 5 shows population doubling level (PDL) for wild type (BT004FC FB) (yellow line), a cell line transduced with TERT and BMI1 (BT 4C btTERT_Bt BMI1) (red line), and a cell line transduced with TERT and “CC”: CDK4 and cyclin D1 (Bt 4C btTERT_Bt CC) (blue line).

FIG. 6 shows a population doubling time (double time (h)) for a cell line transduced with TERT and BMI1 (BT 4C btTERT_Bt BMI1) and a cell line transduced with TERT and “CC”: CDK4 and cyclin D1 (Bt 4C btTERT_Bt CC).

FIG. 7 shows copy number variants (CNV) for each for the transduction conditions.

FIG. 8A shows expression levels (log fold change relative to WT) for each of the transduction conditions at population doubling level (PDL) 30, PDL 48, and PDL 52.

FIG. 8B shows expression levels (log fold change relative to WT) for each of the transduction conditions (BMI1, CDK4, CyclinD1) at population doubling level (PDL) 30, PDL 48, and PDL 52.

FIG. 9A shows expression levels (log fold change relative to WT) for BMI1, CDK4, CyclinD1 for Bovine 4C cells transduced with TERT and CC (CDK4 and CYCLIND1) at population doubling level (PDL) 48, PDL 52, and PDL 75.

FIG. 9B shows expression levels (log fold change relative to WT) for BMI1, CDK4, Cyclin D1 for Bovine 4C cells transduced with TERT and BMI1 at PDL 48, PDL 52, and PDL 75.

FIG. 10 shows a population doubling level (PDL) for Bovine 4C cells transduced with various polynucleotides.

FIG. 11A shows mRNA expression levels for TERT (log fold change relative to WT) in bovine 4C cells following each of the transduction conditions listed on the x-axis (Bt TERT+Bt CKD4; Bt TERT+Bt Nanog; Bt TERT+Bt PGC1a; Bt TERT+Bt YAP; Bt TERT+Bt DKC1; and Bt TERT+Bt CDK4-CyclinD1 (“CC”)). Cells were isolated and mRNA expression was assessed at population doubling levels (PDLs) 25, 45 and 60. * indicates not enough RNA was collected at PDL 25 for the bovine cells transduced with Bt TERT+Bt DKC1 for assessment.

FIG. 11B shows mRNA expression levels for a gene of interest (GOI) (log fold change relative to WT) in bovine 4C cells following each of the transduction conditions listed on the x-axis (Bt TERT+Bt CKD4; Bt TERT+Bt Nanog; Bt TERT+Bt PGC1a; Bt TERT+Bt YAP; Bt TERT+Bt DKC1; and Bt TERT+Bt CDK4-CyclinD1 (“CC”)). Cells were isolated and mRNA expression was assessed at population doubling levels (PDLs) 25, 45, 60, and 75. * indicates not enough RNA was collected for assessment.

FIGS. 12A-12D show mRNA expression levels for endogenous genes (log fold change relative to WT) for bovine 4C cells following each of the transduction conditions listed on the x-axis (Bt TERT+Bt CKD4; Bt TERT+Bt Nanog; Bt TERT+Bt PGC1a; Bt TERT+Bt DKC1; Bt TERT+Bt YAP; Bt TERT+Bt CDK4-CyclinD1 (“CC”)+INT (Integrase); and Bt TERT+Bt CDK4-CyclinD1 (“CC”) (−) INT (Integrase)+No selection). FIG. 12A shows CDK4 endogenous mRNA expression following the various transduction conditions described above. FIG. 12B shows CyclinD1 endogenous mRNA expression following the various transduction conditions described above. FIG. 12C shows Nanog endogenous mRNA expression following the various transduction conditions described above. FIG. 12D shows DKC1 endogenous mRNA expression following the various transduction conditions described above.

FIG. 13 shows copy number (CNV) for bovine cells following the transduction as described in FIGS. 12A-12D. Abbreviation: puro=puromycin, GOI=gene of interest, int=integrase, and Mut=mutant.

FIG. 14 shows a population doubling level (PDL) for bovine 4C cells transduced with Bt TERT+Bt CDK4-CyclinD1 (“CC”)+INT (Integrase) and Bt TERT+Bt CDK4-CyclinD1 (“CC”) (−) INT (Integrase)+No selection as compared to a control (BT 4C ADH57).

FIG. 15 shows copy number (CNV) for bovine cells following the transduction as described in FIG. 14. Copy number was assessed after cells isolated at PDL 70, PDL 85, PDL 92, and PDL 95.

FIG. 16A a PhiC31 integrase expression plasmid with a ggGAPDH promoter driving expression of a bt Nanog.

FIG. 16B a PhiC31 integrase expression plasmid with a ggGAPDH promoter driving expression of a bt PGC1alpha.

FIG. 16C a PhiC31 integrase expression plasmid with a ggGAPDH promoter driving expression of a bt YAP.

FIG. 16D a PhiC31 integrase expression plasmid with a ggGAPDH promoter driving expression of a bt DKC1.

### 4. DETAILED DESCRIPTION OF THE INVENTION

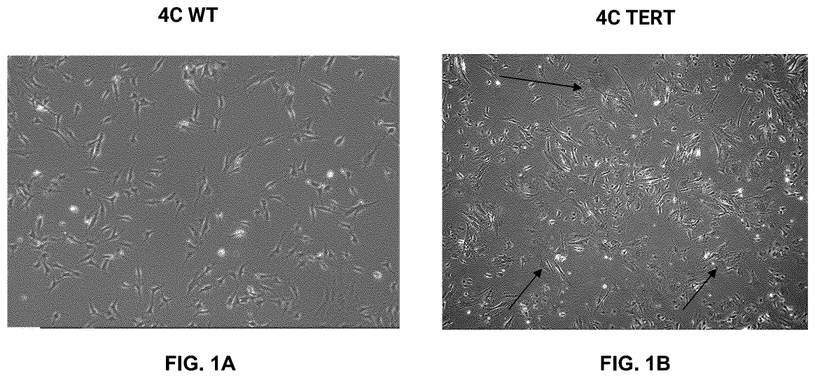

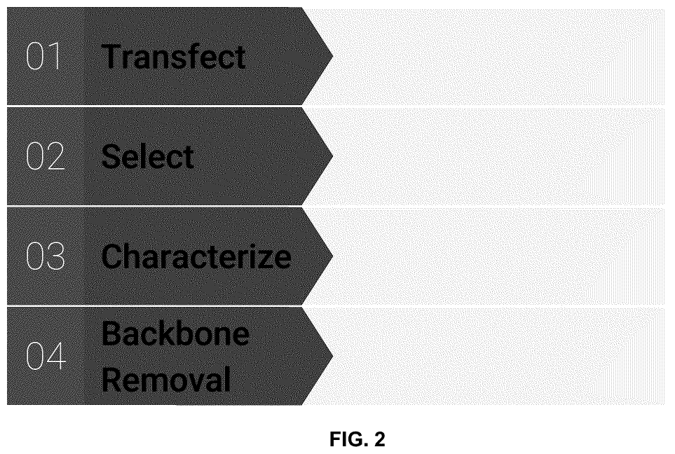

Provided herein are methods for engineering a bovine cell to expand its replication capacity. In particular, this disclosure is based in part on the finding that despite being sufficient to increase replicative capacity in in vitro cultures of other species (e.g., chicken), introducing TERT alone into bovine cells fails to expand replicative capacity (see FIG. 1). Efforts to expand replicative capacity in bovine cells, which are not immortalized, is further complicated by low transfection efficiency compared to other species (e.g., chicken) and issues with backbone removal. This disclosure is also based on the finding that introduction of the polynucleotides encoding TERT and a protein that is capable of modulating cell proliferation by transient transfection and not promoting integration of these polynucleotides into the genome of the bovine cells is sufficient to expand the replicative capacity of the bovine cells and when cultured the bovine cells have PDL that are longer than the unmodified cells. Even after 50 or more doublings, the bovine cells that arose from the modified bovine cells maintain the expanded replicative capacity in that the cells can continue to grow in culture and do not exhibit increased doubling time (or at least maintain a doubling time of approximately 20 hours or less).

In one aspect, provided herein is a method that expands replicative capacity and decreases population doubling times by expressing or increasing the expression in bovine cells of a TERT and at least one protein that is capable of modulating cell proliferation, including, for example, introducing into bovine cells a polynucleotide encoding telomere reverse transcriptase (TERT) and introducing at least one polynucleotide encoding a protein that is capable of modulating cell proliferation, which polynucleotides are under the control of one or more regulatory elements that promote expression of the polynucleotide in the bovine cells or can promote expression under the appropriate conditions, for example, an inducible promoter. Further selection of the bovine cells transduced with both the polynucleotide encoding TERT and the polynucleotide encoding the protein that is capable of modulating cell proliferation, enables cell line generation and also allows for efficient removal of the backbone of the constructs (i.e., the portion of the constructs that does not include the nucleotide sequence encoding the TERT or protein that is capable of modulating cell proliferation or regulatory elements that promote expression) used for cell transduction. In another aspect, provided herein is a method that expands replicative capacity and decreases doubling times of a bovine cell by using non-integrating methods for introducing into a bovine cell of one or both of the polynucleotides encoding TERT and the at least one polynucleotide encoding a protein that is capable of modulating cell proliferation, in embodiments, along with operably linked regulatory elements that promote expression in the bovine cell of the TERT and the protein capable of modulating cell proliferation. By introducing the polynucleotides encoding TERT and the protein capable of modulating cell proliferation transiently, after at least 5, 10, 15 or 20 doublings, the cells do not contain detectable levels of the polynucleotides encoding TERT and the protein capable of modulating cell proliferation or the backbone or vector of the construct containing these polynucleotides and used to introduce the polynucleotides to the bovine cells.

### 4.1. Definitions

Unless defined otherwise, all technical and scientific terms used herein have the same meaning as is commonly understood by one of ordinary skill in the art to which this disclosure belongs. In the event that there is a plurality of definitions for terms cited herein, those in this section prevail unless otherwise stated.

Throughout this disclosure, the term “a” or “an” entity refers to one or more of that entity. As such, the terms “a” (or “an”), “one or more,” and “at least one” can be used interchangeably herein. Furthermore, “and/or” as used in a phase such as “A and/or B” herein is intended to include “A and B,” “A” (alone), and “B” (alone). Likewise, the term “and/or” as used in a phrase such as “A, B, and/or C” is intended to encompass each of the following aspects: A, B, and C; A, B, or C; A or C, A or B; B or C; A and C; A and B; B and C; A (alone); B (alone); and C (alone).

As used herein, the terms “comprising” and “including” or grammatical variants thereof are to be taken as specifying the stated features, integers, steps, or components but do not preclude the addition of one or more additional features, integers, steps, components, or groups thereof. This term encompasses the terms “consisting of” and “consisting essentially of”.

As used herein, the terms “cell” and “cell line” are sometimes used interchangeably. As used herein, the term “cell” can refer to one or more cells originating from a cell line. As used herein, the term “cell line” can refer to a population of cells and can be from the same source or genetically related.

As used herein, the term “myoblast” refers to mononucleated muscle cells. They are embryonic precursors of myocytes, also called muscle cells. Although myoblasts may be classified as skeletal muscle myoblasts, smooth muscle myoblasts, and cardiac muscle myoblasts depending on the type of muscle cell that they will differentiate into, in this specification the term myoblasts refer to skeletal muscle myoblasts.

As used herein, the term “myotube” refers to elongated structures, the result of differentiated myoblast. Upon differentiation, myoblasts fuse into one or more nucleated myotubes and express skeletal muscle markers.

As used herein, the terms “immortalized cell” or “immortalized cell line” refer to cells that are passaged or modified to proliferate indefinitely and evade normal cellular senescence.

As used herein, the term “population doubling level (PDL)” refers to the total number of times the cells in the population have doubled since their primary isolation in vitro or since their time of procurement. Mathematically this is described as PDLf=PDLi+PD, where PD=(LN(Yf/Yi)/LN(2)); PDLi=previously calculated PDL; PDLf=final calculated PDL; Yi=initial population of cells; and Yf=final population of cells.

As used herein the term “passaged cell number” refers to the number of times the cells in the culture have been subcultured. This may occur without consideration of the inoculation densities or recoveries involved.

As used herein, the term “differentiation capacity” refers to a cells ability to differentiate to a particular cell lineage, stem cell, progenitor cell, or terminally differentiated cell.

As used herein, the term “fragment” or “portion” when referring a protein or a polynucleotide refers to a protein that comprises a domain, portion, or fragment of a parent or reference protein or polypeptide. The term “portion” can be used interchangeably with the term “functional portion.” The term “fragment” can be used interchangeably with the term “functional fragment.” The terms “functional portion” or “functional fragment” retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent protein or polypeptide, or provides a biological benefit. A “functional portion” or “functional fragment” of a protein or polypeptide has “similar binding” or “similar activity” when the functional portion or fragment displays no more than a 50% reduction in performance in a selected assay as compared to the parent or reference protein or polypeptide (preferably no more than 20% or 10%, or no more than a log difference as compared to the parent or reference with regard to affinity).

As used herein, the term “cultivation infrastructure” refers to the environment in which isolated bovine cells, including myogenic or fibroblastic bovine cells and bovine cells generated by the methods described herein, are cultured (i.e., the environment in which the bovine cell is cultivated).

As used herein, the term “replicative capacity” means the number of doubling times that a cell or population of cells is capable starting from primary isolation or procurement. The population doubling level or PDL is a measurement of replicative capacity.

As used herein, the term “substantially free of” or “substantially free from” means the amount (e.g., absolute number within a population or concentration/percentage within a population) of a cell or cell type is below a value where the cell or cell type, or any cell derived therefrom, could contribute significantly to the population. For example, a population substantially free of a cell means that upon differentiation of the population the cell does not contribute progeny to the differentiated population upon culture, e.g., the cell and its progeny are diluted out to be undetectable as the cell culture doubles one, two, three, or four times.

As used herein, the terms “transformed,” “transduced,” and “transfected” are used interchangeably unless otherwise noted. Each term refers to introduction of a nucleic acid sequence or polypeptide into a cell (e.g., an immortalized cell).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

### 4.2. Method for Extending the Replicative Capacity of a Bovine Cell

Provided herein is a method for extending the replicative capacity of a bovine cell or population of bovine cells, including bovine cells which are not immortalized, the method comprising: (a) isolating a population of cells from bovine tissue or from a source of bovine cells, including a stored bovine cell culture or master cell bank; (b) engineering the bovine cells to express or increase expression of telomere reverse transcriptase (TERT) and a first protein that is capable of modulating cell proliferation; and (c) culturing the population of bovine cells in a cultivation infrastructure. In embodiments, provided is a method for extending the replicative capacity of a bovine cell or population of bovine cells, the method comprising (a) isolating a population of cells from bovine tissue; (b) introducing into a cell of the population of cells a polynucleotide encoding telomere reverse transcriptase (TERT); (c) introducing into a cell of the population of cells, including into the cell of step (b), at least a first polynucleotide encoding a first protein that is capable of modulating cell proliferation; wherein both the polynucleotide encoding TERT and the polynucleotide encoding the first protein that is capable of modulating cell proliferation are operably linked to one or more regulatory elements that promote (either constitutively or inducibly) expression in the bovine cells; and (d) culturing the population of bovine cells in a cultivation infrastructure. In embodiments, one or both of the polynucleotides encoding TERT and/or the protein capable of modulating cell proliferation are transiently transfected into the bovine cells such that the polynucleotides do not become integrated into the genome of the bovine cells. In embodiments, bovine cells having the polynucleotide encoding TERT and the polynucleotide encoding the protein capable of modulating cell proliferation are selected by selectable markers-such as antibiotic resistance or detectable markers, such as fluorescent markers, associated with the polynucleotides. In embodiments, the polynucleotide encoding TERT and the polynucleotide encoding the protein capable of modulating cell proliferation are on the same contiguous polynucleotide.

In some embodiments, wherein, after expressing the polynucleotide encoding TERT and the first polynucleotide encoding the first protein that is capable of modulating cell proliferation, the population of bovine cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more). In some embodiments, wherein, after expressing the polynucleotide encoding TERT and the first polynucleotide encoding the first protein that is capable of modulating cell proliferation, the population of bovine cells have a reduced doubling time (e.g. 1 doubling every 12 hours, every 13 hours, every 14 hours, every 15 hours, every 16 hours, every 17 hours or every 18 hours) compared to the doubling time (e.g., 1 double every 20 hours or more) of a bovine cell that has not been engineered to express or have increased expression of TERT and the at least one protein that is capable of modulating cell proliferation. In embodiments, the population of bovine cells that has been produced by methods described herein retain replicative capacity even after 50, 60, 70, 80, 90 or 100 doublings, e.g., can be subcultured with a PDL of at least 50, 60, 70, 80, 90, or 100, and maintain a doubling time of 20 hours or less. Introducing the polynucleotides by transient transfection and not integration into the genome provides the benefit that the introduced nucleic acids are not detectable after 5, 10, 15 or 20 doublings and, in addition, the backbone or non-coding portion of the vector or construct used to introduce the polynucleotides does not need to be excised from the bovine cells.

Provided herein is a method for extending the replicative capacity of a bovine cell, comprising: (a) isolating a population of cells from bovine tissue; (b) incorporating into the genome of a cell of the population of cells a polynucleotide encoding telomere reverse transcriptase (TERT); (c) introducing into the cell of step (b) at least a first polynucleotide encoding a first protein that is capable of modulating cell proliferation; wherein both the polynucleotide encoding TERT and the polynucleotide encoding the first protein that is capable of modulating cell proliferation are operably linked to one or more regulatory elements that promote (either constitutively or inducibly) expression in the bovine cells; and (d) culturing the monoculture of bovine cells in a cultivation infrastructure. In some embodiments, wherein, after incorporating into the genome the polynucleotide encoding TERT and introducing into the cell the first polynucleotide encoding the first protein that is capable of modulating cell proliferation, the population of bovine cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more). In some embodiments, wherein, after incorporating into the genome the polynucleotide encoding TERT and introducing into the cell the first polynucleotide encoding the first protein that is capable of modulating cell proliferation, the population of bovine cells have a reduced doubling time (e.g. 1 doubling every 12 hours, every 13 hours, every 14 hours, every 15 hours, every 16 hours, every 17 hours or every 18 hours) compared to the doubling time (e.g., 1 double every 20 hours) of a bovine cell that has not been engineered to express or have increased expression of TERT and the first polynucleotide encoding the first protein that is capable of modulating cell proliferation.

Provided herein is a method for extending the replicative capacity of a bovine cell, comprising: (a) isolating a population of cells from bovine tissue; (b) introducing into a cell of the population of cells a polynucleotide encoding telomere reverse transcriptase (TERT); (c) incorporating into the genome of the cell of step (b) at least a first polynucleotide encoding a first protein that is capable of modulating cell proliferation; wherein both the polynucleotide encoding TERT and the polynucleotide encoding the first protein that is capable of modulating cell proliferation are operably linked to one or more regulatory elements that promote (either constitutively or inducibly) expression in the bovine cells; and (d) culturing the monoculture of bovine cells in a cultivation infrastructure. In some embodiments, wherein, after introducing into the cell the polynucleotide encoding TERT and incorporating into the genome the at least first polynucleotide encoding the first protein that is capable of modulating cell proliferation, the population of bovine cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more). In some embodiments, wherein, after introducing into the cell the polynucleotide encoding TERT and incorporating into the genome the first polynucleotide encoding the first protein that is capable of modulating cell proliferation, the population of bovine cells have a reduced doubling time (e.g. 1 doubling every 12 hours, every 13 hours, every 14 hours, every 15 hours, every 16 hours, every 17 hours or every 18 hours) compared to the doubling time (e.g., 1 double every 20 hours) of a bovine cell that has not been engineered to express or have increased expression of TERT and the first polynucleotide encoding a protein that is capable of modulating cell proliferation.

Provided herein is a method for extending the replicative capacity of a bovine cell, comprising: (a) isolating a population of cells from bovine tissue; (b) incorporating into the genome of a cell of the population of cells a polynucleotide encoding telomere reverse transcriptase (TERT); (c) incorporating into the genome the cell of step (b), at least a first polynucleotide encoding a first protein that is capable of modulating cell proliferation; wherein both the polynucleotide encoding TERT and the polynucleotide encoding the first protein that is capable of modulating cell proliferation are operably linked to one or more regulatory elements that promote (either constitutively or inducibly) expression in the bovine cells and (d) culturing the monoculture of bovine cells in a cultivation infrastructure. In some embodiments, wherein, after incorporating into the genome the polynucleotide encoding TERT and incorporating into the genome the first polynucleotide encoding the first protein that is capable of modulating cell proliferation, the population of bovine cells have a population doubling level (PDL) of at least 55 (e.g., at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more). In some embodiments, wherein, after incorporating into the genome the polynucleotide encoding TERT and incorporating into the genome the first polynucleotide encoding the first protein that is capable of modulating cell proliferation, the population of bovine cells have a reduced doubling time (e.g. 1 doubling every 12 hours, every 13 hours, every 14 hours, every 15 hours, every 16 hours, every 17 hours or every 18 hours) compared to the doubling time (e.g., 1 double every 20 hours) of a bovine cell that has not been engineered to express or have increased expression of TERT and the first polynucleotide encoding a protein that is capable of modulating cell proliferation.

In some embodiments, the population of bovine cells comprising the bovine cell engineered to overexpress the polynucleotide encoding TERT and the first polynucleotide encoding the first protein that is capable of modulating cell proliferation has a population doubling level (PDL) at least 1.1-fold (e.g., 1.2-fold, 1.3-fold, 1.4-fold, 1.5-fold, 1.6-fold, 1.7-fold, 1.8-fold, 1.9-fold, 2.0-fold, 2.1-fold, 2.2-fold, 2.3-fold, 2.4-fold, 2.5-fold, 2.6-fold, 2.7-fold, 2.8-fold, 2.9-fold, or 3-fold) greater than a bovine cell that has not been engineered to express or have increased expression of TERT and the at least one polynucleotide encoding the first protein that is capable of modulating cell proliferation. In some embodiments, the population of bovine cells engineered to overexpress the polynucleotide encoding TERT and the first polynucleotide encoding the first protein that is capable of modulating cell proliferation has a population doubling level (PDL) at least 1.5-fold greater than a bovine cell that has not been engineered to express or have increased expression of TERT and the protein capable of modulating cell proliferation. In some embodiments, the population of bovine cells comprising the polynucleotide encoding TERT and the first polynucleotide encoding the first protein that is capable of modulating cell proliferation has a population doubling level (PDL) at least 2-fold greater than a bovine cell that has not been engineered to express or have increased expression of TERT and the protein capable of modulating cell proliferation.

In some embodiments, the method includes integrating at least a first integrase recognition site prior to step (b) or step (c). In some embodiments, the method includes integrating two, three, four, five, six, seven, eight, nine, or ten, or more integrase recognition sites. In some embodiments, the integrase recognition sequences is a cognate sequence to an integrase recognition sequence on a polynucleotide (e.g., a polynucleotide comprising the polynucleotide encoding TERT or a polynucleotide comprising the polynucleotide encoding the protein that is capable of modulating cell proliferation). For example, in the presence of an integrase (e.g., PhiC31) the cognate pair of integrase recognition sites enable integration of a polynucleotide into the genome of the cell (e.g., the polynucleotide encoding TERT or the polynucleotide encoding the protein that is capable of modulating cell proliferation).

In some embodiments, incorporating into the genome the polynucleotide encoding TERT (as a donor polynucleotide) is effected by introducing into the cell: (i) the polynucleotide encoding TERT and an integrase recognition sequence; and (ii) an integrase or a polynucleotide encoding an integrase expressible in the bovine cell, whereby the donor polynucleotide is incorporated into the genome by the integrase at the first integrase recognition site integrated into the genome.

In some embodiments, incorporating into the genome the at least first polynucleotide encoding a protein that is capable of modulating cell proliferation is effected by introducing into the cell: (i) the first polynucleotide encoding a first protein that is capable of modulating cell proliferation; and (ii) an integrase or a polynucleotide encoding an integrase, whereby the first polynucleotide is incorporated into the genome by the integrase at the first integrase recognition site integrated into the genome.

In some embodiments, incorporating into the genome the polynucleotide encoding TERT is effected by a method selected from: viral vector-mediated integration, non-viral vector mediated integration, CRISPR/Cas9-mediated integration, zinc-finger nuclease-mediated integration, and TALEN-mediate integration.

In some embodiments, incorporating into the at least first polynucleotide encoding a first protein that is capable of modulating cell proliferation is effected by a method selected from: viral vector-mediated integration, non-viral vector mediated integration, CRISPR/Cas9-mediated integration, zinc-finger nuclease-mediated integration, and TALEN-mediate integration.

In some embodiments, introducing into the cell the polynucleotide encoding TERT, the first polynucleotide encoding a first protein capable of modulating cell proliferation, or both, is effected such that the introduced polynucleotide(s) do not integrate into the genome of the cell.

In some embodiments, wherein steps of introducing (or incorporating into the genome) the polynucleotide encoding TERT and introducing (or incorporating into the genome) the polynucleotide encoding the protein that is capable of modulating cell proliferation are performed concurrently.

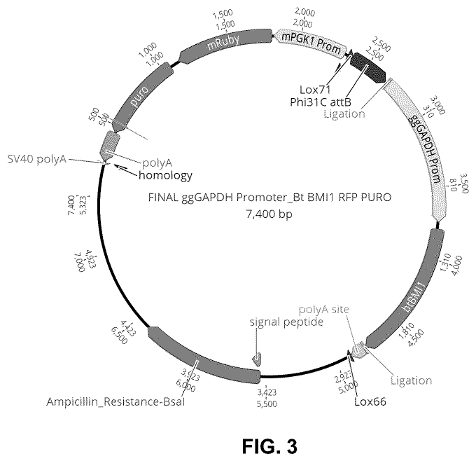

A non-limiting workflow of the methods described herein is as shown in FIG. 2.

### 4.3. Method for Improving Differentiation Capacity

A method for improving differentiation capacity of a bovine cell line, which is not immortal, comprising: (a) isolating a population of cells from bovine skin or muscle or a source of non-immortal bovine cells; (b) introducing into a cell of the population of cells a polynucleotide encoding telomere reverse transcriptase (TERT) under control of one or more regulatory elements that promote expression in the bovine cell; (c) introducing into the cell of step (b) at least a first polynucleotide encoding a protein that is capable of modulating cell proliferation (or vice versa where the polynucleotide encoding TERT is introduced into cells containing the polynucleotide encoding the protein capable of cell proliferation; or simultaneously) under control of one or more regulatory elements that promote expression in the bovine cell; (d) culturing the monoculture of bovine cell in a cultivation infrastructure; and (e) inducing myogenic-specific differentiation.

### 4.4. Method of Increasing Cell Density

Provided herein are methods of increasing the cell density of a culture including the bovine cell line, including a bovine cell line which is not immortalized, where the method includes introducing into, or incorporating into the genome of, a cell of the population of cells: a polynucleotide encoding telomere reverse transcriptase (TERT); and introducing into, or incorporating into the genome of, the cell containing the TERT polynucleotide at least a first polynucleotide encoding a first protein that is capable of modulating cell proliferation or vice versa, and both of which polynucleotides are expressed in the bovine cell; and culturing the bovine cells in a cultivation infrastructure, thereby increasing the cell density of the culture as compared to controls. In embodiments, the TERT and protein capable of modulating cell proliferation are expressed transiently in the first or first 5 or first 10 cell doublings thereby increasing the cell density of the bovine cell culture as it continues to grow.

In some embodiments, an increase in the cell density of a culture (e.g., suspension culture) using the methods described herein is about 1.025 fold, 1.05 fold, 1.10-fold, 1.15-fold, 1.20-fold, 1.25-fold, 1.30 fold, 1.35-fold, 1.40-fold, 1.45-fold, 1.5-fold, 2-fold, 2.5-fold, 3-fold, 3.5-fold, 4-fold, 4.5-fold, 5-fold, 7.5-fold, 10-fold, 15-fold, 20-fold, 25-fold, 30-fold, 40-fold, or even about 50-fold, 75-fold, 100-fold, 150-fold, or about 200-fold, compared to the density of a culture comprising bovine cells that do not overexpress a telomere reverse transcriptase (TERT) and a first protein that is capable of modulating cell proliferation.

In some embodiments, an increase in the density of cells in a culture (e.g., suspension culture) using the methods described herein is at least 10%, at least 15%, at least 20%, at least 25%, at least 30%, at least 35%), at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, at least 1000%, compared to the density of a culture comprising bovine cells that do not overexpress telomere reverse transcriptase (TERT) and a first protein that is capable of modulating cell proliferation.

In some embodiments, methods described herein increase the density of cells in a culture of bovine cells by increasing the rate of proliferation of cells in the culture. In some embodiments, the increase in the rate of cell proliferation is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%), including values and ranges therebetween, compared to the density of a culture comprising bovine cells that do not overexpress telomere reverse transcriptase (TERT) and a first protein that is capable of modulating cell proliferation In some embodiments, the increase in the rate of cell proliferation is about 25-1000%, about 25-750%, about 25-500%, about 50-1000%, about 50-750%, about 50-500%, about 100-1000%, about 100-750%, or about 100-500%, including values and ranges therebetween, compared to the density of a culture comprising bovine cells that do not overexpress telomere reverse transcriptase (TERT) and a first protein that is capable of modulating cell proliferation.

In some embodiments, methods described herein increase the cell density of a culture of bovine cells by decreasing cell death within the population of cells. In some embodiments, the decrease in cell death is at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, including values and ranges therebetween, compared to the density of a comprising bovine cells that do not overexpress telomere reverse transcriptase (TERT) and a first protein that is capable of modulating cell proliferation. In some embodiments, a decrease in the rate of cell death within the population of bovine cells is about 2.5-10%, about 2.5-75%, about 2.5-50%, about 5.0-100%, about 5.0-75%, about 5.0-50%, about 10-100%, about 10-75%, or about 10-50%, including values and ranges therebetween, compared to the density of a culture comprising bovine cells that do not overexpress telomere reverse transcriptase (TERT) and a first protein that is capable of modulating cell proliferation.

In some embodiments, using the methods described herein, the density of cells in a culture may reach about 1E4 cells/mL, about 1E5 cells/mL, about 1E6 cells/mL, about 1E7 cells/mL, about 1E8 cells/mL, about 1E9 cells/mL, about 1E10 cells/mL, about 1E11 cells/mL, about 1E12 cells/mL, or about 1E13 cells/mL (cells in suspension culture or cells in the cellular biomass/mL of cultivation infrastructure), including values and ranges therebetween.

In some embodiments, using the methods described herein, the density of cells in a culture (e.g., suspension culture) may reach about 1 g/L, 5 g/L, 10 g/L, 25 g/L, 50 g/L, 75 g/L, 100 g/L, 150 g/L, 200 g/L, 250 g/L, 300 g/L, 350 g/L, 400 g/L, 450 g/L, 500 g/L, 550 g/L, 600 g/L, 650 g/L, 700 g/L, 750 g/L, 800 g/L, 850 g/L, 900 g/L, or 1000 g/L (g of cellular biomass/L of cultivation infrastructure), including values and ranges therebetween. In some embodiments, the density of cells in a culture (e.g., suspension culture) may range from about 1 g/L to about 5 g/L, about 1 g/L to about 750 g/L, about 1 g/L to about 500 g/L, about 1 g/L to about 250 g/L, about 1 g/L to about 100 g/L, about 1 g/L to about 50 g/L, about 5 g/L to about 1000 g/L, about 5 g/L to about 750 g/L, about 5 g/L to about 500 g/L, about 5 g/L to about 250 g/L, about 5 g/L to about 100 g/L, about 5 g/L to about 50 g/L, about 25 g/L to about 1000 g/L, about 25 g/L to about 750 g/L, about 25 g/L to about 500 g/L, about 25 g/L to about 300 g/L, about 25 g/L to about 250 g/L, about 25 g/L to about 100 g/L, about 50 g/L to about 1000 g/L, about 50 g/L to about 750 g/L, about 50 g/L to about 500 g/L, about 50 g/L to about 300 g/L, about 50 g/L to about 250 g/L, about 100 g/L to 1000 g/L, about 100 g/L to about 750 g/L, about 100 g/L to about 500 g/L, about 200 g/L to about 1000 g/L, about 200 g/L to about 750 g/L, about 200 g/L to about 500 g/L, about 300 g/L to about 1000 g/L, about 300 g/L to about 800 g/L, about 400 g/L to about 1000 g/L, or about 500 g/L to about 1000 g/L including values and ranges therebetween.

### 4.5. Methods for Producing Cell-Based Meat Suitable for Consumption

Provided herein are in vitro methods for producing cell-based meat suitable for consumption, comprising: introducing into (including transfecting, transiently transfecting, or stably incorporating into the genome of) a cell of the population of cells a polynucleotide encoding telomere reverse transcriptase (TERT) under control of one or more regulatory elements that promote expression in the cells; introducing into (including transfecting, transiently transfecting, or stably incorporating into the genome of) the cell of step (b) at least a first polynucleotide encoding a first protein that is capable of modulating cell proliferation under control of one or more regulatory elements that promote expression in the cells; inducing myogenic specific differentiation, wherein the differentiated cells form myotubes and multinucleated myotubes; culturing the myocytes and myotubes to generate skeletal muscle fibers, thereby producing a cell-based meat suitable for consumption. In embodiments the cells are bovine cells. In some embodiments, the in vitro method for producing cell-based meat suitable for consumption includes a step of adapting the cells to grow in suspension. In some embodiments, the in vitro method for producing cell-based meat suitable for consumption includes a step of culturing the cells in a cultivation infrastructure. In some embodiments, provided herein is cell-based meat suitable for consumption produced by the in vitro methods. In embodiments, the bovine cells, after introduction of the polynucleotide encoding TERT and the polynucleotide encoding the protein capable of modulating cell production, are cultured to a target cell density, including in suspension culture, for example 6E6 to 10E6, and then used to seed a substrate for adherent culture under conditions that promote myogenic differentiation. Alternatively, the cell-based meat product is produced from the cells cultivated in the cultivation infrastructure, including by suspension culture, concentrating and/or removing moisture from the cell biomass and processing the cell biomass into a cell-based meat product.

In some embodiments, the in vitro method for producing cell-based meat suitable for consumption includes introducing into the cell one or more agents (e.g., an effector protein) that reduces or eliminates expression of TERT and/or the at least a first protein capable of modulating cell proliferation. In some embodiments, the in vitro method for producing cell-based meat suitable for consumption includes introducing into the cell one or more agents (e.g., an effector protein) that disrupts expression, at the site of genome integration, of the polynucleotide encoding TERT. In some embodiments, the in vitro method for producing cell-based meat suitable for consumption includes introducing into the cell one or more agents (e.g., an effector protein) that disrupts expression, at the site of genome integration, of the polynucleotide encoding the at least a first protein capable of modulating cell proliferation. In embodiments where the bovine cells were transiently transfected with the polynucleotides that encode TERT and the protein that is capable of modulating cell proliferation, the bovine cells that are used to produce the cell-based meat for consumption, including those used for adherent cell culture and differentiation to myogenic cells, do not contain detectable polynucleotides encoding TERT or the protein capable of modulating cell proliferation, or nucleic acid associated therewith, including the regulatory elements or backbone of the construct or vector used to transfect the cells.

In some embodiments, the in vitro method for producing cell-based meat suitable for consumption includes introducing into the cell an agent (e.g., an effector protein) that induces a genetic perturbation in the integrated polynucleotide (e.g., the integrated polynucleotide encoding TERT or the integrated polynucleotide encoding the protein that is capable of modulating cell proliferation). In some embodiments, the agent is an effector selected from a nuclease, a recombinase, and an integrase.

In some embodiments, the in vitro method for producing cell-based meat suitable for consumption includes introducing into the cell a recombinase. In some embodiments, the recombinase is a CRE recombinase. In certain embodiments using methods described herein, there is recombinase or integrase utilized or present in cells because the polynucleotides encoding TERT and the protein capable of modulating cell proliferation are transiently transfected into the bovine cells.

Non-limiting examples of myogenic differentiation are as described in WO2019014652A1, WO2018208628A1, and WO2015066377A1, each of which is herein incorporated by reference in their entireties.

In some embodiments, the skeletal muscle produced according to the methods described herein can be processed as a raw, uncooked food product (cultured meat) or as a cooked food product or as a cooked/uncooked food ingredient. In some embodiments, processing comprises withdrawal of the culture medium that supports the viability, survival, growth or expansion (e.g., increase in total protein content of the non-naturally occurring myogenic cells) and differentiation of the myogenic cells. Withdrawal may comprise physical removal of the culture medium or altering the composition of the culture medium, for example, by addition of components that would reduce or prevent further expansion and/or differentiation of the cell line or cells-derived from the cell line or by depletion of components that support expansion and/or differentiation of the cell line or cells derived from the cell line.

### 4.6. Telomere Reverse Transcriptase (TERT)

In some embodiments, the method provided herein includes introducing into a polynucleotide encoding telomerase reverse transcriptase (TERT). As used herein, “TERT” refers to telomerase reverse transcriptase (TERT) gene or TERT polypeptide that is a ribonucleoprotein polymerase that maintains telomere ends by addition of the telomere repeat TTAGGG. Telomerase expression plays a role in cellular senescence, as it is normally repressed in postnatal somatic cells resulting in progressive shortening of telomeres. In some embodiments, cells ectopically express the TERT polynucleotide. In some embodiments, the cells are genetically modified and carry stable integrations of one or more copies of the TERT polynucleotide. Exemplary methods for immortalizing a cell line are as described in WO2019014652A1, which is herein incorporated by reference in its entirety.

In some embodiments, increased expression of TERT may be achieved using different approaches. In some embodiments, increased expression of TERT may be achieved by ectopically expressing TERT, including under the control of a regulatory element such as a promoter which promotes expression of TERT in the bovine cell, such as GAPDH (e.g., SEQ ID NO: 17), or an inducible promoter. In some embodiments, increased expression of TERT may be achieved by introducing targeted mutations in the TERT promoter. In some embodiments, increased expression of TERT may be achieved by activating endogenous TERT expression by an engineered transcriptional activator. In some embodiments, increased expression of TERT may be achieved by transiently transfecting TERT mRNA.

The polynucleotide encoding TERT can be from any organism. The TERT polynucleotide can be from bacteria, plants, fungi, and archaea. The TERT polynucleotide can be from any animal, such as vertebrate and invertebrate animal species. The TERT polynucleotide can be from any vertebrate animal species such as mammals, reptiles, birds, amphibians, and the like. The TERT polynucleotide can be from any mammalian species, such as a human, murine, bovine, porcine, and the like. The TERT is preferably from the same species as the cells, for example, bovine TERT may be expressed in bovine cells. The TERT may have an amino acid sequence at least 80%, 85%, 90%, 95%, or 99% or 100% identical to SEQ ID NO: 1, and may be encoded by a nucleotide sequence which is SEQ ID NO: 2 or at least at least 80%, 85%, 90%, 95%, or 99% or 100% identical to SEQ ID NO: 2.

Introducing into a bovine cell a TERT protein alone is not sufficient to expand the replicative capacity of the cell. In such embodiments, one or more additional proteins that are capable of modulating cell proliferation can be introduced into the cell to achieve expansion of the replicative capacity of the cell. For example, a polynucleotide comprising the coding sequence of a BMI-1 protein, a CDK4 protein, a Cyclin D1 protein, a PCG1α protein, a DKC1 protein, a YAP1 protein, or a Nanog protein, or a combination thereof, can be introduced into the cell line and expressed in the cell line to help immortalize or expand the replicative capacity of the cell.

### 4.7. Genes Capable of Modulating Cell Proliferation

Provided herein are methods that include introducing into or incorporating into the genome of a cell a polynucleotide encoding telomere reverse transcriptase (TERT) and at least a first polynucleotide encoding a first protein that is capable of modulating cell proliferation. In some embodiments, the protein that is capable of modulating cell proliferation is BMI-1, CDK4, Cyclin D1, PCG1α, Nanog, DKC1 or YAP.

### 4.7.1. Polycomb Complex Protein or Polycomb Ring Finger (BMI-1)

In some embodiments, the methods provided herein include introducing into a cell a polynucleotide comprising a coding sequence of polycomb complex protein or polycomb ring finger (BMI-1) or a fragment thereof. As used herein, “BMI-1” or “BMI1” refers to the polycomb complex protein or polycomb ring finger that is involved in mediating growth and development. Without wishing to be bound by theory, BMI-1 prevents senescence and immortalizes cells via telomerase activation. In some cases, elevation of BMI-1 expression is closely correlated to the increased telomerase activity. BMI-1 aids in normal embryonic development and stem cell maintenance and is highly expressed in embryonic stem cells. Additionally, BMI-1 regulates the tumor suppressor proteins p16Ink4a and p14Arf, can promote CDK4 and CDK6 activity, directly regulates p53 stability, and maintains mitochondrial function and redox homeostasis. BMI-1 also is a key component in DNA Damage Response (DDR), for example, BMI-1 recruits the DDR machinery to DNA double-strand breaks (DSBs).

In some embodiments, the cells are modified to overexpress the coding sequence of a BMI-1 protein. In some embodiments, the cells are genetically modified and carry stable integrations of one or more copies of a BMI-1 coding sequence. In some embodiments, the cells overexpress the coding sequence of BMI-1 protein at levels sufficient to induce cell proliferation and/or immortalize the cell.

In some embodiments, the BMI-1 coding sequence is selected from any metazoan species. In some embodiments, the BMI-1 coding sequence is from any animal, such as vertebrate and invertebrate animal species. In some embodiments, the BMI-1 coding sequence is from any vertebrate animal species such as mammals, reptiles, birds, amphibians, and the like. In some embodiments, the BMI-1 coding sequence is from any mammalian species such as a human, murine, bovine, porcine, poultry, and the like. In some embodiments, the coding sequence of the BMI-1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of BMI-1 protein is derived from Bos taurus.

In some embodiments, increasing expression of BMI-1 may be achieved using different approaches. In some embodiments, the expression is inducible. In some embodiments, the method comprises expressing polynucleotides comprising the coding sequence of BMI-1. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the constructs are not integrated into the cell's genome. In some embodiments, the construct is episomal. In some embodiments, the expression of the BMI-1 gene involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding BMI-1 is integrated into the cell's genome but undergoes silencing.

In some embodiments, a polynucleotide comprising a coding sequence of BMI-1 may encode any homolog of BMI-1, including BMI-1 paralogs, or any other BMI-1 paralogs, or an BMI-1 protein translated from any splice variants of an BMI-1 gene, or may comprise any mutations in the BMI-1 gene sequence including, but not limited to nucleotide deletions, truncations, fusions, or substitutions. Mutations may be synthetic or naturally occurring.

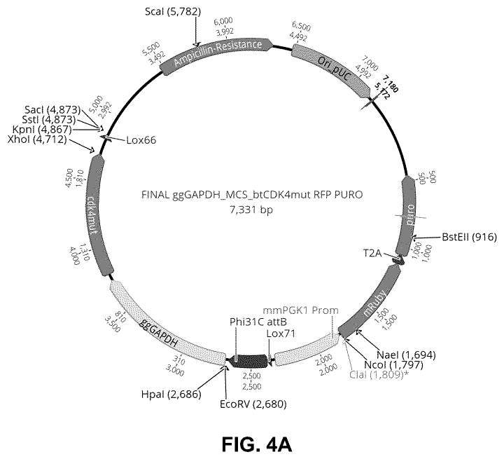

In some embodiments, BMI-1 refers to the BMI-1 gene or BMI-1 protein, or fragment or variant thereof (e.g., a BMI-1 protein having one or more (e.g., one, two, three, four, five, six, seven, eight, nine, ten, or more amino acid substitutions, deletions or insertions as compared to a wild type BMI-1 protein)). FIG. 3 shows a plasmid map of a construct used to introduce the coding sequence of BMI into any of the cells described herein.

In some embodiments, an BMI-1 protein comprises an amino acid sequence having at least 80% (e.g., at least 85%, 90, 95%, 96%, 97%, 98%, or 99%) sequence identity to the amino acid sequence of SEQ ID NO: 3. In some embodiments, the BMI-1 protein sequence comprises an amino acid sequence of SEQ ID NO: 3.

In some embodiments, a polynucleotide encoding BMI-1 comprises a nucleic acid sequence having at least 80% (e.g., at least 85%, 90, 95%, 96%, 97%, 98%, or 99%) sequence identity to the nucleic acid sequence of SEQ ID NO: 4. In some embodiments, the polynucleotide encoding BMI-1 comprises a nucleic acid sequence of SEQ ID NO: 4.

In some embodiments, introducing the polynucleotide comprising the coding sequence of the BMI-1 protein alone is not sufficient to immortalize the cell. In such embodiments, one or more additional proteins that are capable of modulating cell proliferation can be introduced into the cell. For example, a polynucleotide comprising the coding sequence of a TERT protein, a CDK4 protein, a Cyclin D1 protein, a PCG1α protein, a DKC1 protein, a YAP1 protein, or a Nanog protein, or a combination thereof, can be introduced into the cell line to help immortalize or expand the replicative capacity of the cell.

### 4.7.2. Cyclin-Dependent Kinase 4 (CDK4)

In some embodiments, the methods provided herein include introducing into a cell a polynucleotide comprising a coding sequence of cyclin-dependent kinase 4 (CDK4) or a fragment thereof. Cyclin-dependent kinases are serine/threonine kinases whose activity depends on a regulatory subunit, for example, a cyclin (e.g., cyclin D1). The cyclin-dependent kinase family consists of CDK-1, CDK-4, and CDK-5, Cyclin-dependent kinases (CDKs). In some embodiments, the cyclin-dependent kinase is CDK4. In some embodiments, the cyclin-dependent kinase is a CKD1. In some embodiments, the cyclin-dependent kinase is a CDK5. As used herein, “CDK4” refers to the cyclin-dependent kinase (Cdk4) gene or CDK4 protein.

CDK4 phosphorylates cell cycle related proteins to promote cell cycle progression and regulates transcription of other genes to mediate cell proliferation. CDK4 is also involved in DNA damage repair and cancer cell survival.

In some embodiments, the cells are modified to overexpress the coding sequence of a cyclin-dependent kinase (e.g., CKD4, CDK1, and CDK5) protein. In some embodiments, the cells are genetically modified and carry stable integrations of one or more copies of a cyclin-dependent kinase coding sequence (e.g., one or more copies of a cyclin-dependent kinase coding sequence). In some embodiments, the cells overexpress the coding sequence of a cyclin-dependent kinase protein (e.g., CDK4, CDK1, and CDK5) at levels sufficient to increase cell proliferation and/or immortalize the cell.

In some embodiments, the cyclin-dependent kinase coding sequence is selected from any metazoan species. In some embodiments, the cyclin-dependent kinase coding sequence is from any animal, such as vertebrate and invertebrate animal species. In some embodiments, the cyclin-dependent kinase coding sequence is from any vertebrate animal species such as mammals, reptiles, birds, amphibians, and the like. In some embodiments, the cyclin-dependent kinase coding sequence is from any mammalian species such as a human, murine, bovine, porcine, poultry, and the like. In some embodiments, the coding sequence of the cyclin-dependent kinase protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the cyclin-dependent kinase protein is derived from Bos taurus.

In some embodiments, increasing expression of a cyclin-dependent kinase may be achieved using different approaches. In some embodiments, the expression is inducible. In some embodiments, the method comprises expressing polynucleotides comprising the coding sequence of a cyclin-dependent kinase (e.g., a CDK4, CDK1, and CDK5). In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the cyclin-dependent kinase gene (e.g., CDK4 gene, CDK1 gene, and CDK5 gene) involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding the cyclin-dependent kinase is integrated into the cell's genome but undergoes silencing.

In the methods described herein, a polynucleotide comprising a coding sequence of a cyclin-dependent kinase (e.g., a coding sequence of CDK4, a coding sequence of CDK1, and a coding sequence of CKD5). In some embodiments, a cyclin-dependent kinase may encode any homolog of a cyclin-dependent kinase, including cyclin-dependent kinase paralogs, or a cyclin-dependent kinase protein translated from any splice variants of a cyclin-dependent kinase gene, or may comprise any mutations in the cyclin-dependent kinase gene sequence including, but not limited to nucleotide deletions, truncations, fusions, or substitutions. Mutations may be synthetic or naturally occurring.

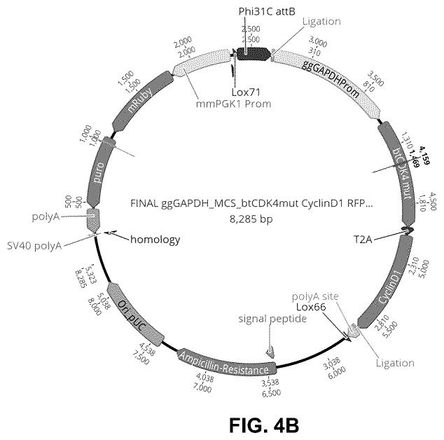

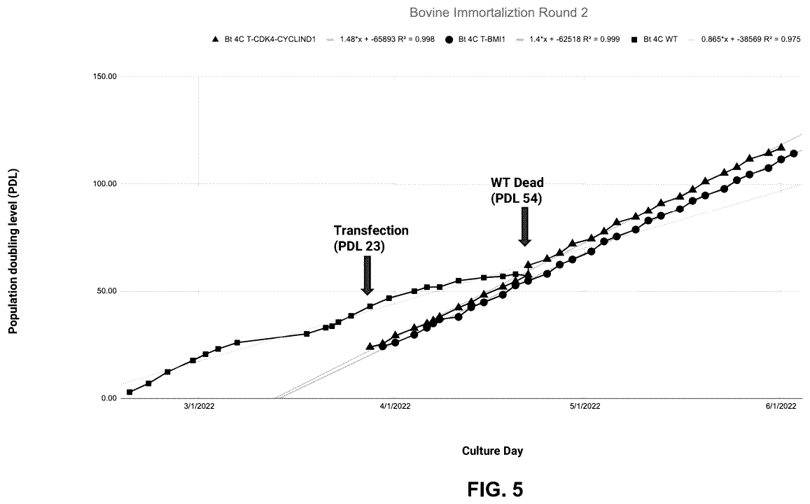

In some embodiments, a cyclin-dependent kinase protein (e.g., a CDK4 protein, a CDK1 protein, and a CDK6 protein) refers to the Cdk4 gene, Cdk1 gene, and Cdk5 gene or CDK4 protein, CDK1 protein, or a CDK5 protein, respectively, or a fragment or variant thereof (e.g., a cyclin-dependent kinase protein having one or more (e.g., one, two, three, four, five, six, seven, eight, nine, ten, or more amino acid substitutions, deletions or insertions as compared to a wild type cyclin-dependent kinase protein)). FIG. 4A or 4B shows a plasmid map of a construct used to introduce the coding sequence of the cyclin-dependent kinase (e.g., the CDK4, CKD1, CDK5) protein into any of the cells described herein.

In some embodiments, a CDK4 protein comprises an amino acid sequence having at least 80% ((e.g., at least 85%, 90, 95%, 96%, 97%, 98%, or 99%) sequence identity to the amino acid sequence of SEQ ID NO: 5. In some embodiments, the CDK4 protein sequence comprises the amino acid sequence of SEQ ID NO: 5.

In some embodiments, a polynucleotide encoding CDK4 comprises a nucleic acid sequence having at least 80% (e.g., at least 85%, 90, 95%, 96%, 97%, 98%, or 99%) sequence identity to the nucleic acid sequence of SEQ ID NO: 6. In some embodiments, the polynucleotide encoding CDK4 comprises a nucleic acid sequence of SEQ ID NO: 6.

In some embodiments, introducing the polynucleotide comprising the coding sequence of the cyclin-dependent kinase protein (e.g., CDK4 protein, CDK1 protein, and a CDK5 protein) alone is not sufficient to increase cell proliferation and/or immortalize the cell. In such embodiments, one or more additional proteins that are capable of modulating cell proliferation can be introduced into the cell. For example, a polynucleotide comprising the coding sequence of a TERT protein, a BMI-1 protein, a Cyclin D1 protein, a PCG1α protein, a DKC1 protein, a YAP1 protein, or a Nanog protein, or a combination thereof, can be introduced into the cell line to help immortalize or expand the replicative capacity of the cell.

### 4.7.3. Cyclin D1

In some embodiments, the methods provided herein include introducing into a cell a polynucleotide comprising a coding sequence of cyclin D1 (CCND1) or a fragment thereof. As used herein, “cyclin D1” or “CYCLIND1” or “CCND1” refers to the cyclin D1 (Ccnd1) gene or CCND1/cyclin D1 protein. Without wishing to be bound by theory, cyclin D1 proto-oncogene is an important regulator of G1 to S phase progression in many different cell types. Cyclin D1, together with its binding partners cyclin dependent kinase 4 and 6 (CDK4 and CDK6), it forms active complexes that promote cell cycle progression by phosphorylating and inactivating the retinoblastoma protein (RB). Without wishing to be bound by theory, cyclin D1 can also function as transcriptional modulator by regulating the activity of several transcription factors and histone deacetylase (HDAC3), which can be independent of CDK4 activity.

In some embodiments, the cells are modified to overexpress the coding sequence of a cyclin D1 protein. In some embodiments, the cells are genetically modified and carry stable integrations of one or more copies of a cyclin D1 coding sequence. In some embodiments, the cells overexpress the coding sequence of a cyclin D1 protein at levels sufficient to increase proliferation and/or immortalize the cell.

In some embodiments, the cyclin D1 coding sequence is selected from any metazoan species. In some embodiments, the cyclin D1 coding sequence is from any animal, such as vertebrate and invertebrate animal species. In some embodiments, the cyclin D1 coding sequence is from any vertebrate animal species such as mammals, reptiles, birds, amphibians, and the like. In some embodiments, the cyclin D1 coding sequence is from any mammalian species such as a human, murine, bovine, porcine, poultry, and the like. In some embodiments, the coding sequence of the cyclin D1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the cyclin D1 protein is derived from Bos Taurus.

In some embodiments, increasing expression of cyclin D1 may be achieved using different approaches. In some embodiments, the expression is inducible. In some embodiments, the method comprises expressing polynucleotides comprising the coding sequence of cyclin D1. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the cyclin D1 gene involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding Cyclin D1 integrates into the cell's genome but undergoes silencing.

In the methods described herein, a polynucleotide comprising a coding sequence of cyclin D1 may encode any homolog of cyclin D1, including cyclin D1 paralogs, or a cyclin D1 protein translated from any splice variants of a cyclin D1 gene, or may comprise any mutations in the cyclin D1 gene sequence including, but not limited to nucleotide deletions, truncations, fusions, or substitutions. Mutations may be synthetic or naturally occurring.

In some embodiments, cyclin D1 refers to the cyclin D1 gene or cyclin D1 protein, or fragment or variant thereof (e.g., a cyclin D1 protein having one or more (e.g., one, two, three, four, five, six, seven, eight, nine, ten, or more amino acid substitutions, deletions or insertions as compared to a wild type cyclin D1 polypeptide)). FIG. 4B shows a plasmid map of a construct used to introduce the coding sequence of the cyclin D1 into any of the cells described herein.

In some embodiments, a cyclin D1 protein comprises an amino acid sequence having at least 80% ((e.g., at least 85%, 90, 95%, 96%, 97%, 98%, or 99%) sequence identity to the amino acid sequence of SEQ ID NO: 7. In some embodiments, the cyclin D1 protein sequence comprises an amino acid sequence of SEQ ID NO: 7.

In some embodiments, a polynucleotide encoding Cyclin D1 comprises a nucleic acid sequence having at least 80% (e.g., at least 85%, 90, 95%, 96%, 97%, 98%, or 99%) sequence identity to the nucleic acid sequence of SEQ ID NO: 8. In some embodiments, the polynucleotide encoding Cyclin D1 comprises a nucleic acid sequence of SEQ ID NO: 8.

In some embodiments, introducing the polynucleotide comprising the coding sequence of the cyclin D1 protein alone is not sufficient to induce cell proliferation and/or immortalize the cell. In such embodiments, one or more additional proteins that are capable of modulating cell proliferation can be introduced into the cell line. For example, a polynucleotide comprising the coding sequence of a TERT protein, a BMI-1 protein, a CDK4 protein, a PCG1α protein, a DKC1 protein, a YAP1 protein, or a Nanog, or a combination thereof, can be introduced into the cell line to help immortalize or expand the replicative capacity of the cell.

### 4.7.4. Peroxisome Proliferator-Activated Receptor Gamma Coactivator 1-Alpha (PGC-1α)

In some embodiments, the methods provided herein include introducing into a cell a polynucleotide encoding peroxisome proliferator-activated receptor gamma coactivator 1-alpha (PGC-1α or PGC1-alpha) or a fragment thereof. As used herein, “PGC-1α” refers to the peroxisome proliferator-activated receptor gamma coactivator 1-alpha (PGC-1α) gene or PGC-1α.

Without wishing to be bound by theory, PGC1α interacts with PPARgamma, which permits the interaction of this protein with multiple transcription factors. In some embodiments, PGC1α interacts with, and regulate the activities of, cAMP response element binding protein (CREB) and nuclear respiratory factors (NRFs). In some embodiments, PGC1α provides a direct link between external physiological stimuli and the regulation of mitochondrial biogenesis and is a major factor that regulates muscle fiber type determination.

In some embodiments, the cells are modified to overexpress the coding sequence of an PGC1α protein. In some embodiments, the cells are genetically modified and carry stable integrations of one or more copies of an PGC1α coding sequence. In some embodiments, the cells overexpress the coding sequence of PGC1α protein at levels sufficient to increase cell proliferation and/or immortalize the cell.

In some embodiments, the PGC1α coding sequence is selected from any metazoan species. In some embodiments, the PGC1α coding sequence is from any animal, such as vertebrate and invertebrate animal species. In some embodiments, the PGC1α coding sequence is from any vertebrate animal species such as mammals, reptiles, birds, amphibians, and the like. In some embodiments, the PGC1α coding sequence is from any mammalian species such as a human, murine, bovine, porcine, poultry, and the like. In some embodiments, the coding sequence of the PGC1α protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the PGC1α is derived from Bos taurus.

In some embodiments, increasing expression of PGC1α may be achieved using different approaches. In some embodiments, the expression is inducible. In some embodiments, the method comprises expressing polynucleotides comprising the coding sequence of PGC1α. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the PGC1α gene involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding PGC1α is integrated into the cell's genome but undergoes silencing.

In the methods described herein, a polynucleotide comprising a coding sequence of PGC1α may encode any homolog of PGC1α, including PGC1α paralogs, or an PGC1α protein translated from any splice variants of an PGC1α gene, or may comprise any mutations in the PGC1α gene sequence including, but not limited to nucleotide deletions, truncations, fusions, or substitutions. Mutations may be synthetic or naturally occurring.

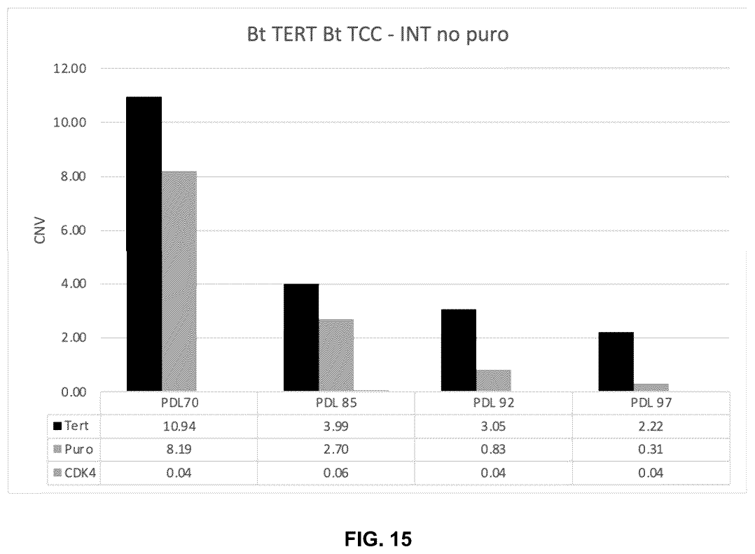

In some embodiments, PGC1α refers to the PGC1α gene or PGC1α protein, or fragment or variant thereof (e.g., a PGC1α protein having one or more (e.g., one, two, three, four, five, six, seven, eight, nine, ten, or more amino acid substitutions, deletions or insertions as compared to a wild type PGC1α polypeptide)). FIG. 15B shows a plasmid map of a construct used to introduce the coding sequence of the PGC1α protein into any of the cells described herein.

In some embodiments, a PGC1α protein comprises an amino acid sequence having at least 80% ((e.g., at least 85%, 90, 95%, 96%, 97%, 98%, or 99%) sequence identity to the amino acid sequence of SEQ ID NO: 9. In some embodiments, the PGC1α protein sequence comprises an amino acid sequence of SEQ ID NO: 9.

In some embodiments, a polynucleotide encoding PGC1α comprises a nucleic acid sequence having at least 80% (e.g., at least 85%, 90, 95%, 96%, 97%, 98%, or 99%) sequence identity to the nucleic acid sequence of SEQ ID NO: 10. In some embodiments, the polynucleotide encoding PGC1α comprises a nucleic acid sequence of SEQ ID NO: 10.

In some embodiments, introducing the polynucleotide comprising the coding sequence of the PGC1α protein alone is not sufficient to increase cell proliferation and/or immortalize the cell. In such embodiments, one or more additional proteins that are capable of modulating cell proliferation can be introduced into the cell. For example, a polynucleotide comprising the coding sequence of a TERT protein, a BMI-1 protein, a Cyclin D1 protein, a CDK4 protein, a DKC1 protein, a YAP1 protein, or a Nanog protein, or a combination thereof, can be introduced into the cell line to help immortalize or expand the replicative capacity of the cell.

### 4.7.5. Nanog

In some embodiments, the methods provided herein include introducing into a cell a polynucleotide Nanog (NANOG) or a fragment thereof. As used herein, “Nanog” refers to the Nanog gene or NANOG protein.

In some embodiments, the cells are modified to overexpress the coding sequence of an Nanog protein. In some embodiments, the cells are genetically modified and carry stable integrations of one or more copies of an Nanog coding sequence. In some embodiments, the cells overexpress the coding sequence of Nanog protein at levels sufficient to increase production and/or secretion of Nanog into the cell medium.

In some embodiments, the Nanog coding sequence is selected from any metazoan species. In some embodiments, the Nanog coding sequence is from any animal, such as vertebrate and invertebrate animal species. In some embodiments, the Nanog coding sequence is from any vertebrate animal species such as mammals, reptiles, birds, amphibians, and the like. In some embodiments, the Nanog coding sequence is from any mammalian species such as a human, murine, bovine, porcine, poultry, and the like. In some embodiments, the coding sequence of the Nanog protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of the Nanog is derived from Bos taurus.

In some embodiments, increasing expression of Nanog may be achieved using different approaches. In some embodiments, the expression is inducible. In some embodiments, the method comprises expressing polynucleotides comprising the coding sequence of Nanog. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the expression of the Nanog gene involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding NANOG integrates into the cell's genome but undergoes silencing.

In the methods described herein, a polynucleotide comprising a coding sequence of Nanog may encode any homolog of Nanog, including Nanog paralogs, or an Nanog protein translated from any splice variants of an Nanog gene, or may comprise any mutations in the Nanog gene sequence including, but not limited to nucleotide deletions, truncations, fusions, or substitutions. Mutations may be synthetic or naturally occurring.

In some embodiments, NANOG refers to the Nanog gene or NANOG protein, or fragment or variant thereof (e.g., a NANOG protein having one or more (e.g., one, two, three, four, five, six, seven, eight, nine, ten, or more amino acid substitutions, deletions or insertions as compared to a wild type NANOG polypeptide)). FIG. 15A shows a plasmid map of a construct used to introduce the coding sequence of the Nanog protein into any of the cells described herein.

In some embodiments, a NANOG protein comprises an amino acid sequence having at least 80% ((e.g., at least 85%, 90, 95%, 96%, 97%, 98%, or 99%) sequence identity to the amino acid sequence of SEQ ID NO: 11. In some embodiments, the NANOG protein sequence comprises an amino acid sequence of SEQ ID NO: 11.

In some embodiments, a polynucleotide encoding NANOG comprises a nucleic acid sequence having at least 80% (e.g., at least 85%, 90, 95%, 96%, 97%, 98%, or 99%) sequence identity to the nucleic acid sequence of SEQ ID NO: 12. In some embodiments, the polynucleotide encoding NANOG comprises a nucleic acid sequence of SEQ ID NO: 12.

In some embodiments, introducing the polynucleotide comprising the coding sequence of the NANOG protein alone is not sufficient to increase cell proliferation and/or immortalize the cell. In such embodiments, one or more additional proteins that are capable of modulating cell proliferation can be introduced into the cell line. For example, a polynucleotide comprising the coding sequence of a TERT protein, a BMI-1 protein, a CDK4, a Cyclin D1 protein, a PCG1α protein, a DKC1 protein, or a YAP protein, or a combination thereof, can be introduced into the cell line to help immortalize or expand the replicative capacity of the cell.

### 4.7.6. Dyskerin Pseudouridine Synthase 1 (DKC1)

In some embodiments, the methods provided herein include introducing into a cell a polynucleotide encoding Dyskerin Pseudouridine Synthase 1 (DKC1) or a fragment thereof. As used herein, “DKC1” refers to the Dyskerin Pseudouridine Synthase 1 (DKC1) that plays active role in telomerase stabilization and maintenance.

In some embodiments, the cells are modified to overexpress the coding sequence of an DKC1 protein. In some embodiments, the cells are genetically modified and carry stable integrations of one or more copies of an DKC1 coding sequence. In some embodiments, the cells overexpress the coding sequence of BMI-1 protein at levels sufficient to induce cell proliferation and/or immortalize the cell.

In some embodiments, the DKC1 coding sequence is selected from any metazoan species. In some embodiments, the DKC1 coding sequence is from any animal, such as vertebrate and invertebrate animal species. In some embodiments, the DKC1 coding sequence is from any vertebrate animal species such as mammals, reptiles, birds, amphibians, and the like. In some embodiments, the DKC1 coding sequence is from any mammalian species such as a human, murine, bovine, porcine, poultry, and the like. In some embodiments, the coding sequence of the DKC1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of DKC1 protein is derived from Bos taurus.

In some embodiments, increasing expression of DKC1 may be achieved using different approaches. In some embodiments, the expression is inducible. In some embodiments, the method comprises expressing polynucleotides comprising the coding sequence of DKC1. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the constructs are not integrated into the cell's genome. In some embodiments, the construct is episomal. In some embodiments, the expression of the DKC1 gene involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding DKC1 is integrated into the cell's genome but undergoes silencing.

In some embodiments, a polynucleotide comprising a coding sequence of DKC1 may encode any homolog of DKC1, including DKC1 paralogs, or any other DKC1 paralogs, or an DKC1 protein translated from any splice variants of an DKC1 gene, or may comprise any mutations in the DKC1 gene sequence including, but not limited to nucleotide deletions, truncations, fusions, or substitutions. Mutations may be synthetic or naturally occurring.

In some embodiments, DKC1 refers to the DKC1 gene or DKC1 protein, or fragment or variant thereof (e.g., a DKC1 protein having one or more (e.g., one, two, three, four, five, six, seven, eight, nine, ten, or more amino acid substitutions, deletions or insertions as compared to a wild type DKC1 protein)). FIG. 15D shows a plasmid map of a construct used to introduce the coding sequence of BMI into any of the cells described herein.

In some embodiments, an DKC1 protein comprises an amino acid sequence having at least 80% (e.g., at least 85%, 90, 95%, 96%, 97%, 98%, or 99%) sequence identity to the amino acid sequence of SEQ ID NO: 13. In some embodiments, the DKC1 protein sequence comprises an amino acid sequence of SEQ ID NO: 13.

In some embodiments, a polynucleotide encoding DKC1 comprises a nucleic acid sequence having at least 80% (e.g., at least 85%, 90, 95%, 96%, 97%, 98%, or 99%) sequence identity to the nucleotide sequence of SEQ ID NO: 14. In some embodiments, the polynucleotide encoding DKC1 comprises a nucleic acid sequence of SEQ ID NO: 14.

In some embodiments, introducing the polynucleotide comprising the coding sequence of the DKC1 protein alone is not sufficient to immortalize the cell. In such embodiments, one or more additional proteins that are capable of modulating cell proliferation can be introduced into the cell. For example, a polynucleotide comprising the coding sequence of a TERT protein, a CDK4 protein, a Cyclin D1 protein, a PCG1α protein, a YAP, or a Nanog protein, or a combination thereof, can be introduced into the cell line to help immortalize or expand the replicative capacity of the cell.

### 4.7.7. Yes Associated Protein 1 (YAP1)

In some embodiments, the methods provided herein include introducing into a cell a polynucleotide Yes Associated Protein (YAP1) or a fragment thereof. As used herein, “YAP” or “YAP1” refers to the yes associated protein that is involved in mediating growth and development. In some embodiments, YAP1 expression (RNA and/or protein) levels are modulated as described in US20200165569A1, which is herein incorporated by reference in its entirety.

In some embodiments, the cells are modified to overexpress the coding sequence of a YAP1 protein. In some embodiments, the cells are genetically modified and carry stable integrations of one or more copies of a YAP1 coding sequence. In some embodiments, the cells overexpress the coding sequence of YAP1 protein at levels sufficient to induce cell proliferation and/or immortalize the cell.

In some embodiments, the YAP1 coding sequence is selected from any metazoan species. In some embodiments, the YAP1 coding sequence is from any animal, such as vertebrate and invertebrate animal species. In some embodiments, the YAP1 coding sequence is from any vertebrate animal species such as mammals, reptiles, birds, amphibians, and the like. In some embodiments, the YAP1 coding sequence is from any mammalian species such as a human, murine, bovine, porcine, poultry, and the like. In some embodiments, the coding sequence of the YAP1 protein is derived from a species selected from any metazoan species, including without limitation, Gallus gallus, Bos taurus, Sous scrofa, Meleagris gallopavo, Anas platyrynchos, Salmo salar, Thunnus thynnus, Ovis aries, Coturnix coturnix, Copra aegagrus hircus, or Homarus americanus. In some embodiments, the coding sequence of YAP1 protein is derived from Bos taurus.

In some embodiments, increasing expression of YAP1 may be achieved using different approaches. In some embodiments, the expression is inducible. In some embodiments, the method comprises expressing polynucleotides comprising the coding sequence of YAP1. In some embodiments, the polynucleotides are ectopically expressed from constructs that are introduced into the cells, for example expressed from a plasmid, or other expression vector. In some embodiments, the constructs are integrated into the cell's genome, and the expression is driven in that manner (e.g., PhiC31 Integration Systems). In some embodiments, the constructs are not integrated into the cell's genome. In some embodiments, the construct is episomal. In some embodiments, the expression of the YAP1 gene involves electroporating a DNA, delivering a DNA complexed with a transfection vehicle, using a viral vector (e.g. retrovirus, lentivirus, adenovirus, adeno-associated virus, herpes simplex virus), and the like, or combinations thereof. In some embodiments, the expression is constitutive. In some embodiments, the expression is conditional (e.g. inducible). In some embodiments, the polynucleotide encoding YAP1 is integrated into the cell's genome but undergoes silencing.

In some embodiments, a polynucleotide comprising a coding sequence of YAP1 may encode any homolog of YAP1, including YAP1 paralogs, or any other BMI-1 paralogs, or an YAP1 protein translated from any splice variants of an YAP1 gene, or may comprise any mutations in the YAP1 gene sequence including, but not limited to nucleotide deletions, truncations, fusions, or substitutions. Mutations may be synthetic or naturally occurring.

In some embodiments, YAP1 refers to the YAP1 gene or YAP1 protein, or fragment or variant thereof (e.g., a YAP1 protein having one or more (e.g., one, two, three, four, five, six, seven, eight, nine, ten, or more amino acid substitutions, deletions or insertions as compared to a wild type YAP1 protein)). FIG. 15C shows a plasmid map of a construct used to introduce the coding sequence of BMI into any of the cells described herein.

In some embodiments, an YAP1 protein comprises an amino acid sequence having at least 80% (e.g., at least 85%, 90, 95%, 96%, 97%, 98%, or 99%) sequence identity to the amino acid sequence of SEQ ID NO: 15. In some embodiments, the YAP1 protein sequence comprises an amino acid sequence of SEQ ID NO: 15.

In some embodiments, a polynucleotide encoding YAP1 comprises a nucleic acid sequence having at least 80% (e.g., at least 85%, 90, 95%, 96%, 97%, 98%, or 99%) sequence identity to the nucleic acid sequence of SEQ ID NO: 16. In some embodiments, the polynucleotide encoding YAP1 comprises a nucleic acid sequence of SEQ ID NO: 16.

In some embodiments, introducing the polynucleotide comprising the coding sequence of the YAP1 protein alone is not sufficient to immortalize the cell. In such embodiments, one or more additional proteins that are capable of modulating cell proliferation can be introduced into the cell. For example, a polynucleotide comprising the coding sequence of a TERT protein, a CDK4 protein, a Cyclin D1 protein, a PCG1α protein, a DKC1 protein, or a Nanog protein, or a combination thereof, can be introduced into the cell line to help immortalize or expand the replicative capacity of the cell.

### 4.8. Polynucleotide Excision

In another aspect, provided herein are methods of reducing or eliminating expression of the proteins encoded by the polynucleotides introduced into the cells (e.g., bovine cells). Reducing or eliminating expression of the proteins encoded by the polynucleotides introduced into the cells (e.g., bovine cells) provides temporal control over the impact of over exogenous protein levels. In one embodiments, eliminating expression of the protein (e.g., TERT or a protein that is capable of modulating cell proliferation) is to eliminate the polynucleotides encoding the protein, thereby ensuring temporal control over the proteins impact on the cell. In some embodiments, eliminating the polynucleotides encoding the proteins enables the cell (or a population derived therefrom) to meet certain safety criteria. In some embodiments, meeting safety criteria includes removing the polynucleotide backbone (e.g., antibiotic resistance gene). In some embodiments, meeting safety criteria includes the coding sequences from the polynucleotide backbone (e.g., antibiotic resistance gene, polynucleotide encoding TERT, polynucleotide encoding a gene of interest, or a combination thereof).

In some embodiments, the method includes reducing or eliminating expression of TERT. In some embodiments, the method includes reducing or eliminating expression of the at least first protein that is capable of modulating cell proliferation. In some embodiments, the method includes reducing or eliminating expression of TERT and expression of the at least a first protein that is capable of modulating cell proliferation.

In some embodiments, reducing or eliminating expression is effected by introducing into the cell: an effector protein. In some embodiments, the effector protein is selected from a nuclease, a recombinase, and an integrase.

In some embodiments, the effector protein is a nuclease. In some embodiments, the nuclease induces a genetic perturbation in the integrated polynucleotide (e.g., the polynucleotide encoding TERT), whereby the genetic perturbation disrupts expression of the protein encoded by the polynucleotide (e.g., TERT). In some embodiments, the nuclease is a Cas9 endonuclease, a Zinc-finger nuclease, a TALE-nuclease, or a mega-nuclease. In some embodiments, the nuclease is a Cas9 endonuclease.

In some embodiments, the effector protein is a recombinase. In some embodiments, the recombinase excises, inverts, or translocates, or a combination thereof, the integrated polynucleotide, whereby excising, inverting, or translocating disrupts expression of the protein encoded by the polynucleotide. In some embodiments, the recombinase is a CRE protein or a Flippase protein. In some embodiments, the recombinase is a CRE protein.

In some embodiments, the effector protein is an integrase. In some embodiments, the integrase excises, inverts, or translocates, or a combination thereof, the integrated polynucleotide, whereby excising, inverting, or translocating disrupts expression of the protein encoded by the polynucleotide. In some embodiments, the integrase is a serine integrase. In some embodiments, the integrase is PhiC31.

In some embodiments, the effector protein disrupts expression of the polynucleotide encoding TERT. In some embodiments, the effector protein disrupts expression, of the polynucleotide encoding TERT at the genomic location at which the polynucleotide encoding TERT is incorporated.

In some embodiments, the effector protein disrupts expression of the at least a first polynucleotide encoding a protein that is capable of modulating cell proliferation. In some embodiments, the effector protein disrupts expression of the at least a first polynucleotide encoding a protein that is capable of modulating cell proliferation at the genomic location at which the polynucleotide encoding a protein that is capable of modulating cell proliferation is incorporated.

In some embodiments, the polynucleotide encoding TERT comprises recognition sites flanking the polynucleotide sequence encoding TERT.

In some embodiments, the at least a first polynucleotide encoding a protein that is capable of modulating cell proliferation comprises recognition sites flanking the polynucleotide sequenced encoding the protein that is capable of modulating cell proliferation.

In some embodiments, the recognition sites are selected from lox66, lox71, attB, attP, attL, attR, and FRT. In such cases, the method includes introducing the effector protein that recognizes the recognition site (e.g., CRE-lox; PhiC31-attB, and Flippase-FRT).

In some embodiments, the effector protein is CRE and CRE (or a polynucleotide expressing CRE) is introduced into a cell where one or more of the exogenous polynucleotides includes recognition sites (e.g., flanking a polynucleotide to be excised, inverted, or translocated), wherein disrupting expression occurs by excising, inverting, or translocating the polynucleotide sequence (e.g., the polynucleotide sequence flanked by the loxP site). In some embodiments, the recognition site is a lox site. In one embodiment, the lox site is selected from lox66 and lox71. In one embodiment, the lox site is a lox66. In one embodiment, the lox site is a lox71. In one embodiment, a polynucleotide includes two lox sites. For example, a polynucleotide encodes a first lox site (e.g., lox66) and a second lox site (e.g., lox71), where the directionality of the lox sites determines the polynucleotide sequence flanked by the lox sites is inverted or deleted.

### 4.9. Bovine Cells

In some embodiments, the bovine cells are non-myogenic cells. In some embodiments, the bovine cells are non-myogenic cells but harbor myogenic capacity. In some embodiments, the non-myogenic cells are fibroblasts, mesenchymal stem cells, and chondrocytes.

In some embodiments, the bovine cells are myogenic cells. In some embodiments, the myogenic cells are myoblasts, myocytes, satellite cells, side population cells, muscle derived stem cells, mesenchymal stem cells, myogenic pericytes, or mesoangioblasts. In some embodiments, the cells are non-myogenic cells.

In embodiments, the bovine cells are taken from a primary bovine source, such as bovine muscle, skin or other bovine tissue. In other embodiments, the bovine cells are taken from a master cell bank or have otherwise been previously cultured and stored. The bovine cells which are introduced into the culture are not immortalized but may be immortalized by the method described herein. In embodiments, the bovine cells that are transfected with the polynucleotides encoding or otherwise caused to express TERT and the protein capable of modulating cell proliferation have undergone 5, 10, 15, 20, 25, 30, 35, or 40 doublings since isolation from primary tissue or passaging or sampling from a source of bovine cells.

### 4.10. Cultivation Infrastructure

In some embodiments, a cultivation infrastructure may be a tube, a cylinder, a flask, a petri-dish, a multi-well plate, a dish, a vat, a roller bottle, an incubator, a bioreactor, an industrial fermenter and the like.

In some embodiments, a cultivation infrastructure can be of any scale, and support any volume of cellular biomass and culturing reagents. In some embodiments, the cultivation infrastructure ranges from about 10 μL to about 100,000 L. In some embodiments, the cultivation infrastructure is about 10 μL, about 100 μL, about 1 mL, about 10 mL, about 100 mL, about 1 L, about 10 L, about 100 L, about 1000 L, about 10,000 L, or even about 100,000 L or even about 150,000 L, or greater.

In some embodiments, the cultivation infrastructure comprises a substrate. In some embodiments, a cultivation infrastructure may comprise a permeable substrate (e.g. permeable to physiological solutions) or an impermeable substrate (e.g. impermeable to physiological solutions).

In some embodiments, the cultivation infrastructure comprises a primary substrate, which can be a flat, concave, or convex substrate. In some embodiments, the cultivation infrastructure further comprises a secondary substrate, either introduced, or autologous, to direct cellular growth between the substrates, e.g. to direct attachment, proliferation and hypertrophy of cells on a plane perpendicular to the primary substrate.

In some embodiments, the cultivation infrastructure comprises a hydrogel, a liquid cell culture media, or soft agar.

In some embodiments, the cultivation infrastructure does not comprise a substrate to which cells can adhere. In some embodiments, the cultivation infrastructure comprises a suspension culture, e.g. supporting the growth of a self-adhering biomass, or single-cell suspension in a liquid medium.

In some embodiments, the cultivation infrastructure comprises adherent cells (i.e. those cells that adhere to a substrate). In some embodiments, the cultivation infrastructure comprises non-adherent cells (i.e. those cells that do not adhere to a substrate). In some embodiments, the cultivation infrastructure comprises both adherent and non-adherent cells.

Non-limiting examples of cultivation infrastructure include those described in U.S. Patent Publication Nos. 2020/0110347, 2022/0056394, and 2021/014031, which are herein incorporated by reference in their entireties.

### 4.11. Nucleic Acids/Vectors

In another aspect, provided herein are polynucleotides comprising coding sequences of TERT, any of the proteins capable of modulating cell proliferation, or a combination thereof. In some embodiments, a construct (i.e., a vector) includes any of the polynucleotides described herein. In some embodiments, any of the vectors described herein can be an expression vector. In some embodiments, an expression vector can include one or more promoter sequences (e.g., any of the promoter sequences described herein) operably linked to a coding sequence of any of the proteins capable of modulating cell proliferation described herein, TERT, or a combination thereof. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some embodiments, a vector includes sufficient cis-acting elements that supplement expression where the remaining elements needed for expression can be supplied by the host cell (e.g., the cell line).

In some embodiments, a vector includes a polynucleotide comprising a coding sequence of a protein (e.g. TERT). In some embodiments, a vector includes a polynucleotide comprising a first coding sequence of TERT and a second coding sequence of a protein capable of modulating cell proliferation. In some embodiments, a vector (e.g., a construct) includes a polynucleotide comprising a first coding sequence of TERT, a second coding sequence of a first protein capable of modulating cell proliferation, and a third coding sequence of a second protein capable of modulating cell proliferation. In such embodiments where the construct includes two or more coding sequences, each of the two or more coding sequences are operably linked to a promoter sequence or to another coding sequence via a self-cleaving polypeptide or IRES. As used herein, the term “operably linked” is well known in the art and refers to genetic components that are combined such that they carry out their normal functions. For example, a coding sequence is operably linked to a promoter when its transcription is under the control of the promoter. In another example, a coding sequence can be operably linked to other coding sequences by a self-cleaving 2A polypeptide or an internal ribosome entry site (IRES). In such cases, the self-cleaving 2A polypeptide allows the second coding sequence to be under the control of the promoter operably linked to the first coding sequence. In some cases, the coding sequences described herein can be operably linked to any other coding sequence described herein using a self-cleaving 2A polypeptide or IRES.

In some embodiments, a vector includes a polynucleotide comprising a coding sequence of a single protein capable of modulating cell proliferation or fragment thereof. In some embodiments, a vector includes a polynucleotide comprising a first coding sequence of a first protein capable of modulating cell proliferation and a second coding sequence of a second protein capable of modulating cell proliferation. In some embodiments, a vector (e.g., a construct) includes a polynucleotide comprising a first coding sequence of a first protein capable of modulating cell proliferation, a second coding sequence of a second protein capable of modulating cell proliferation, and a third coding sequence of a third protein capable of modulating cell proliferation. In such embodiments where the construct includes coding sequences for two or more protein capable of modulating cell proliferation, each of the two or more coding sequences are operably linked to a promoter sequence or to another coding sequence via a self-cleaving polypeptide or IRES. In another example, a coding sequence can be operably linked to other coding sequences by a self-cleaving 2A polypeptide or an internal ribosome entry site (IRES). In such cases, the self-cleaving 2A polypeptide allows the second coding sequence to be under the control of the promoter operably linked to the first coding sequence. In some cases, the coding sequences described herein can be operably linked to any other coding sequence described herein using a self-cleaving 2A polypeptide or IRES.

Non-limiting examples of vectors that can be used in the methods described herein are as shown in FIG. 3, FIGS. 4A-4B, and FIG. 15A-15D.

Also provided herein are a set of vectors that include two or more vectors

In some embodiments, a coding sequence of any polynucleotides described herein is operably linked to a promoter. In some embodiments, the promoter is a tissue-specific promoter. In some embodiments, the tissue-specific promoter is a muscle-specific promoter. In some embodiments, the muscle-specific promoter is selected from the group consisting of: skeletal β-action, myosin light chain 2a, dystrophin, SPc-512, muscle creatine kinase, and synthetic muscle promoters. In some embodiments, the promoter is a constitutively active promoter. In some embodiments, the promoter is selected from the group consisting of: EF1 (e.g., EF1alpha), PGK, CMV, RSV, and β-actin. In some embodiments, the promoter is a EF1 (e.g., EF1alpha) promoter. In some embodiments, the promoter is a PGK promoter.

In some embodiments, the vector comprises a selectable marker. In some embodiments, the selectable marker is an antibiotic resistance protein that confers antibiotic resistance when expressed. In such embodiments, the methods provided herein include a selecting step that comprises contacting the cells with an antibiotic under conditions sufficient to enable selection of the transduced cells.

In some embodiments, the selectable marker is a fluorophore. In such embodiments, the methods provided herein include a selecting step that comprises performing fluorescence activated cell sorting (FACS).

In some embodiments, a vector system is used to integrate a polynucleotide comprising a coding sequence of TERT, any one or more of the proteins capable of modulating cell proliferation, or a combination thereof, into the genome of a cell line (e.g., any of the cell lines described herein). In some embodiments, the vector system used for integration is a vector phiC31 Integrase Vector System. Additional non-limiting examples of vectors systems that can be used to integrate a polynucleotide (e.g., any of the polynucleotides described herein) into the genome of a bovine cell (e.g., any of the bovine cell described herein) include: a sleeping beauty transposon system (as described in U.S. Pat. No. 7,985,739), a piggyBac transposition system (as described in US20090042297), CRISPR/Cas-mediated knockin, TALEN-mediated knockin, and viral vector-mediated integration. In such embodiments where integration is mediated via a viral vector, non-limiting examples of viral vectors include adenovirus, adeno-associated virus, lentivirus, and a retrovirus (e.g., a gamma-retrovirus).

In some embodiments, the polynucleotide encoding TERT and the at least a first polynucleotide encoding a first protein that is capable of modulating cell proliferation are on the same contiguous polynucleotide (e.g., the same vector). In some embodiments, the polynucleotide encoding TERT and the at least a first polynucleotide encoding a first protein that is capable of modulating cell proliferation are on separate polynucleotides (e.g., separate vectors).

In some embodiments, the polynucleotide encoding TERT include integrase recognition sites. In some embodiments, the integrase recognition sites are used to mediate incorporation (integration) of the polynucleotide (e.g., polynucleotide encoding TERT) into the genome of the cell. In some embodiments, the polynucleotide encoding TERT include recognition sites. In some embodiments, the recognition sites are used to reduce or eliminate expression of the protein encoded by the polynucleotide (e.g., TERT). In such cases, the recognition site can be recognized by an effector protein that mediates reduce or eliminate expression of the protein encoded by the polynucleotide (e.g. TERT).

In some embodiments, the polynucleotide encoding the at least a first protein capable of modulating the cell proliferation include integrase recognition sites. In some embodiments, the integrase recognition sites are used to mediate incorporation (integration) of the polynucleotide (e.g., polynucleotide encoding a protein capable of modulating cell proliferation) into the genome of the cell. In some embodiments, the polynucleotides encoding the at least a first protein capable of modulating the cell proliferation include recognition sites. In some embodiments, the recognition sites are used to reduce or eliminate expression of the protein encoded by the polynucleotide (e.g., a protein capable of modulating cell proliferation). In such cases, the recognition site can be recognized by an effector protein that mediates reduce or eliminate expression of the protein encoded by the polynucleotide (e.g. a protein capable of modulating cell proliferation).

### 4.12. Methods of Transducing Cells

Methods of introducing nucleic acids and expression vectors into a cell (e.g., an immortalized cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral, retroviral, and lentiviral transduction), lipid nanoparticle (LNP) transfection, and nanoparticle transfection.

### 4.13. Kits

Also provided herein are kits comprising any of the cell lines, any of the cells derived from the cell lines, any of the polynucleotides described herein (e.g., any of the coding sequence of any one or more of the growth factor ligands described herein, any one or more of the growth factor ligands described herein, any of the accessory protein described herein, or a combination thereof). In some embodiments, the kit includes instructions for performing any of the methods described herein.

### 4.14. Cells/Cell Lines

Also provided herein are cell line(s) for cultured food production In some embodiments, the cell line(s) are capable of self-renewal. In some embodiments, the cell line(s) are immortalized cell line(s). In some embodiments, the cell lines are then differentiated to cell types of interest (e.g., myogenic cells).

Also provided herein are immortalized cells (e.g., any of the immortalized cells described herein). In some embodiments, the immortalized cells are non-myogenic cells. In some embodiments, the immortalized cells are fibroblasts. In some embodiments, the immortalized cells comprise any of the nucleic acids described herein that encode any of the myogenic regulatory factors described herein.

Also provided herein are cells comprising any of the polynucleotides described herein that include TERT, any one or more of the proteins capable of modulating cell proliferation described herein, or a combination thereof.

Also provided herein are cells that include at least one integrase recognition site integrated into the genome. In some embodiments, the method includes integrating at least a first integrase recognition site prior to integrating the polynucleotide encoding TERT and/or prior to integrating the polynucleotide encoding a protein capable of modulating cell proliferation. Non-limiting examples of methods that can be used to insert the integrase recognition site into the genome include CRISPR/Cas9-mediated knockin, viral-mediated integration (e.g., lentivirus or retrovirus), non-viral mediated integration, Zinc-finger-mediated knockin, and TALE-mediated knockin.

Also provided herein are cells derived from the cell line(s). Non-limiting examples of cells derived from the immortalized cells (e.g., using the methods described herein) include myoblasts, myotubes, multinucleated myotubes, satellite cells, skeletal muscle fibers, or any combination thereof.

In some embodiments, the cell or cell line is from bovine.

In some embodiments, the cell line is derived from Bos taurus.

In some embodiments, an immortalized cell is not a stem cell (e.g., a muscle stem cell or a muscle satellite cell). In some embodiments, an immortalized cell is not a pluripotent stem cell (e.g., an embryonic stem cell or an induced pluripotent stem cell).

Also provided herein are cell banks comprising immortalized cell lines (e.g., immortalized fibroblast cells lines) generated according to the methods described herein.

In some embodiments, the population of cells are selected from fibroblasts, myofibroblasts, and myogenic cells, or a combination thereof.

In some embodiments, the cells are not natively myogenic (e.g., are non-myogenic cells such as fibroblasts or non-myogenic stem cells that are cultured to become myogenic cells (e.g., in suspension culture or in the cultivation infrastructure)).

In some embodiments, the cells are non-myogenic, and such non-myogenic cells can be programmed to be myogenic, for example the cells may comprise fibroblasts modified to express one or more myogenic transcription factors. In some embodiments, the myogenic transcription factors include MYOD1, MYOG, MYF5, MYF6, PAX3, PAX7, paralogs, orthologs, and genetic variants thereof. In some embodiments, the cells are modified to express one or more myogenic transcription factors as described in a PCT publication, WO/2015/066377, which is herein incorporated by reference in its entirety.

In some embodiments, the cells are genetically modified to inhibit a pathway, e.g. the HIPPO signaling pathway. Exemplary methods to inhibit the HIPPO signaling pathway as described in a PCT Application No. PCT/US2018/031276, which is herein incorporated by reference in its entirety.

In some embodiments, the cells are modified to express telomerase reverse transcriptase (TERT) and/or inhibit cyclin-dependent kinase inhibitors (CKI). In some embodiments, the cells are modified to express TERT and/or inhibit cyclin-dependent kinase inhibitors as described in a PCT publication, WO 2017/124100, which is herein incorporated by reference in its entirety.

In some embodiments, the cells are modified to express glutamine synthetase (GS), insulin-like growth factor (IGF), and/or albumin. Exemplary methods of modifying cells to express GS, IGF, and/or albumin are described in a PCT Application No. PCT/US2018/042187 which is herein incorporated by reference in its entirety.

In some embodiments, the cell-based meat product has various characteristics. Exemplary characteristics of the cell-based meat are described in U.S. application Ser. No. 17/033,635 and PCT Application No. PCT/US2021/016681, which are herein incorporated by reference in their entireties.

In some embodiments the cells are genetically edited, modified, or adapted to grow without the need of specific ingredients including specific amino acids, carbohydrates, vitamins, inorganic salts, trace metals, TCA cycle intermediates, lipids, fatty acids, supplementary compounds, growth factors, adhesion proteins and recombinant proteins.

In some embodiments, the cells may comprise any combinations of the modifications described herein.

### 4.15. Summary of Experimental Observations

Applicant evaluated methods for immortalizing bovine cells, which are known to senesce despite efforts in the field to immortalize. FIG. 2 shows a non-limiting workflow for assessing immortalization in bovine cells. In particular, Applicant evaluated cell proliferation (i.e., replicative capacity) in bovine cells engineered to express TERT and at least a first protein that is capable of modulating cell proliferation. Applicant demonstrated that introducing a polynucleotide encoding telomere reverse transcriptase (TERT); and at least a first polynucleotide encoding a protein that is capable of modulating cell proliferation into a bovine cell enables population doubling level (PDL) greater than 100 whereas wild type bovine cells have a PDL of about 54. This data demonstrated a method by which the replicative capacity of bovine cells could be increased (i.e., extended) such that a culture of bovine cells could produce enough cells for a cell-based meat product suitable for consumption.

Applicant also assessed the bovine cell lines for integration (using copy number) and mRNA expression of one or more of the genes of interest (e.g., one or more proteins that are capable of modulating cell proliferation). Surprisingly, Applicant found that the bovine cells containing the polynucleotides (e.g., polynucleotides encoding TERT and polynucleotides encoding CDK4 and cyclin D1) exhibit an increase in PDL compared to controls (i.e., bovine cells that do not include a polynucleotide encoding TERT and a polynucleotide encoding a protein that is capable of modulating cell proliferation) despite integration of only the polynucleotide encoding TERT. Moreover, Applicant found that integration one or both of the polynucleotide encoding telomere reverse transcriptase (TERT) and at least a first polynucleotide encoding a first protein that is capable of modulating cell proliferation was not necessary in order to increase (i.e., extend) replicative capacity of the bovine cells.

Overall, this work demonstrated the ability to increase (i.e., extend) the replicative capacity of bovine cells such that the cells reach population doubling levels (PLDs) greater than 100. Moreover, this data showed that integration of polynucleotides encoding the genes of interest into the genome of the bovine cell is not required to induce the immortalization (i.e., reach PDL greater than 100). These findings are important because manufacturing cells suitable for consumption requires cell line adaption into particular culture formats (i.e., suspension) and cell culture media that require vast amounts of bovine cells, which is not feasible given the methods available in the art. The engineered cells provided enable PDL greater than 100 thereby increasing the supply of bovine cells that can be used to produce the cell based meat products described herein.

### 5. EXAMPLES

### 5.1. Experimental Procedures/Methods

### 5.1.1. Integration of Gene of Interest

In order to generate cells lines with integrated polynucleotides, a PhiC31 Integrase Expression Plasmid system was used (System Biosciences, Cat No. FC200PA-1). Briefly, genes of interest (e.g., coding sequence of TERT, BMI-1, CDK4, cyclin D1, a PCG1α, Nanog, DKC1, and/or YAP (see FIG. 3, FIGS. 4A-4B, and FIGS. 15A-15D) were cloned into a PhiC31 integrase expression plasmid. Cell lines were transfected with the plasmid containing the coding sequences of the gene(s) of interest (and a plasmid containing the coding sequence of an integrase (PhiC31) to integrate the coding sequences into the genome. Cell lines with integrated plasmids were selected using puromycin (and/or FACS) and assessed for transgene expression and/or fluorescent marker expression. Cell lines exhibiting stable expression were selected for further analysis.

### 5.1.2. Excision of Integrated Plasmid Backbone

In order to remove the plasmid backbone from the cell line, a CRE recombinase was introduced into the cells. In particular, mRNA encoding CRE was added at various concentrations to the bovine 4C cells depending on cell density and/or total cell numbers. Bovine 4C cells were cultured for two passages before gDNA was harvested and backbone removal was assessed using digital droplet PCR (ddPCR).

### 5.1.3. Transfection of Genes of Interest

In order to transiently transfect cell lines with polynucleotides encoding genes of interest, genes of interest (e.g., sequence of TERT, BMI-1, CDK4, cyclin D1, a PCG1α, and Nanog) were cloned into an expression plasmid and transfected into cells using lipofectamine. In particular, bovine 4C cells seeded in a 6-well plate at 300,000 cells/well 24 hours prior to transfection. Cells were transfected using the Lipofectamine LTX protocol with 1.5 ug total of plasmid DNA encoding genes of interest and 3.5 ug of plasmid DNA encoding Integrase (in cases where integrase was included in the transfection). The full transfection protocol was performed as follows:
Day 0: Seed cells

Seed cells at 300,000 cells/well in a 6 well plate 24 h before transfection.
Day 1: Transfect cells
1. Dilute 5 ug DNA in Optimem, then add Plus Reagent. Mix well
2. Add Lipofectamine LTX to diluted DNA mix at a 1:1 ratio.
3. Incubate at RT for 10-15 min.
4. Add DNA-lipid complex to cells dropwise.
5. Incubate for 24 h
Day 2
1. Aspirate media. Rinse 1-2× with PBS.
2. Change media or passage cells
Day 3: Visualization

Analyze cells for transfection efficiency via flow cytometry to detect fluorescent tags.

### 5.1.4. Assessment of Cell Proliferation

Cells (i.e., cell lines (i.e., cell with integrated polynucleotides encoding one or more genes of interest) and/or cells transiently transfected with one or more genes of interest) were cultured until population doubling level (PDL) 100 before being transitioned to suspension culture. Cells were cultured at 5% O₂, 5% CO₂, and 39° C. degrees in DMEM/F12 containing, 20% FBS (v/v), 2% CS (v/v), and 10 ng/ml FGF2 (ME58) as adherent cultures on non-coated flasks. PDL was calculated by PDLf=PDLi+PD, where PD=(LN(Yf/Yi)/LN(2)); PDLi=previously calculated PDL; PDLf=final calculated PDL; Yi=initial population of cells; and Yf=final population of cells.

### 5.1.5. Assessment of Myogenicity

Myogenicity can be assessed using qRT-PCR (real-time quantitative reverse transcription). Messenger RNA (mRNA) is isolated from cells to examine gene expression with probes specifically designed to amplify select target genes to characterize cell lines. Identical quantity of mRNA is reverse transcribed to generate cDNA. Each cDNA is submitted to quantitative PCR (qPCR) to assess the expression of myogenic factors relative to a housekeeping gene. Expression of MyoD, MyoG, and/or MyHC1e indicate myogenic cells. Additionally, high levels of MyHC1e are indicative of cells that can mature to form myotubes.

Myogenicity can be assessed using immunohistochemistry. Cells are seeded in a 96-well plate at a low density (5000-10,000 cells/cm2) to allow cells to grow in the presence or absence of different small molecule combinations. After 2 days of media exposure, cells are fixed with 4% paraformaldehyde (PFA) and washed. Cells are permeabilized with 0.05% PBS-T (triton-x), blocked with normal goat serum (source) and are incubated with antibodies, and subsequently with secondary antibodies.

### 5.2. Example 1: Assessment of Population Doubling Level of Bovine 4C Cell Lines Compared to Controls

This experiment was designed to evaluate population doubling levels (PDLs) of bovine cell lines comprising a polynucleotide encoding telomere reverse transcriptase (TERT) and either a polynucleotide encoding BMI-1 or a polynucleotide encoding CDK4 and Cyclin D1 (referred to herein as “CC”).

For these experiments, polynucleotides encoding TERT and polynucleotides encoding BMI-1 (FIG. 3) or CDK4 and Cyclin D1 (FIGS. 4A and 4B) were integrated into bovine 4C cells according to the methods described herein and subjected to two rounds of puromycin selection. In particular, the constructs were transfected into the 4C bovine cells at PDL 23. Population doubling levels (PDLs) for each cell line was assessed and compared to control 4C bovine cells.

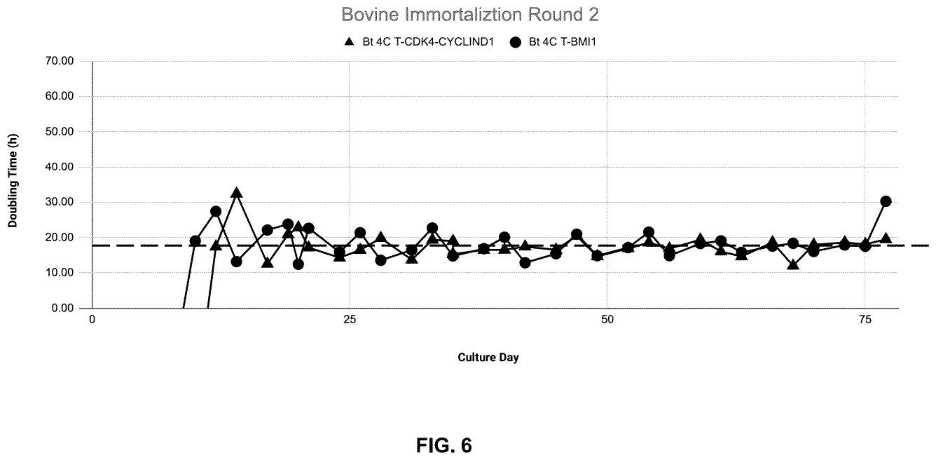

Surprisingly, as shown in FIG. 5, wild type 4C bovine cells (i.e., cells that do not include a polynucleotide encoding TERT or a polynucleotide encoding BMI-1/a polynucleotide encoding CDK4-cyclin D1) had a PDL of 54 whereas the 4C bovine cells containing either a polynucleotide encoding TERT and a polynucleotide encoding BMI-1 (Bt 4C btTERT_Bt BMI-1) or a polynucleotide encoding TERT and a polynucleotide encoding CDK4-cyclin D1 (Bt 4C btTERT_Bt CC (CDK4-cyclin D1) each had a PDL of greater than 110. This data shows that btTERT_Bt BMI-1 and btTERT_Bt CC extends proliferation capacity of 4C bovine cells.

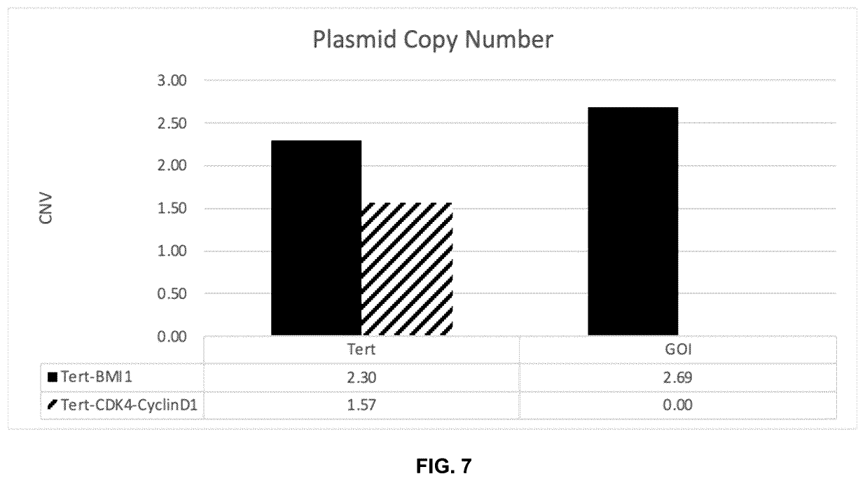

As shown in FIG. 6, population doubling time (hours) was about 18 hours for the bovine 4C cell line containing the btTERT_Bt BMI-1 polynucleotides and was about 18 hours for the bovine 4C cell line containing the btTERT_Bt CC (CDK4-cyclin D1) polynucleotides. However, primary cells generally exhibit a doubling time of 22 hours. The dual transfection of btTERT_Bt BMI-1 or btTERT_Bt CC decreased doubling time of 4C bovine cells by 4 hours.

Taken together, this data showed that the dual transfection of TERT and BMI-1 or TERT and CC (CDK4 and cyclin D1) induces cell proliferation and decreases doubling time in a bovine 4C cell line, with PDLs exceeding 100. The dual expression also causes cells to overcome senescence.

### 5.3. Example 2: Characterization of Bovine 4C Cell Lines

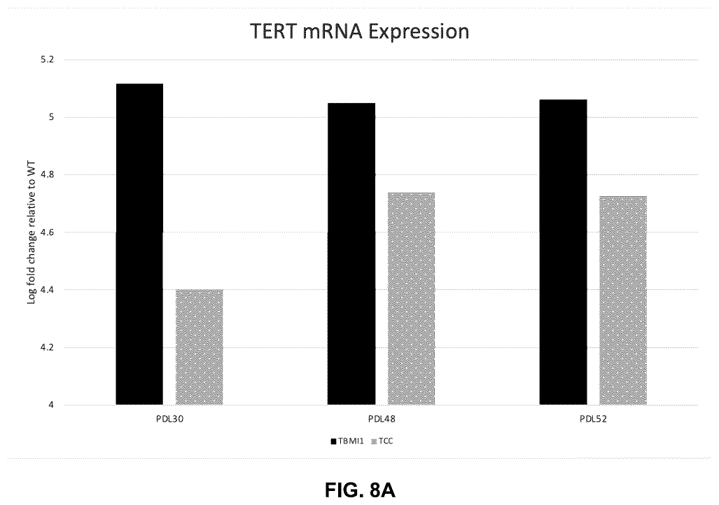

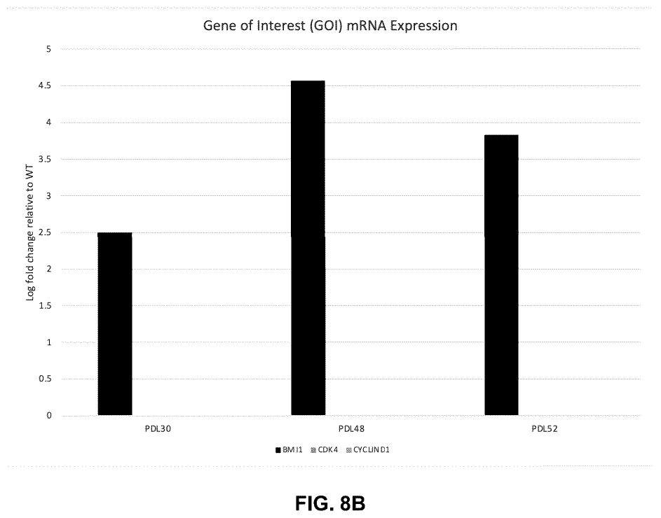

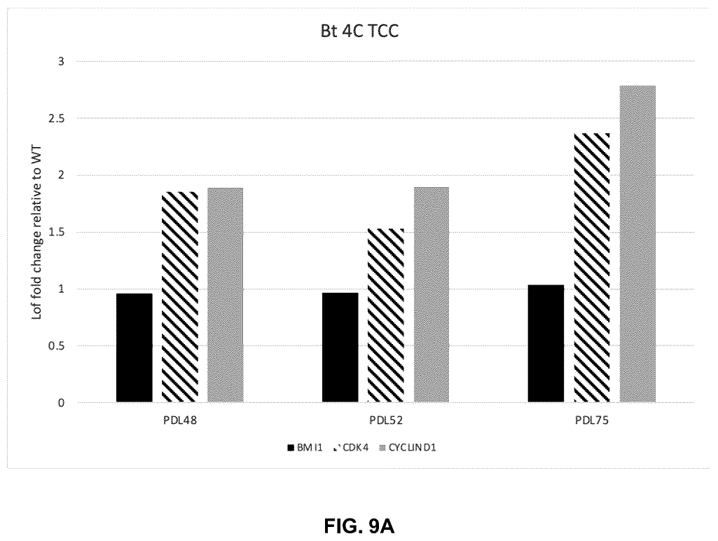

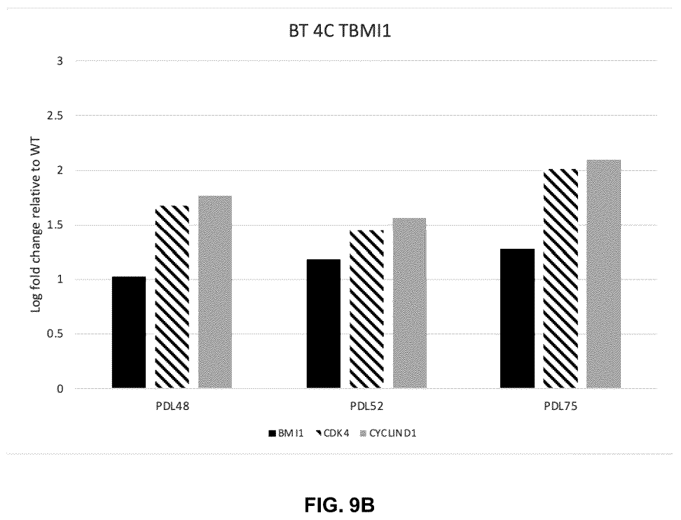

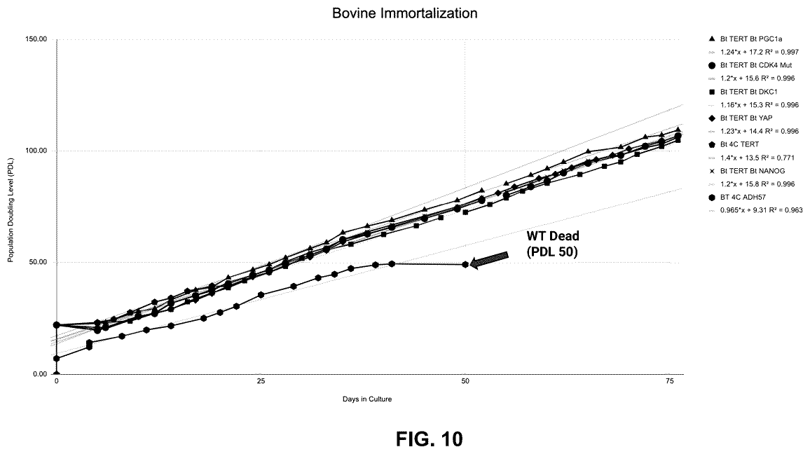

Using the cell lines from example 1, this experiment was designed to assess mRNA integration (copy number) (FIG. 7), mRNA expression levels of genes of interest encoded in exogenous polynucleotides encoded in the cell (FIGS. 8A-8B), and endogenous gene expression (FIGS. 9A-9B). These experiments were completed as described herein.

To determine plasmid copy number, 4C cells were cultured in ME58 and cell pellets were immediately frozen upon collection after 2 puromycin selections steps. For selection steps, cells were grown in culture media supplemented with 0.75 μg/mL puromycin for 5 days. gDNA was extracted and copy number was quantified via ddPCR. Data shown in FIG. 7 is presented as copy number relative to a reference gene (PGK1). 4C bovine cells transfected with btTERT_Bt BMI-1.

Interestingly, TERT plasmid copy number in btTERT_Bt BMI-1 transfected cells was higher compared to btTERT_Bt CC transfected 4C bovine cells (FIG. 7). When BMI-1 and CC integration was examined (GOI), BMI-1 integrated into the genome of the 4C bovine cells. However, CC (CDK4 and Cyclin D1) mRNA integration was not detected in btTERT_Bt CC transfected 4C bovine cells, despite the expectation that both TERT and CC would integrate into the genome.

FIGS. 8A-8B shows bovine 4C cell lines transfected with codon optimized Bt-CDK4-CyclinD1 or Bt-BMI1 and GOI+ populations and enriched via 2 puromycin selection steps. Two passages after selection (PDL 38) cell pellets were frozen, RNA was extracted, reverse transcribed, and relative mRNA transcript expression was quantified using qPCR. ΔCt was calculated using the following formula: ΔCt=Ct,GOI−Ct,GAPDH. Relative mRNA transcript expression is shown as ΔΔCT means (n=1) where ΔΔCt=ΔCt, GOI−ΔCt,4C (wildtype, WT).

QPCR analysis showed that both cell lines expressed TERT after transfection, 2 rounds of puromycin selection, and continued cultivation (PD30, 48, and 52) Interestingly, btTERT_Bt BMI-1 transfected cells consistently expressed higher levels of TERT compared to btTERT_Bt CC transfected cells across PDLs. (FIG. 8A). Transcript expression analysis confirmed that btTERT_Bt BMI-1 bovine 4C cell line expressed the exogenous BMI-1 transcript (FIG. 8B). Surprisingly, the bovine 4C cell line transfected with CC (CDK4-Cyclin D1) did not express the exogenous CDK4 or cyclin D1, thereby providing support that the polynucleotide encoding CC (CDK4-cyclin D1) did not integrate. This data also suggests that sustained expression of CC is not needed to induce immortalization in the cells.

FIGS. 9A-9B shows endogenous gene expression for bovine 4C cells stably transfected with codon optimized Bt-CDK4-CyclinD1 or Bt-BMI1 and GOI+populations and enriched via 2 puromycin selection steps. Two passages after selection (PDL 38) cell pellets were frozen, RNA was extracted, reverse transcribed, and mRNA expression quantified via qPCR. Data are ΔΔCT means (n=1) where ΔΔCt=ΔCt,GOI−ΔCt,4C (wildtype, WT). ΔCt was calculated using the following formula: ΔCt GOI−ΔCt,GAPDH. Endogenous gene expression was measured at population doubling levels (PDLs) 48, PDL 52, and PDL 75.

As shown in FIG. 9A, bovine 4C cells stably transfected with polynucleotides encoding TERT and CC (CDK4-cyclin D1) exhibited endogenous gene expression for BMI-1, CDK4 and Cyclin D1. As shown in FIG. 9B, bovine 4C cells stably transfected with polynucleotide encoding TERT and CC (CDK4 and Cyclin D1) exhibited endogenous gene expression for BMI-1, CDK4, and Cyclin D1. Both FIG. 9A and FIG. 9B suggest that the stable transduction was able to induce endogenous gene expression. In the case of FIG. 9A showing the data for cells stably transfected with TERT and CC (CDK4-cycylin D1), this is a surprising result because despite no detectable integration endogenous gene expression was similar to the controls. This suggests that integration of one or both of TERT and CC (or BMI-1) is not required to immortalize the bovine 4C cells. This is important because it eliminates having to remove an integration event for regulatory purposes, for example.

Overall, this data shows that when introducing TERT and CC (CDK4 and Cyclin D1) into bovine 4C cells integration of CC is not required to expand the replicative capacity of the bovine 4C cells. In addition, the data shows that when introducing TERT and BMI into bovine 4C cells integration of both polynucleotides provide robust expansion of the replicative capacity of the bovine 4C cells.

### 5.4. Example 3: Characterization of Bovine 4C Cell Lines Following Transduction with TERT and One or Genes of Interest (GOI)

In this example, bovine 4C cell lines were assessed following transduction with TERT and one or more genes of interest (e.g., CDK4, NANOG, PGC1a, YAP, DKC1, or CDK4-CyclinD1). In particular, population doubling levels (PDLs) measurements and gene expression analysis were assessed at various times following the transduction.

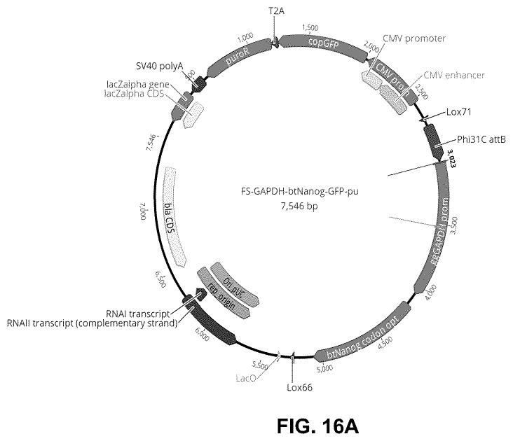

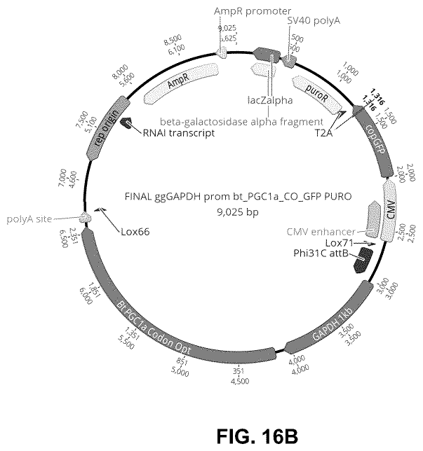

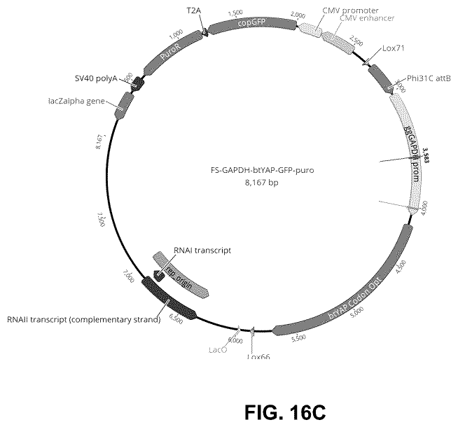

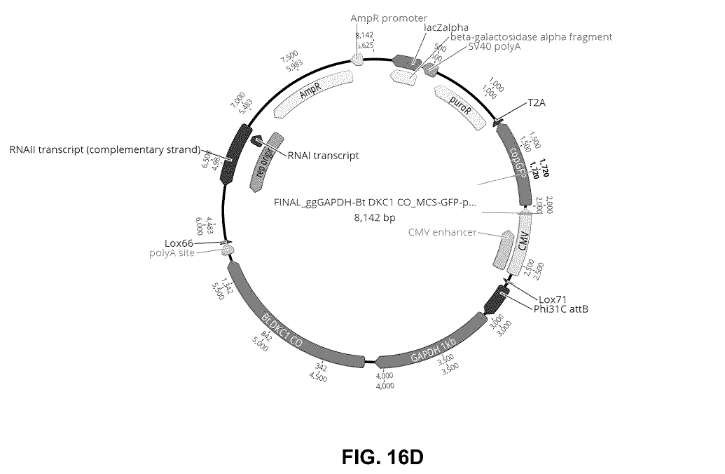

For these experiments, polynucleotides encoding TERT and polynucleotides encoding CDK4 (FIG. 4A), Nanog (FIG. 16A), PGC1a (FIG. 16B), YAP (FIG. 16C), DKC1 (FIG. 16D), or CDK4-CyclinD1 (FIG. 4B) were transfected into bovine 4C cells along with an integrase according to the methods described herein and subject to two rounds of puromycin selection. “(−) INT” or “−INT” indicates no co-transfection of integrase (i.e., transient transduction). In particular, the constructs were transfected into the 4C bovine cells at PDL 23.

Population doubling levels (PDLs) for each cell line was assessed and compared to control 4C bovine cells. Selection with puromycin was applied were noted.

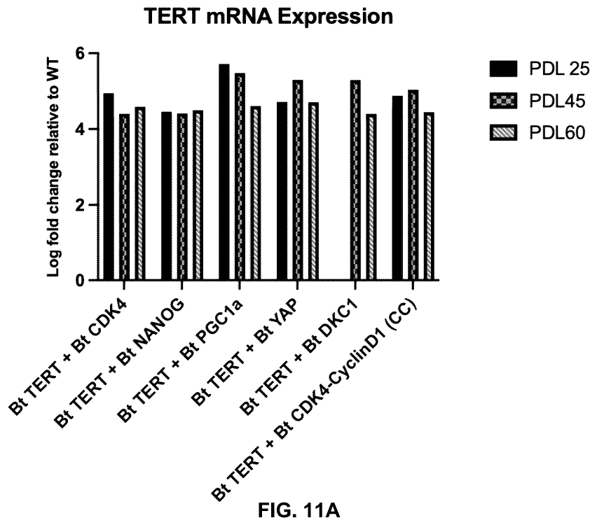

As shown in FIG. 10, wild type 4C bovine cells (i.e., cells that do not include a polynucleotide encoding TERT or a polynucleotide encoding a gene of interest (e.g., CDK4, NANOG, PGC1a, YAP, DKC1, or CDK4-CyclinD1) had a PDL of 50. As observed previously, 4C bovine cells containing TERT and a polynucleotide encoding a gene of interest (e.g., CDK4, NANOG, PGC1a, YAP, DKC1, or CDK4-CyclinD1) each had a PDL of greater than 75. This illustrates that TERT, in combination with other GOIs, causes 4C bovine cells to overcome cellular senescence.

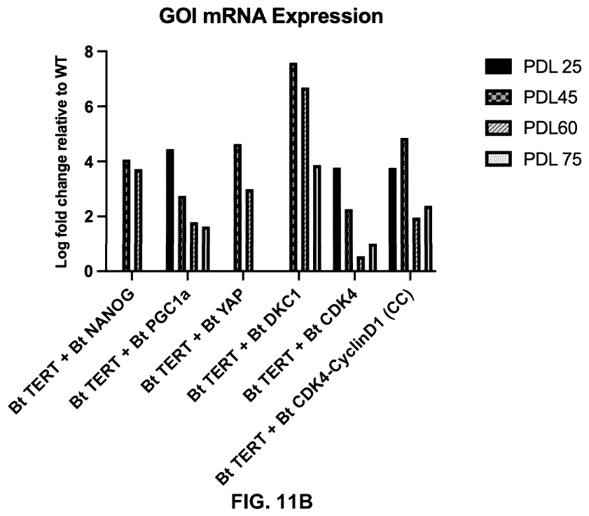

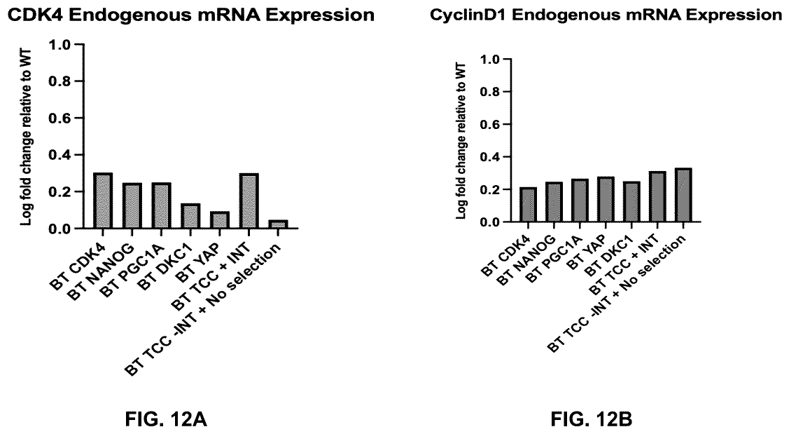

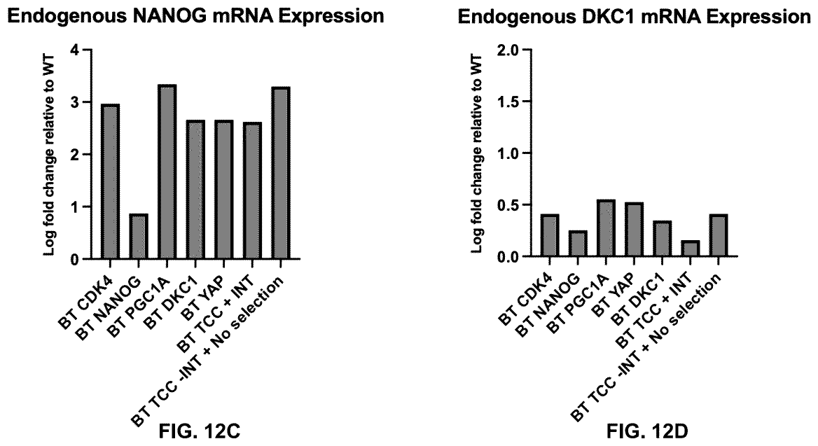

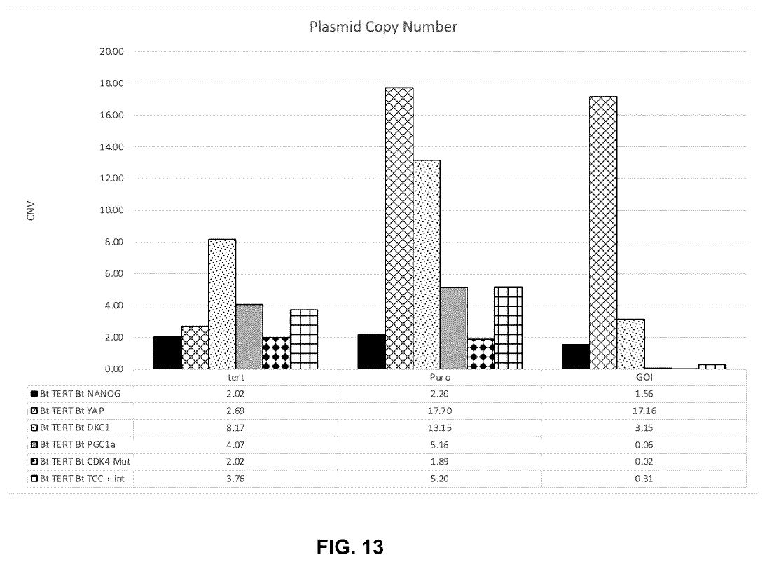

The bovine 4C lines comprising the various combinations of TERT and genes of interest were assessed for mRNA expression levels of TERT (FIG. 11A), mRNA expression of the genes of interest (FIG. 11B), mRNA expression of endogenous gene expression (FIGS. 12A-12D), as well as for copy number detection (FIG. 13).

FIG. 11A shows a histogram mRNA expression levels for TERT (log fold change relative to WT) in bovine 4C cells following each of the transduction conditions listed on the x-axis (Bt TERT+Bt CKD4; Bt TERT+Bt Nanog; Bt TERT+Bt PGC1a; Bt TERT+Bt YAP; Bt TERT+Bt DKC1; and Bt TERT+Bt CDK4-CyclinD1 (“CC”)). Cells were isolated and mRNA expression was assessed at population doubling level (PDL) 25, 45 and 60. Relative expression was measured using ΔΔCt as described previously. For each of the conditions measured (except Bt TERT+Bt DKC1; see FIG. 11A figure legend) TERT mRNA expression was maintained at least up until PDL 60.

FIG. 11B shows a histogram of mRNA expression levels for a gene of interest (GOI) (log fold change relative to WT) in bovine 4C cells following each of the transduction conditions listed on the x-axis (Bt TERT+Bt CKD4; Bt TERT+Bt Nanog; Bt TERT+Bt PGC1a; Bt TERT+Bt YAP; Bt TERT+Bt DKC1; and Bt TERT+Bt CDK4-CyclinD1 (“CC”)). Cells were isolated and mRNA expression was assessed at population doubling levels (PDLs) 25, 45, 60, and 75. Relative expression was measured using ΔΔCt as described previously. Gene of interest expression decreased as PDLs progressed.

FIGS. 12A-12D show histograms of mRNA expression levels for endogenous genes CDK4, CyclinD1, Nanog, and DKC1 (log fold change relative to WT) for bovine 4C cells following each of the transduction conditions listed on the x-axis (Bt TERT+Bt CKD4; Bt TERT+Bt Nanog; Bt TERT+Bt PGC1a; Bt TERT+Bt DKC1; Bt TERT+Bt YAP; Bt TERT+Bt CDK4-CyclinD1 (“CC”)+INT (Integrase); and Bt TERT+Bt CDK4-CyclinD1 (“CC”) (−) INT (Integrase)+No selection). Interestingly, dual TERT and gene of interest transfection in 4C cells increased expression of endogenous CDK4, CyclinD1, Nanog, and DKC1 compared to wild-type. As expected, INT boosted endogenous gene expression of CDK4, CyclinD, and NANOG specifically. Unexpectedly, the gene expression for Bt CDK4-CyclinD1 (“CC”)+INT (Integrase) compared to Bt TERT+Bt CDK4-CyclinD1 (“CC”) (−) INT (Integrase)+No selection) showed transient transfection without selection is sufficient to drive expression of endogenous genes CDK4, CyclinD1, Nanog, and DKC1 (see FIGS. 12A-12D). This suggests transient transfection strategy can be utilized for inducing endogenous expression of cell proliferation and cell cycle genes.

FIG. 13 shows a histogram of copy number (CNV) for bovine cells following the transduction conditions: Bt TERT+Bt CKD4; Bt TERT+Bt Nanog; Bt TERT+Bt PGC1a; Bt TERT+Bt DKC1; Bt TERT+Bt YAP; and Bt TERT+Bt CDK4-CyclinD1 (“CC”)+INT (Integrase).

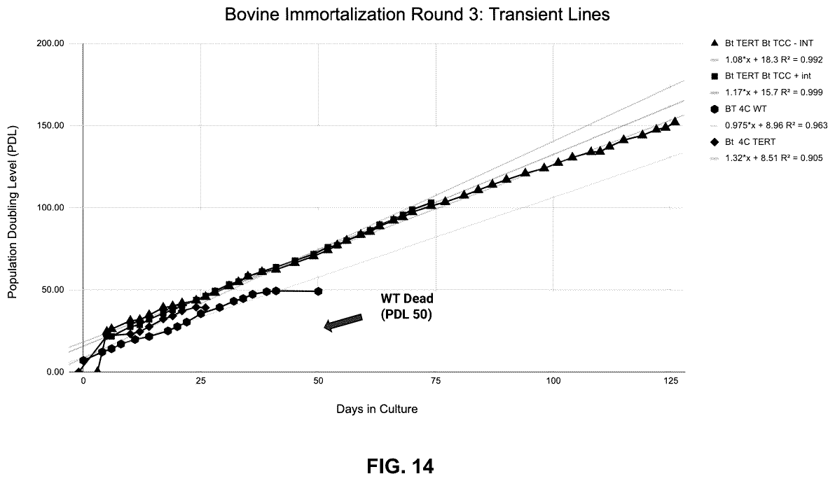

Due to the observed endogenous expression changes upon transient transfection, proliferation capacity was measured. PDLs were compared between 4C cell line transiently transfected with TERT/GOI without selection (see FIG. 14: Bt TERT Bt TCC−INT) and 4C dually transfected with TERT/GOI integrated into its genome (see FIG. 14: Bt TERT Bt TCC+int). Interestingly, PDLs were similar between 4C subjected to these conditions, showing that transient transfection not only increases endogenous GOI expression but also increases proliferation capacity and potential of 4C bovine cell lines. (see FIG. 14).

Once increased endogenous expression and proliferation was observed in transiently transfected 4C, plasmid copy number was evaluated to determine TERT/GOI integration. Surprisingly, as shown in FIG. 15, copy number in the bovine cell line transiently transfected with TERT/GOI not subjected to selection (i.e., Bt TERT Bt TCC−INT) at PDL 70, 85, 92, and 97 revealed that copy number for both TERT and the gene of interest decreased over time despite a constant rate of PDL (see slope for Bt TERT Bt TCC−INT in FIG. 14).

Overall, the following characteristics of dually transfected 4C were observed: (1) dual transfection of TERT/GOI curbs cellular senescence; (2) exogenous TERT expression is stabilized throughout progressing PDLs; (3) transient transfection without selection is sufficient to induce increased endogenous gene expression, proliferation capacity, and TERT expression.

In particular, transient dual TERT/GOI transfection strategy sufficiently induced similar characteristics when compared to TERT/GOI/integrase transfected 4C cells.

### 6. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

SEQUENCE APPENDIX

| SEQ | | |
|---|---|---|
| ID | | |
| NO: | Description | Sequence |
| 1 | Bos taurus | MPRAPRCRAVRALLRASYRQVLPLAAFVRR |
| | TERT | LRPQGHRLVRRGDPAAFRALVAQCLVCVPW |
| | Amino acid | DAQPPPAAPSFRQVSCLKELVARVVQRLCE |
| | | RGARNVLAFGFTLLAGARGGPPVAFTTSVR |
| | | SYLPNTVTDTLRGSGAWGLLLHRVGDDVLT |
| | | HLLSRCALYLLVPPTCAYQVCGPPLYDLRA |
| | | AAAAARRPTRQVGGTRAGFGLPRPASSNGG |
| | | HGEAEGLLEARAQGARRRRSSARGRLPPAK |
| | | RPRRGLEPGRDLEGQVARSPPRVVTPTRDA |
| | | AEAKSRKGDVPGPCRLFPGGERGVGSASWR |
| | | LSPSEGEPGAGACAETKRFLYCSGGGEQLR |
| | | RSFLLCSLPPSLAGARTLVETIFLDSKPGP |
| | | PGAPRRPRRLPARYWQMRPLFRKLLGNHAR |
| | | SPYGALLRAHCPLPASAPRAGPDHQKCPGV |
| | | GGCPSERPAAAPEGEANSGRLVQLLRQHSS |
| | | PWQVYGLLRACLRRLVPAGLWGSRHNERRF |
| | | LRNVKKLLSLGKHGRLSQQELTWKMKVQDC |
| | | AWLRASPGARCVPAAEHRQREAVLGRFLHW |
| | | LMGAYVVELLRSFFYVTETTFQKNRLFFFR |
| | | KRIWSQLQRLGVRQHLDRVRLRELSEAEVR |
| | | QHQEARPALLTSRLRFVPKPGGLRPIVNVG |
| | | CVEGAPAPPRDKKVQHLSSRVKTLFAVLNY |
| | | ERARRPGLLGASVLGMDDIHRA |
| 2 | Bos taurus | ATGCCGCGCGCGCCCAGGTGCCGGGCCGTG |
| | TERT | CGCGCCCTTCTGCGGGCCAGCTACCGGCAG |
| | Nucleic acid | GTGCTGCCCCTGGCCGCCTTCGTACGGCGC |
| | | CTGCGGCCCCAGGGCCACCGGCTTGTGCGG |
| | | CGCGGGGACCCGGCGGCCTTCCGCGCGCTG |
| | | GTGGCTCAGTGCTTGGTGTGCGTGCCCTGG |
| | | GACGCGCAGCCGCCCCCTGCCGCCCCGTCC |
| | | TTCCGCCAGGTGTCCTGCCTGAAGGAGCTG |
| | | GTGGCCAGAGTCGTGCAGAGGCTCTGCGAG |
| | | CGCGGCGCGAGGAACGTGCTGGCCTTCGGC |
| | | TTCACGCTGCTGGCCGGGGCCCGCGGCGGG |
| | | CCGCCCGTGGCCTTCACGACCAGCGTACGC |
| | | AGCTACCTGCCCAACACGGTAACCGACACG |
| | | CTGCGCGGCAGCGGCGCCTGGGGGCTGCTG |
| | | CTGCACCGCGTGGGCGACGACGTGCTCACC |
| | | CACCTGCTGTCGCGCTGCGCGCTCTACCTG |
| | | CTGGTGCCCCCGACCTGCGCCTACCAGGTG |
| | | TGTGGGCCGCCGCTCTATGACCTCCGCGCC |
| | | GCCGCCGCCGCCGCTCGTCGGCCCACGCGG |
| | | CAAGTGGGCGGGACCCGGGCGGGCTTCGGA |
| | | CTCCCGCGCCCGGCCTCGTCGAACGGCGGC |
| | | CACGGGGAGGCCGAAGGACTCCTGGAGGCG |
| | | CGGGCCCAGGGCGCGAGGCGGCGTCGCAGT |
| | | AGCGCGCGGGGACGACTGCCTCCAGCCAAG |
| | | AGGCCCAGGCGCGGCCTGGAGCCCGGGCGG |
| | | GATCTCGAAGGGCAGGTGGCCCGCAGCCCG |
| | | CCCCGCGTGGTGACACCTACCCGAGACGCT |
| | | GCGGAAGCCAAGTCTCGGAAGGGCGACGTG |
| | | CCCGGGCCCTGCCGCCTCTTCCCGGGCGGC |
| | | GAGCGGGGTGTCGGCTCCGCGTCCTGGCGG |
| | | CTGTCACCCTCGGAGGGCGAGCCGGGTGCC |
| | | GGAGCTTGCGCTGAGACCAAGAGGTTCCTT |
| | | TACTGCTCCGGCGGTGGCGAACAGCTGCGC |
| | | CGCTCCTTCCTGCTCTGCTCCCTGCCTCCC |
| | | AGCCTGGCCGGGGCGCGGACACTCGTGGAA |
| | | ACCATCTTTCTGGACTCGAAGCCCGGGCCG |
| | | CCAGGGGCTCCCCGCCGGCCGCGCCGCCTG |
| | | CCCGCGCGCTACTGGCAGATGCGGCCCCTG |
| | | TTCCGGAAACTGCTTGGGAACCACGCGCGG |
| | | AGCCCCTATGGCGCGCTGCTCAGGGCGCAC |
| | | TGCCCGCTGCCGGCCTCTGCGCCCCGGGGG |
| | | GGGCCAGACCATCAGAAGTGCCCTGGTGTT |
| | | GGGGGCTGCCCCTCTGAGAGGCCGGCCGCT |
| | | GCCCCCGAGGGCGAGGCGAACTCAGGGCGC |
| | | CTGGTCCAGCTGCTCCGCCAGCACAGCAGC |
| | | CCCTGGCAGGTGTACGGGCTCCTGCGGGCC |
| | | TGTCTTCGCCGCCTGGTGCCCGCCGGCCTC |
| | | TGGGGCTCCCGGCACAACGAGCGGCGCTTC |
| | | CTGCGGAACGTGAAGAAGCTCCTCTCCCTG |
| | | GGGAAGCACGGCAGGCTCTCGCAGCAGGAG |
| | | CTCACGTGGAAGATGAAGGTGCAGGACTGC |
| | | GCCTGGCTGCGCGCGAGCCCAGGGGCTCGC |
| | | TGCGTGCCCGCCGCGGAGCACCGCCAGCGC |
| | | GAGGCCGTCCTGGGTCGCTTCCTGCACTGG |
| | | CTGATGGGCGCCTACGTGGTGGAGCTGCTC |
| | | AGGAGCTTCTTCTACGTCACAGAGACCACG |
| | | TTCCAGAAGAACCGGCTCTTCTTCTTCCGG |
| | | AAGCGCATCTGGAGCCAGCTGCAGCGCCTG |
| | | GGCGTCAGACAACACTTAGACCGTGTGCGG |
| | | CTTCGAGAACTGTCAGAAGCAGAGGTCAGG |
| | | CAGCACCAGGAGGCCAGGCCGGCTCTGCTG |
| | | ACATCCAGGCTCCGTTTCGTCCCCAAGCCC |
| | | GGCGGGCTGCGGCCCATCGTGAACGTGGGC |
| | | TGTGTTGAGGGCGCCCCGGCACCGCCCAGA |
| | | GACAAGAAGGTGCAGCATCTCAGCTCACGG |
| | | GTCAAGACGCTGTTCGCGGTGCTGAACTAC |
| | | GAGCGAGCTCGGCGGCCTGGCCTCCTGGGG |
| | | GCCTCGGTGCTGGGCATGGACGACATCCAC |
| | | AGGGCCTG |
| 3 | Bos taurus | MHRTTRIKITELNPHLMCVLCGGYFIDATT |
| | BMI-1 | IIECLHSFCKTCIVRYLETSKYCPICDVQV |
| | Amino acid | HKTRPLLNIRSDKTLQDIVYKLVPGLFKNE |
| | | MKRRRDFYAAHPSADAANGSNEDRGEVADE |
| | | DKRIITDDEIISLSIEFFDQNRLDRKINKD |
| | | KEKSKEEVNDKRYLRCPAAMTVMHLRKFLR |
| | | SKMDIPNTFQIDVMYEEEPLKDYYTLMDIA |
| | | YIYTWRRNGPLPLKYRVRPTCKRMKISHQR |
| | | DGLTNTGELESDSGSDKANSPAGGIPSTSS |
| | | CLPSPSTPVQSPHPQFPHISSTMNGTSSSP |
| | | SGNHQSSFANRPRKSSVNGSSATSSG |
| 4 | Bos taurus | ATGCACAGGACTACACGCATTAAAATCACT |
| | BMI-1 | GAGCTCAATCCCCACTTGATGTGCGTCCTC |
| | Nucleic acid | TGCGGAGGTTACTTCATCGACGCCACTACA |
| | | ATCATTGAGTGTCTTCATAGTTTTTGCAAG |
| | | ACTTGCATTGTGAGATACCTTGAAACGTCA |
| | | AAGTATTGTCCTATTTGTGATGTCCAGGTG |
| | | CACAAGACCCGACCTTTGCTTAACATCAGG |
| | | TCAGATAAAACCCTTCAAGACATAGTCTAT |
| | | AAGCTGGTGCCGGGCCTCTTCAAGAACGAA |
| | | ATGAAGAGGCGGCGGGACTTCTATGCAGCC |
| | | CACCCAAGTGCTGATGCTGCGAATGGGTCA |
| | | AATGAAGATAGGGGTGAAGTGGCTGATGAA |
| | | GACAAAAGAATCATCACGGACGATGAGATA |
| | | ATTAGTCTTTCCATTGAATTCTTCGATCAA |
| | | AATAGATTGGACCGCAAAATCAACAAGGAC |
| | | AAAGAAAAAAGTAAAGAGGAGGTCAACGAC |
| | | AAGCGGTATTTGCGCTGTCCGGCAGCGATG |
| | | ACAGTGATGCACCTCAGGAAGTTCCTTAGA |
| | | TCTAAGATGGACATACCGAACACCTTTCAA |
| | | ATTGATGTTATGTATGAAGAGGAACCCCTC |
| | | AAAGATTACTACACGCTTATGGATATTGCC |
| | | TACATATATACATGGCGAAGGAACGGTCCA |
| | | CTTCCACTGAAATATAGGGTGCGACCTACG |
| | | TGCAAGCGCATGAAGATAAGCCACCAGCGG |
| | | GATGGACTTACAAATACGGGTGAACTGGAA |
| | | AGCGATAGCGGGTCTGATAAAGCCAATAGC |
| | | CCAGCTGGCGGAATACCATCTACATCCAGT |
| | | TGTCTGCCCAGTCCCTCCACGCCAGTGCAA |
| | | AGTCCACATCCACAATTTCCTCATATAAGC |
| | | TCCACGATGAACGGAACCTCTAGCAGTCCC |
| | | TCTGGAAACCATCAGAGTTCTTTCGCTAAC |
| | | CGACCGCGGAAGTCTTCAGTTAATGGTAGC |
| | | TCTGCAACAAGCTCCGGCTGA |
| 5 | Bos taurus | MATSRYEPVAEIGVGAYGTVYKACDPHSGH |
| | CDK4 | FVALKSVRVPNGGGAGGGLPISTVREVALL |
| | Amino acid | RRLEAFEHPNVVRLMDVCATARTDRETKVT |
| | | LVFEHVDQDLRTYLDKAPPPGLPVETIKDL |
| | | MRQFLRGLDFLHANCIVHRDLKPENILVTS |
| | | GGTVKLADFGLARIYSYQMALTPVVVTLWY |
| | | RAPEVLLQSTYATPVDMWSVGCIFAEMFRR |
| | | KPLFCGNSEADQLGKIFDLIGLPPEDDWPR |
| | | DVSLPRGAFSPRGPRPVQSVVPELEESGAQ |
| | | LLLEMLTFNPHKRISAFRALQHSYLHKAEG |
| | | DAE |
| 6 | Bos taurus | ATGGCCACAAGCCGATACGAACCAGTGGCA |
| | CDK4 | GAGATAGGTGTCGGGGCTTACGGAACTGTT |
| | Nucleic acid | TATAAGGCTTGTGATCCGCATTCCGGACAT |
| | | TTTGTCGCTCTGAAATCCGTGCGCGTGCCA |
| | | AATGGGGGAGGAGCAGGTGGAGGACTCCCA |
| | | ATTTCTACTGTTAGAGAGGTTGCATTGCTG |
| | | AGACGCCTGGAGGCCTTTGAGCATCCCAAC |
| | | GTGGTTAGGCTTATGGATGTTTGTGCCACG |
| | | GCCCGGACCGATCGGGAGACGAAAGTCACA |
| | | CTCGTCTTTGAGCACGTGGACCAGGATTTG |
| | | AGGACCTATCTGGACAAAGCCCCACCACCC |
| | | GGGCTGCCAGTTGAGACTATCAAGGACCTT |
| | | ATGAGACAGTTTCTTCGAGGCCTCGATTTC |
| | | CTCCACGCTAACTGCATCGTCCATAGAGAC |
| | | CTGAAGCCCGAGAATATTTTGGTTACCTCT |
| | | GGCGGCACCGTTAAATTGGCGGATTTCGGG |
| | | TTGGCACGAATTTATTCATATCAGATGGCC |
| | | CTGACCCCAGTCGTCGTTACGCTTTGGTAC |
| | | CGCGCCCCTGAGGTTCTCCTCCAAAGTACT |
| | | TACGCTACTCCTGTTGACATGTGGTCAGTT |
| | | GGATGTATTTTTGCTGAAATGTTTCGGAGA |
| | | AAACCGCTGTTTTGCGGGAACTCTGAAGCC |
| | | GATCAGTTGGGAAAAATATTTGACTTGATC |
| | | GGACTCCCGCCGGAAGATGACTGGCCCCGG |
| | | GATGTTTCTCTTCCGCGCGGGGCATTCTCT |
| | | CCCAGAGGTCCACGGCCAGTTCAGAGCGTC |
| | | GTGCCTGAACTTGAGGAATCAGGGGCTCAG |
| | | CTTCTGCTCGAGATGCTTACTTTTAATCCA |
| | | CACAAACGGATCAGCGCGTTCCGCGCACTG |
| | | CAACACTCCTATCTTCATAAGGCAGAAGGG |
| | | GACGCGGAGTGA |
| 7 | Bos taurus | MAHQLLCCEMETIRRAYPDANLLNDRVLRA |
| | Cyclin D1 | MLKAEETCAPSVSYFKCVQKEILPSMRKIV |
| | Amino acid | ATWMLEVCEEQKCEEEVFPLAMNYLDRFLS |
| | | LEPVKKSRLQLLGATCMFVASKMKETIPLT |
| | | AEKLCIYTDNSIRPDELLHMELVLVNKLKW |
| | | NLAAMTPHDFIEHFLSKMPVAEENKQIIRK |
| | | HAQTFVALCATDVKFISNPPSMVAAGSVAA |
| | | AAQGLHLGSANGFLSYHRLTRFLSKVIRCD |
| | | PDCLRACQEQIEALLESSLRQAQQQNLDPK |
| | | AAEEEEEEEEVDLACTPTDVRDVNI |
| 8 | Bos taurus | ATGGCACATCAGCTGCTGTGTTGTGAAATG |
| | Cyclin D1 | GAGACCATTCGGCGCGCCTATCCCGATGCT |
| | Nucleic acid | AATCTTCTTAATGATCGAGTGCTGCGGGCC |
| | | ATGCTGAAGGCTGAAGAAACGTGCGCTCCG |
| | | TCAGTCTCTTATTTCAAATGCGTGCAAAAG |
| | | GAGATTCTCCCTAGTATGCGAAAAATTGTG |
| | | GCCACATGGATGCTGGAGGTGTGTGAAGAG |
| | | CAGAAGTGTGAAGAAGAGGTCTTCCCACTT |
| | | GCAATGAACTATCTTGATAGATTTCTTAGT |
| | | CTGGAGCCGGTGAAAAAGTCACGGCTCCAG |
| | | TTGCTCGGAGCTACTTGCATGTTTGTTGCT |
| | | TCCAAAATGAAAGAAACAATCCCACTCACA |
| | | GCAGAGAAGTTGTGCATCTACACTGATAAC |
| | | TCTATTCGACCTGACGAGCTGCTTCACATG |
| | | GAGCTGGTTCTCGTCAATAAGCTGAAATGG |
| | | AACCTCGCAGCGATGACACCCCATGACTTC |
| | | ATAGAGCATTTTCTTTCAAAAATGCCTGTC |
| | | GCCGAGGAGAATAAGCAGATTATACGCAAA |
| | | CACGCCCAAACATTTGTGGCGCTTTGTGCG |
| | | ACCGACGTCAAATTTATCTCTAACCCACCA |
| | | TCAATGGTTGCCGCTGGCAGCGTTGCTGCC |
| | | GCGGCTCAAGGACTGCATCTGGGCTCAGCA |
| | | AACGGATTTTTGTCCTATCATCGCCTGACA |
| | | AGATTCCTGTCTAAAGTCATCAGATGCGAT |
| | | CCCGACTGCTTGCGAGCATGTCAGGAACAA |
| | | ATAGAGGCCCTGTTGGAGTCAAGCCTTAGA |
| | | CAGGCGCAACAGCAGAATCTCGACCCAAAA |
| | | GCAGCTGAGGAGGAGGAAGAGGAAGAAGAA |
| | | GTTGACTTGGCTTGTACACCCACGGATGTC |
| | | AGGGACGTTAACATTTGA |
| 9 | Bos taurus | MDEGYFCAALVGEDQPLCPDLPELDLSELD |
| | PGC1alpha | VNDLDTDSFLGGLKWCSDQSEIISNQYNNE |
| | Amino acid | PSNIFEKIDEENEANLLAVLTETLDSLPVD |
| | | EDGLPSFDALTDGDVTTENEASPSSMPDGT |
| | | PPPQEAEEPSLLKKLLLAPANTQLSYNECS |
| | | GLSTQNHANHNHRIRTNPAVVKTENSWSNK |
| | | AKSICQQQKPQRRPCSELLKYLTTNDDPPH |
| | | TKPTENRNSSRDKCTSKKKAHTQSQTQHLQ |
| | | AKPTTLSLPLTPESPNDPKGSPFENKTIER |
| | | TLSVELSGTAGLTPPTTPPHKANQDNPFRA |
| | | SPKLKPSCKTVVPPPSKKARYSESSCTQGS |
| | | NSTKKGPEQSELYAQLSKTSVLTSGHEERK |
| | | AKRPSLRLFGDHDYCQSINSKTEILVSTSQ |
| | | ELHDSRQLENKDAPSSNGPGQIHSSTDSDP |
| | | CYLRETAEVSRQVSPGSTRKQLQDQEIRAE |
| | | LNKHFGHPSQAVFDDKADKTSELRDSDFSN |
| | | EQFSKLPMFINSGLAMDGLFDDSEDESDKL |
| | | NSPWDGTQSYSLFDVSPSCSSFNSPCRDSV |
| | | SPPKSLFSQRPQRMRSRSRSFSRHRSCSRS |
| | | PYSRSRSRSPGSRSSSRSCYYYESGHCRHR |
| | | THRNSPLCASRSRSPHSRRPRYDSYEEYQH |
| | | ERLKREEYRREYEKRESERAKQRERQRQKA |
| | | INQQVSSHFPLQEERRVIYVG |
| 10 | Bos taurus | ATGGATGAGGGGTACTTTTGTGCTGCACTC |
| | PGC1alpha | GTCGGCGAGGACCAACCCCTTTGCCCGGAC |
| | Nucleic acid | CTCCCCGAGTTGGACCTCAGTGAGCTTGAC |
| | | GTCAACGATTTGGACACGGATAGCTTTCTG |
| | | GGTGGGCTTAAATGGTGTTCTGACCAATCC |
| | | GAGATCATATCCAATCAGTATAATAACGAA |
| | | CCGAGTAACATTTTCGAGAAAATAGATGAG |
| | | GAGAATGAAGCAAACCTTCTGGCTGTGCTC |
| | | ACGGAGACCTTGGATTCCCTCCCTGTGGAC |
| | | GAGGACGGTCTTCCTAGCTTTGACGCGCTC |
| | | ACAGATGGAGATGTTACAACAGAGAATGAA |
| | | GCCTCCCCTTCAAGTATGCCAGATGGAACT |
| | | CCACCCCCGCAAGAAGCTGAGGAACCTTCC |
| | | CTGCTTAAGAAACTGCTGTTGGCTCCGGCA |
| | | AACACACAACTCTCATATAATGAATGCTCT |
| | | GGGCTGTCAACCCAAAACCATGCGAATCAT |
| | | AACCACAGAATTAGGACAAATCCAGCGGTC |
| | | GTGAAGACGGAAAATTCCTGGTCTAATAAG |
| | | GCAAAGTCTATATGTCAACAACAGAAGCCG |
| | | CAGAGAAGGCCCTGTTCTGAACTGCTTAAA |
| | | TATCTTACCACGAACGACGACCCCCCACAC |
| | | ACGAAGCCCACTGAAAACCGAAATTCTTCA |
| | | AGGGATAAATGTACCTCTAAAAAAAAGGCC |
| | | CATACACAATCCCAGACTCAACATTTGCAA |
| | | GCTAAACCGACTACGCTTTCTCTTCCCCTC |
| | | ACTCCGGAAAGCCCCAACGACCCCAAAGGT |
| | | TCCCCATTTGAGAATAAAACGATAGAACGC |
| | | ACCCTGTCCGTCGAGTTGTCAGGTACGGCC |
| | | GGCCTTACTCCGCCTACTACTCCACCTCAT |
| | | AAGGCGAACCAAGATAATCCCTTCAGAGCT |
| | | TCACCAAAGCTTAAGCCAAGTTGCAAAACT |
| | | GTCGTCCCCCCTCCGTCTAAGAAAGCCCGC |
| | | TATTCCGAAAGTTCATGCACCCAAGGCTCT |
| | | AACTCTACCAAAAAGGGGCCTGAACAGTCA |
| | | GAATTGTATGCACAGTTGAGTAAAACGAGC |
| | | GTTCTCACCTCTGGACATGAGGAACGAAAG |
| | | GCTAAGCGGCCCTCCCTTAGGTTGTTCGGG |
| | | GACCATGACTACTGCCAGTCCATAAATTCC |
| | | AAAACAGAAATACTTGTGTCCACTTCACAA |
| | | GAGCTTCATGACTCTAGACAACTTGAAAAC |
| | | AAGGATGCTCCTTCAAGCAATGGTCCCGGG |
| | | CAGATTCACAGTTCTACCGACAGCGACCCC |
| | | TGTTATCTTAGAGAGACAGCGGAGGTTAGT |
| | | AGGCAGGTGTCCCCCGGATCAACACGAAAA |
| | | CAGCTCCAAGACCAGGAAATACGAGCGGAG |
| | | CTGAATAAACACTTTGGCCACCCGAGCCAA |
| | | GCTGTCTTCGACGACAAAGCCGATAAAACT |
| | | AGCGAGCTGAGGGATAGCGACTTCTCTAAT |
| | | GAACAATTCTCTAAACTTCCAATGTTCATA |
| | | AATAGCGGCCTCGCTATGGACGGCCTCTTC |
| | | GATGATTCCGAGGACGAATCTGACAAGTTG |
| | | AATAGTCCCTGGGACGGGACCCAAAGTTAC |
| | | TCCCTGTTCGATGTGTCACCCTCATGTTCC |
| | | TCATTCAACTCCCCGTGTAGAGACTCCGTC |
| | | TCCCCTCCAAAAAGCCTTTTTAGTCAGCGG |
| | | CCTCAAAGGATGCGATCTCGCAGTCGATCC |
| | | TTTAGTAGGCACAGAAGTTGTTCAAGATCT |
| | | CCATATTCTCGGTCACGGTCTAGAAGTCCT |
| | | GGTTCCAGAAGTTCATCCAGATCATGTTAC |
| | | TACTACGAAAGCGGACATTGTAGGCACAGG |
| | | ACCCACCGGAATTCTCCACTGTGCGCTAGT |
| | | CGAAGTCGATCTCCTCATAGCAGACGGCCA |
| | | CGATATGACTCCTACGAAGAGTACCAGCAC |
| | | GAGCGGTTGAAGCGGGAAGAGTACCGGCGC |
| | | GAGTACGAAAAAAGGGAAAGCGAAAGAGCC |
| | | AAACAGCGCGAGAGGCAGAGACAAAAAGCG |
| | | ATTAATCAACAGGTCAGTAGCCATTTCCCT |
| | | CTGCAAGAGGAACGCAGGGTGATTTATGTC |
| | | GGA |
| 11 | Bos taurus | MSVGPACPQSLLGPEASNSRESSPMPEESY |
| | Nanog | VSLQTSSADTLDTDTVSPLPSSMDLLIQDS |
| | Amino acid | PDSSTSPRVKPLSPSVEESTEKEETVPVKK |
| | | QKIRTVFSQTQLCVLNDRFQRQKYLSLQQM |
| | | QELSNILNLSYKQVKTWFQNQRMKCKKWQK |
| | | NNWPRNSNGMPQGPAMAEYPGFYSYHQGCL |
| | | VNSPGNLPMWGNQTWNNPTWSNQSWNSQSW |
| | | SNHSWNSQAWCPQAWNNQPWNNQFNNYMEE |
| | | FLQPGIQLQQNSPVCDLEATLGTAGENYNV |
| | | IQQTVKYFNSQQQITDLFPNYPLNIQPEDL |
| 12 | Bos taurus | ATGAGCGTGGGGCCGGCTTGCCCTCAATCT |
| | Nanog | CTGCTTGGCCCGGAGGCCTCTAATAGCCGT |
| | Nucleic acid | GAGAGCAGTCCTATGCCTGAGGAAAGTTAT |
| | | GTATCCCTGCAAACCTCCAGTGCCGATACG |
| | | TTGGACACCGATACCGTGTCACCCCTGCCA |
| | | TCTTCAATGGACCTTCTGATTCAAGACTCA |
| | | CCCGATAGCTCCACCAGTCCCCGCGTGAAG |
| | | CCCCTGTCCCCAAGCGTTGAAGAGAGCACC |
| | | GAGAAGGAAGAGACAGTCCCTGTGAAAAAG |
| | | CAGAAGATCCGGACTGTTTTCAGCCAGACG |
| | | CAATTGTGCGTGTTGAACGACCGCTTCCAG |
| | | CGTCAGAAGTACCTGAGCCTGCAACAGATG |
| | | CAGGAGCTGAGTAACATCCTCAACCTGTCC |
| | | TACAAACAGGTCAAGACCTGGTTCCAGAAT |
| | | CAACGGATGAAGTGTAAGAAATGGCAAAAG |
| | | AATAACTGGCCCCGTAACTCCAATGGGATG |
| | | CCCCAAGGTCCCGCTATGGCAGAATACCCA |
| | | GGGTTCTACTCCTACCACCAGGGCTGCCTG |
| | | GTAAACAGCCCTGGCAACCTCCCTATGTGG |
| | | GGCAACCAGACTTGGAATAACCCTACATGG |
| | | TCTAATCAGAGTTGGAACTCTCAGAGCTGG |
| | | TCTAATCACTCTTGGAACTCACAAGCCTGG |
| | | TGCCCTCAAGCTTGGAATAACCAGCCATGG |
| | | AATAACCAGTTCAATAACTACATGGAAGAG |
| | | TTCCTGCAGCCAGGTATTCAGCTGCAACAG |
| | | AACAGCCCCGTGTGTGACCTGGAGGCCACC |
| | | CTGGGAACCGCCGGCGAGAACTATAATGTG |
| | | ATCCAACAGACCGTCAAGTATTTCAATTCT |
| | | CAGCAACAGATCACGGACCTGTTCCCGAAC |
| | | TACCCTCTGAACATCCAGCCTGAGGACTTG |
| | | TAA |
| 13 | Bos taurus | MADAEVLILPKKHKKKKERKSLPEEAVAEI |
| | DKC1 | QHAEEFLIKPESRVAQLDTSQWPLLLKNFD |
| | Amino acid | KLNVRTTHYTPLPCGSNPLKREIGDYIRTG |
| | | FINLDKPSNPSSHEVVAWIRRILRVEKTGH |
| | | SGTLDPKVTGCLIVCIERATRLVKSQQSAG |
| | | KEYVGIVRLHNAIEGGTQLSRALETLTGAL |
| | | FQRPPLIAAVKRQLRVRTIYESKMIEYDPE |
| | | RRLGIFWVSCEAGTYIRTLCVHLGLLLGVG |
| | | GQMQELRRVRSGVMSEKDHMVTMHDVLDAQ |
| | | WLYDNHKDESYLRRVVYPLEKLLTSHKRLV |
| | | MKDSAVNAICYGAKIMLPGVLRYEDGIEVN |
| | | QEIVVITTKGEAICMAIALMTTAVISTCDH |
| | | GIVAKIKRVIMERDTYPRKWGLGPKASQKK |
| | | LMIKQGLLDKHGKPTDSTPATWMQEYVDYS |
| | | NSAKKDVPPKAVKATPVVAEVVKTPKRKRE |
| | | SESEDTSPAAPQVVKKEKKKKKKEKAKAAA |
| | | EKPGAGDSDSTKKKKKKKIKAEMVSE |
| 14 | Bos taurus | ATGGCAGATGCTGAAGTTCTTATTCTCCCT |
| | DKC1 | AAGAAACACAAAAAGAAAAAAGAACGCAAA |
| | Nucleic acid | TCACTCCCAGAGGAGGCCGTTGCAGAGATA |
| | | CAACATGCGGAAGAGTTCCTGATCAAACCA |
| | | GAATCTAGAGTTGCTCAACTGGATACATCT |
| | | CAATGGCCTTTGCTGCTGAAAAATTTCGAT |
| | | AAACTCAACGTCAGAACAACCCACTATACC |
| | | CCGCTCCCGTGTGGGTCTAATCCGCTTAAA |
| | | CGAGAAATCGGTGACTACATTCGGACAGGT |
| | | TTTATTAATCTCGATAAGCCTAGCAACCCC |
| | | TCTAGTCACGAGGTTGTCGCTTGGATCCGA |
| | | AGGATATTGAGGGTCGAGAAGACAGGGCAC |
| | | TCAGGTACCCTTGATCCCAAAGTCACCGGC |
| | | TGCCTCATAGTTTGCATCGAAAGAGCCACT |
| | | CGACTGGTTAAATCCCAACAATCTGCAGGA |
| | | AAAGAGTATGTTGGCATAGTCAGGCTTCAT |
| | | AATGCGATTGAGGGTGGCACCCAACTCTCC |
| | | AGGGCACTCGAAACACTTACGGGGGCACTT |
| | | TTCCAGCGACCACCTCTTATAGCGGCAGTC |
| | | AAACGGCAACTCCGAGTTAGGACGATATAC |
| | | GAGAGCAAGATGATCGAGTATGATCCTGAG |
| | | AGACGGCTTGGTATCTTTTGGGTTAGTTGT |
| | | GAGGCCGGCACGTATATCCGAACGCTTTGT |
| | | GTGCACTTGGGGTTGCTGCTTGGGGTCGGG |
| | | GGCCAAATGCAGGAATTGCGGCGGGTGCGG |
| | | TCAGGAGTGATGTCAGAGAAGGACCATATG |
| | | GTGACGATGCATGACGTCCTCGATGCCCAG |
| | | TGGCTTTACGACAATCACAAAGATGAGTCA |
| | | TACCTTAGGCGAGTTGTCTACCCACTCGAA |
| | | AAGTTGCTCACTTCCCACAAACGACTTGTG |
| | | ATGAAAGATAGCGCCGTGAACGCGATCTGC |
| | | TATGGCGCTAAGATAATGCTCCCCGGTGTC |
| | | CTCAGATACGAAGACGGGATTGAAGTTAAT |
| | | CAAGAAATCGTTGTCATTACGACTAAAGGA |
| | | GAGGCCATTTGCATGGCGATCGCCCTGATG |
| | | ACGACTGCCGTCATAAGTACATGCGATCAT |
| | | GGAATAGTGGCAAAAATCAAGAGGGTCATC |
| | | ATGGAAAGGGACACATACCCCCGGAAATGG |
| | | GGTCTGGGACCGAAAGCCTCTCAAAAGAAA |
| | | CTGATGATTAAACAGGGACTTCTTGACAAA |
| | | CATGGTAAACCGACTGATAGCACTCCCGCT |
| | | ACATGGATGCAAGAATATGTGGACTATTCT |
| | | AATTCCGCTAAAAAGGATGTGCCGCCTAAA |
| | | GCAGTGAAAGCGACCCCCGTCGTTGCTGAA |
| | | GTGGTTAAAACGCCAAAACGCAAAAGAGAG |
| | | TCTGAATCAGAAGACACTTCTCCCGCAGCG |
| | | CCGCAGGTCGTCAAAAAGGAGAAGAAAAAA |
| | | AAGAAGAAAGAGAAGGCGAAAGCTGCCGCT |
| | | GAGAAGCCGGGTGCCGGTGACAGCGATAGT |
| | | ACAAAGAAGAAAAAAAAAAAGAAAATCAAG |
| | | GCTGAAATGGTTTCTGAGTAG |
| 15 | Bos taurus | MDPGPPPPQPAPQGQGPPPAQAPQGQGPPS |
| | YAP | APGQPAPPGPPAAPQAPPAGHQIVHVRGDS |
| | Amino acid | ETDLEALFNAVMNPKTANVPQTVPMRLRKL |
| | | PDSFFKPPEPKSHSRQASTDAGTAGALTPQ |
| | | HVRAHSSPASLQLGAVSPGTLTPTGVVSGP |
| | | AAAPAAQHLRQSSFEIPDDVPLPAGWEMAK |
| | | TSSGQRYFLNHIDQTTTWQDPRKAMLSQMN |
| | | VTAPTSPPVQQNMMNSASGPLPDGWEQAMT |
| | | QDGEIYYINHKNKTTSWLDPRLDPRFAMNQ |
| | | RISQSAPVKQPPPLAPQSPQGGVLGGGSSN |
| | | QQQQMRLQQLQMEKERLRLKQQELLRQVRP |
| | | QAMRNINPSTANSPKCQELALRSQLPTLEQ |
| | | DGGTQNPVSSPGMSQELRTMTTNSSDPFLN |
| | | SGTYHSRDESTDSGLSMSSYSVPRTPDDFL |
| | | NSVDEMDTGDTINQSTLPSQQNRFPDYLEA |
| | | IPGTNVDLGTLEGDGMNIEGEELMPSLQEA |
| | | LSSDILNDMESVLAATKLDKESFLTWL |
| 16 | Bos taurus | ATGGATCCTGGACCGCCTCCCCCGCAGCCT |
| | YAP | GCGCCCCAGGGACAGGGGCCACCCCCAGCG |
| | Nucleic acid | CAGGCCCCACAAGGCCAGGGTCCCCCAAGC |
| | | GCTCCAGGCCAGCCTGCACCACCCGGCCCT |
| | | CCCGCTGCACCCCAGGCCCCACCTGCCGGT |
| | | CACCAGATCGTCCACGTCCGCGGGGACTCC |
| | | GAGACAGATCTGGAAGCTCTCTTTAACGCG |
| | | GTGATGAACCCAAAGACGGCAAATGTGCCT |
| | | CAGACCGTCCCTATGCGCCTGAGGAAGCTG |
| | | CCCGACTCCTTTTTCAAACCTCCGGAGCCA |
| | | AAATCCCATTCTCGCCAGGCCTCCACGGAT |
| | | GCCGGCACCGCGGGCGCACTGACGCCCCAG |
| | | CACGTCAGGGCTCATTCTAGCCCTGCCAGC |
| | | CTTCAGCTCGGCGCTGTGAGCCCCGGGACC |
| | | CTGACCCCTACGGGAGTTGTGTCCGGCCCC |
| | | GCCGCAGCCCCTGCCGCACAGCACCTGCGC |
| | | CAATCCTCTTTTGAGATTCCCGATGACGTG |
| | | CCTCTGCCCGCCGGTTGGGAGATGGCTAAG |
| | | ACCTCTAGCGGCCAGAGGTACTTCCTCAAT |
| | | CACATTGACCAGACTACCACTTGGCAGGAC |
| | | CCCCGGAAGGCGATGCTTTCTCAGATGAAC |
| | | GTGACAGCGCCGACTTCCCCTCCCGTCCAA |
| | | CAGAACATGATGAACTCCGCCAGCGGCCCA |
| | | CTCCCCGATGGGTGGGAGCAGGCTATGACC |
| | | CAGGACGGTGAGATTTATTACATCAACCAC |
| | | AAGAACAAGACGACCTCTTGGCTGGATCCA |
| | | AGATTGGATCCGCGCTTCGCCATGAACCAG |
| | | AGAATCTCACAGTCCGCCCCTGTGAAGCAG |
| | | CCGCCCCCGTTGGCGCCGCAAAGCCCTCAG |
| | | GGCGGAGTGCTGGGCGGTGGATCTTCCAAC |
| | | CAGCAACAGCAAATGCGCTTGCAGCAATTG |
| | | CAGATGGAAAAAGAGAGGCTGCGCCTCAAA |
| | | CAACAGGAACTTCTGAGGCAGGTGCGCCCC |
| | | CAGGCCATGAGGAACATCAACCCGTCCACC |
| | | GCTAATTCTCCTAAGTGCCAGGAACTGGCT |
| | | CTGCGCTCTCAGCTGCCCACGCTGGAGCAG |
| | | GACGGCGGGACCCAGAATCCTGTTAGTTCT |
| | | CCGGGAATGAGCCAAGAACTGCGTACTATG |
| | | ACTACAAACAGCTCCGACCCATTCTTGAAC |
| | | AGCGGCACCTACCACTCTCGTGATGAGAGC |
| | | ACCGACTCAGGATTGTCCATGAGCTCCTAT |
| | | AGCGTGCCTCGTACGCCCGATGACTTCCTC |
| | | AACAGCGTGGACGAAATGGATACCGGTGAC |
| | | ACCATCAACCAAAGCACCCTCCCAAGCCAG |
| | | CAAAACCGTTTCCCAGACTACTTGGAGGCA |
| | | ATCCCGGGGACAAACGTGGATCTTGGAACT |
| | | CTGGAGGGCGACGGGATGAACATCGAGGGC |
| | | GAAGAGCTCATGCCTTCCCTCCAGGAGGCG |
| | | CTGAGCTCCGACATCCTGAACGATATGGAA |
| | | TCCGTGTTGGCTGCAACCAAGCTGGACAAG |
| | | GAGTCCTTCTTGACATGGTTGTGA |
| 17 | GAPHD | GATGCTGCAGTTCCTCATTAGCACGGTCCT |
| | promoter | CACGCAGCACCGCACGGCCTGCTGGGCCGG |
| | | GCCCGGCCGGGAGCCTCAGAGCGGGGCCGT |
| | | TGGGCGGCCGTGCACCCCGGTATCGCGGTG |
| | | CGGGGACGGATATGGGACCCAGCGCTCCCC |
| | | AGCAGCTCGGGGCTGCGCGGCCAACACGCG |
| | | CAAGGGCTGAGGACGGGGAGCGGGCAGGCT |
| | | GCAGCCTCGCATCGCGCTCTTTGTCCCGCC |
| | | CGCGGCCAGACGCCGTTCTCTGGGTACCCG |
| | | CAGGAGCGCAATCCGCACCCACTGTACCGC |
| | | ATCGCGTGATGCAGCCGCATCGCCCCGGCC |
| | | TCGTCCGAGGACGCCACGGCCCCACGTGGG |
| | | GCTTTGTCTGCACGCAGCTCCGCGCCCCCC |
| | | GGCCCCGGGGCACCCCTCGCCGCCCCCACC |
| | | CCGCACGGGCACCGTGGTACGGCAGCAACC |
| | | CCCTAAGCCCACCCTGAGCCCAACGGCCGC |
| | | CCTTTGTTGCGAGGGGGGAGGGGGAAGGAG |
| | | AGGAGAGGGGGGGCCGTTTCGCCCGCACCG |
| | | TGCGCGCACGTAGCCCCGAGCCCGCCCCCG |
| | | CGCTGCCATTCGGCGCTGGCCCGGCGGGGG |
| | | GGGCGCGGGGCGCTGCCTCCGTGTCTATAA |
| | | AGGGCGGCGGCGCGGCACTGCGCCACCTTC |
| | | TCACTGCGCGCTGGGGCCGTTGACGTGCAG |
| | | CAGGTAACGGGGGGGGGGACGTGGGGGCGG |
| | | AGAAGCGTACCCCCCACCCACCCGCCTGTC |
| | | TGCCTGCGCCCCCCTGTAGGGGTTGGGGCT |
| | | GTAGGACCGCGAGGAGTGCGGCGGGGGAAG |
| | | GGGGGAGGGGGTCTCCGCGCAGGACCGCGT |
| | | GGTGCCCGGGGCACAAAGGCGGGGGCGGGC |
| | | GGCGCGAGATGAAGGGGGTGGGGGTTGGGG |
| | | GAGAGAGGGAGAACTGCGGGGGAGAGAGCT |
| | | CGATGGGGATGAAGGAAGGGATGGCGGGTC |
| | | GGGGGGCGGTGCCCAGAGCTCCGCAGCGCG |
| | | CGCGGTGGGGGGACGTTACTGGGCCGGCGG |
| | | TCTGCGTGCGGCACCGCACGTCTCACTGCC |

What is claimed is:

1. A method for extending the replicative capacity of a bovine cell, which is not immortal, in a population of bovine cells in cell culture, said method comprising:
(a) introducing into the bovine cell a first polynucleotide encoding telomere reverse transcriptase (TERT) operably linked to one or more regulatory elements that promote expression of TERT in said bovine cell and a second polynucleotide encoding a protein that modulates cell proliferation operably linked to one or more regulatory elements that promote expression of said protein in said bovine cell; and
(b) culturing the population of bovine cells in a cultivation infrastructure, wherein the population of bovine cells has a population doubling level (PDL) of at least 60 and the first polynucleotide or the second polynucleotide is not integrated into the genome of said bovine cell.

2. The method of claim 1 in which the protein that modulates cell proliferation is a BMI-1 protein, a CDK4 protein, a Cyclin D1 protein, a PGC1alpha protein, a nanog protein, a DCK1 protein, or a YAP protein.

3. The method of claim 1 in which neither the first polypeptide nor the second polypeptide are integrated into the genome of said bovine cell

4. The method of claim 1 in which the PDL is at least 100.

5. The method of claim 1 in which the doubling time of said population of bovine cells is less than 20 hours.

6. The method of claim 1 in which the population of bovine cells is isolated from bovine tissue or bovine muscle.

7. The method of claim 1 in which the population of bovine cells is from a master cell bank or subculture of a bovine cell culture.

8. The method of claim 1 in which the introduction of said first polypeptide or said second polypeptide is by transfection.

9. The method of claim 1 in which TERT has an amino acid sequence that is at least 80% identical to SEQ ID NO: 1 or the polynucleotide encoding TERT has a nucleotide sequence that is at least 80% identical to SEQ ID NO: 2.

10. The method of claim 2 in which the BMI-1 protein has an amino acid sequence that is at least 80% identical to SEQ ID NO: 3 or the polynucleotide encoding the BMI-1 protein has a nucleotide sequence that is at least 80% identical to SEQ ID NO: 4, the CDK4 protein has an amino acid sequence that is at least 80% identical to SEQ ID NO: 5 or the polynucleotide encoding the CDK4 protein has a nucleotide sequence that is at least 80% identical to SEQ ID NO: 6, the Cyclin D1 protein has an amino acid sequence that is at least 80% identical to SEQ ID NO: 7 or the polynucleotide encoding the Cyclin D1 protein has a nucleotide sequence that is at least 80% identical to SEQ ID NO: 8, the PGC1alpha protein has an amino acid sequence that is at least 80% identical to SEQ ID NO: 9 or the polynucleotide encoding the PGC1alpha protein has a nucleotide sequence that is at least 80% identical to SEQ ID NO: 10, the nanog protein has an amino acid sequence that is at least 80% identical to SEQ ID NO: 11 or the polynucleotide encoding the nanog protein has a nucleotide sequence that is at least 80% identical to SEQ ID NO: 12, the DKC1 protein has an amino acid sequence that is at least 80% identical to SEQ ID NO: 13 or the polynucleotide encoding the DKC1 protein has a nucleotide sequence that is at least 80% identical to SEQ ID NO: 14, the YAP protein has an amino acid sequence that is at least 80% identical to SEQ ID NO: 15, or the polynucleotide encoding the YAP protein has a nucleotide sequence that is at least 80% identical to SEQ ID NO: 16.

11. The method of claim 10 in which the one or more regulatory elements includes a GAPDH promoter, a CAG promoter, or an inducible promoter.

12. The method of claim 1 in which the first and second polynucleotides are not detectable in the population of cells at 5 doublings after said introduction.

13. A method of making cell-based meat suitable for consumption, comprising
(a) introducing into a bovine cell within a population of bovine cells that are not immortal a first polynucleotide encoding telomere reverse transcriptase (TERT) operably linked to one or more regulatory elements that promote expression of TERT in said bovine cell and a second polynucleotide encoding a protein that modulates cell proliferation operably linked to one or more regulatory elements that promote expression of said protein in said bovine cell; and
(b) culturing the population of bovine cells in one or more cultivation infrastructures for at least 60 doublings,
(c) inducing myogenic specific differentiation in the population of bovine cells after step (b), wherein the differentiated cells form myocytes and multinucleated myotubes; and
(d) culturing the myocytes and myotubes to generate skeletal muscle fibers, thereby producing a cultured edible product.

14. The method of claim 13 in which the protein that modulates cell proliferation is a BMI-1 protein, a CDK4 protein, a Cyclin D1 protein, a nanog protein, a PGC1alpha protein, a DCK1 protein, or a YAP protein.

15. The method of claim 13 in which neither the first polypeptide or the second polypeptide are integrated into the genome of said bovine cell

16. The method of claim 13 in which said inducing myogenic specific differentiation occurs after at least 100 doublings.

17. The method of claim 13 in which the population of bovine cells is isolated from bovine tissue or bovine muscle.

18. The method of claim 13 in which the population of bovine cells is from a master cell bank or subculture of a bovine cell culture.

19. The method of claim 13 in which the introduction of said first polypeptide or said second polypeptide is by transfection.

20. The method of claim 13 in which TERT has an amino acid sequence that is at least 80% identical to SEQ ID NO: 1 or the polynucleotide encoding TERT has a nucleotide sequence that is at least 80% identical to SEQ ID NO: 2.

21. The method of claim 14 in which the BMI-1 protein has an amino acid sequence that is at least 80% identical to SEQ ID NO: 3 or the polynucleotide encoding the BMI-1 protein has a nucleotide sequence that is at least 80% identical to SEQ ID NO: 4, the CDK4 protein has an amino acid sequence that is at least 80% identical to SEQ ID NO: 5 or the polynucleotide encoding the CDK4 protein has a nucleotide sequence that is at least 80% identical to SEQ ID NO: 6, the Cyclin D1 protein has an amino acid sequence that is at least 80% identical to SEQ ID NO: 7 or the polynucleotide encoding the Cyclin D1 protein has a nucleotide sequence that is at least 80% identical to SEQ ID NO: 8, the PGC1alpha protein has an amino acid sequence that is at least 80% identical to SEQ ID NO: 9 or the polynucleotide encoding the PGC1alpha protein has a nucleotide sequence that is at least 80% identical to SEQ ID NO: 10, the nanog protein has an amino acid sequence that is at least 80% identical to SEQ ID NO: 11 or the polynucleotide encoding the nanog protein has a nucleotide sequence that is at least 80% identical to SEQ ID NO: 12, the DKC1 protein has an amino acid sequence that is at least 80% identical to SEQ ID NO: 13 or the polynucleotide encoding the DKC1 protein has a nucleotide sequence that is at least 80% identical to SEQ ID NO: 14, the YAP protein has an amino acid sequence that is at least 80% identical to SEQ ID NO: 15, or the polynucleotide encoding the YAP protein has a nucleotide sequence that is at least 80% identical to SEQ ID NO: 16.

22. A population of bovine cells suitable for consumption produced by the method of claim 1 or claim 13.
